(19) United States
(12) Patent Application Publication
(10) Pub. No.: US 2003/0044422 A1
(43) Pub. Date: Mar. 6, 2003

(54) Ehrlichia ruminantium polypeptides, antigens, polynucleotides, and methods of use

(76) Inventors: Anthony F. Barbet, Archer, FL (US); William M. Whitmire, Hamilton, MT (US); Sondra M. Kamper, Round Rock, TX (US); Bigboy H. Simbi, Harare (ZW); Roman Reddy Ganta, Manhattan, KS (US); Annie L. Moreland, Trenton, FL (US); Duncan M. Mwangi, Nairobi (KE); Travis C. McGuire, Pullman, WA (US); Suman M. Mahan, Harare (ZW)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/081,051

(22) Filed: Feb. 20, 2002

Related U.S. Application Data
(60) Provisional application No. 60/269,944, filed on Feb. 20, 2001.

(51) Int. Cl. A61K 48/00; C07H 21/04; A61K 39/02; C12N 1/21; C12N 15/74

(52) U.S. Cl. 424/190.1; 514/44; 536/23.7; 435/252.3; 435/69.3; 435/320.1

(57) ABSTRACT

Described are nucleic acid vaccines containing genes to protect animals or humans against rickettsial diseases. Also described are polypeptides and methods of using these polypetides to detect antibodies to pathogens.

This application claims priority to U.S. Provisional Application 60/269,944, filed Feb. 20, 2001, which is hereby incorporated by reference in its entirety, including all nucleic acid sequences, amino acid sequences, figures, tables, and drawings.

This invention was made with government support under USAID Grant No. LAG-G-00-93-00030-00. The government has certain rights in this invention.

### TECHNICAL FIELD

This invention relates to nucleic acid vaccines for rickettsial diseases of animals, including humans.

### BACKGROUND OF THE INVENTION

The Rickettsia are a group of small bacteria commonly transmitted by arthropod vectors to man and animals, in which they may cause serious disease. The pathogens causing human rickettsial diseases include the agent of epidemic typhus, Rickettsia prowazekii, which has resulted in the deaths of millions of people during wartime and natural disasters. The causative agents of spotted fever, e.g., Rickettsia rickettsii and Rickettsia conorii, are also included within this group. Recently, new types of human rickettsial disease caused by members of the tribe Ehrlichiae have been described.

Heartwater is another infectious disease caused by a rickettsial pathogen, namely Cowdria ruminantium (now identified taxonomically as Ehrlichia ruminantium, see Dumler et al, Int. J. Syst. Evol. Microbiol. [2001]51(Pt 6):2145-65), and is transmitted by ticks of the genus Amblyomma. The disease occurs throughout most of Africa and has an estimated endemic area of about 5 million square miles. In endemic areas, heartwater is a latent infection in indigenous breeds of cattle that have been subjected to centuries of natural selection. The problems occur where the disease contacts susceptible or naive cattle and other ruminants. Heartwater has been confirmed to be on the island of Guadeloupe in the Caribbean and is spreading through the Caribbean Islands. The tick vectors responsible for spreading this disease are already present on the American mainland and threaten the livestock industry in North and South America.

Once imported, the disease may become established because of the presence of Amblyomma ticks that can transmit the disease to ruminant reservoir hosts in wildlife. (Mahan, S. M.; T. F. Peter, B. H. Simbi, K. Kocan, E. Camus, A. F. Barbet, and M. J. Burridge [2000]J. Parasitol. 86:44-49.) Thus, there is a need for improved methods for diagnosis and control of cowdriosis. Traditionally, diagnosis relies on the recognition of clinical signs, which can be confused with other diseases, or microscopic examination of brain biopsies on postmortem. The latter is impractical for widespread use. Control of cowdriosis employs acaricides to control the tick vector, or vaccination of domestic ruminants by infection with live organisms followed by antibiotic treatment. If detected early, tetracycline or chloramphenicol treatment are effective against rickettsial infections, but symptoms are similar to numerous other infections and there are no satisfactory diagnostic tests (Helmick, C., K. Bernard, L. D'Angelo [1984]J. Infect. Dis. 150:480).

In acute cases of heartwater, animals exhibit a sudden rise in temperature, signs of anorexia, cessation of rumination, and nervous symptoms including staggering, muscle twitching, and convulsions. Death usually occurs during these convulsions. Peracute cases of the disease occur where the animal collapses and dies in convulsions having shown no preliminary symptoms. Mortality is high in susceptible animals. Angora sheep infected with the disease have a 90% mortality rate while susceptible cattle strains have up to a 60% mortality rate.

Animals which have recovered from heartwater are resistant to further homologous, and in some cases heterologous, strain challenge. It has similarly been found that persons recovering from a rickettsial infection may develop a solid and lasting immunity. Individuals recovered from natural infections are often immune to multiple isolates and even species. For example, guinea pigs immunized with a recombinant R. conorii protein were partially protected even against R. rickettsii (Vishwanath, S., G. McDonald, N. Watkins [1990]Infect. Immun. 58:646). It is known that there is structural variation in rickettsial antigens between different geographical isolates. Thus, a functional recombinant vaccine against multiple isolates would need to contain multiple epitopes, e.g., protective T and B cell epitopes, shared between isolates. It is believed that serum antibodies do not play a significant role in the mechanism of immunity against rickettsia (Uilenberg, G. [1983]Advances in Vet. Sci. and Comp. Med. 27:427-480; Du Plessis, Plessis, J. L. [1970]Onderstepoort J. Vet. Res. 37(3):147-150).

Vaccines based on inactivated or attenuated rickettsiae have been developed against certain rickettsial diseases, for example against R. prowazekii and R. rickettsii. However, these vaccines have major problems or disadvantages, including undesirable toxic reactions, difficulty in standardization, and expense (Woodward, T. [1981] “Rickettsial diseases: certain unsettled problems in their historical perspective,” In Rickettsia and Rickettsial Diseases, W. Burgdorfer and R. Anacker, eds., Academic Press, New York, pp. 17-40).

A vaccine currently used in the control of heartwater is composed of live infected sheep blood. This vaccine also has several disadvantages. First, expertise is required for the intravenous inoculation techniques required to administer this vaccine. Second, vaccinated animals may experience shock and so require daily monitoring for a period after vaccination. There is a possibility of death due to shock throughout this monitoring period, and the drugs needed to treat any shock induced by vaccination are costly. Third, blood-borne parasites may be present in the blood vaccine and be transmitted to the vaccinates. Finally, the blood vaccine requires a cold chain to preserve the vaccine.

Clearly, a safer, more effective vaccine that is easily administered would be particularly advantageous. For these reasons, and with the advent of new methods in biotechnology, investigators have concentrated recently on the development of new types of vaccines, including recombinant vaccines. However, recombinant vaccine antigens must be carefully selected and presented to the immune system such that shared epitopes are recognized. These factors have contributed to the search for effective vaccines.

A protective vaccine against rickettsiae that elicits a complete immune response can be advantageous. A few antigens which potentially can be useful as vaccines have now been identified and sequenced for various pathogenic rickettsia. The genes encoding the antigens and that can be employed to recombinantly produce those antigen have also been identified and sequenced. Certain protective antigens identified for R. rickettsii, R. conorii, and R. prowazekii (e.g., rOmpA and rOmpB) are large (>100 kDa), dependent on retention of native conformation for protective efficacy, but are often degraded when produced in recombinant systems. This presents technical and quality-control problems if purified recombinant proteins are to be included in a vaccine. The mode of presentation of a recombinant antigen to the immune system can also be an important factor in the immune response.

Nucleic acid vaccination has been shown to induce protective immune responses in non-viral systems and in diverse animal species (Special Conference Issue, WHO meeting on nucleic acid vaccines [1994]Vaccine 12:1491). Nucleic acid vaccination has induced cytotoxic lymphocyte (CTL), T-helper 1, and antibody responses, and has been shown to be protective against disease (Ulmer, J., J. Donelly, S. Parker et al. [ 1993]Science 259:1745). For example, direct intramuscular injection of mice with DNA encoding the influenza nucleoprotein caused the production of high titer antibodies, nucleoprotein-specific CTLs, and protection against viral challenge. Immunization of mice with plasmid DNA encoding the Plasmodium yoelii circumsporozoite protein induced high antibody titers against malaria sporozoites and CTLs, and protection against challenge infection (Sedegah, M., R. Hedstrom, P. Hobart, S. Hoffman [1994]Proc. Natl. Acad. Sci. USA 91:9866). Cattle immunized with plasmids encoding bovine herpesvirus 1 (BHV-1) glycoprotein IV developed neutralizing antibody and were partially protected (Cox, G., T. Zamb, L. Babiuk [1993]J. Virol. 67:5664).

### BRIEF SUMMARY OF THE INVENTION

The subject invention provides novel vaccines for conferring immunityto rickettsial infection, including Ehrlichia ruminantium. Also disclosed are novel nucleic acid compositions and methods of using those compositions, including those that confer immunity in a susceptible host. Also disclosed are novel materials and methods for diagnosing infections by Ehrlichia ruminantium in humans or animals.

One aspect of the subject invention concerns a nucleic acid, e.g., DNA or mRNA, vaccine containing the antigenic proteins disclosed herein. In one embodiment, the nucleic acid vaccines can be driven by the human cytomegalovirus (HCMV) enhancer-promoter. Accordingly, the subject invention concerns the discovery that DNA vaccines can induce protective immunity against rickettsial disease or death resulting therefrom.

The subject invention further concerns the use of the nucleic acids of the subject invention in diagnostic and therapeutic applications. The subject invention further concerns the proteins encoded by the exemplified genes, antibodies to these proteins, and the use of such antibodies and proteins in diagnostic and therapeutic applications.

In one embodiment of the subject invention, the polynucleotide vaccines are administered in conjunction with an antigen. In a preferred embodiment, the antigen is the polypeptide which is encoded by the polynucleotide administered as the polynucleotide vaccine. As a particularly preferred embodiment, the antigen is administered as a booster subsequent to the initial administration of the polynucleotide vaccine.

### BRIEF DESCRIPTION OF THE DRAWINGS

FIG. 1 depicts the reactivity of recombinant bacteria with immune sheep serum. The selected recombinants (1 through 27hw and 1 through 7gd) were reacted on nitrocellulose filters with E. ruminantium-immune serum from a sheep (1:4000 dilution) followed by detection with ¹²⁵I-Protein G and autoradiography. Colony numbering and colonies containing previously known genes (map2 and groES/groEL encoding heat shock proteins, HSP) are indicated. The reactions of six positive (containing map2) and six negative (containing pGEM-7zf+ vector with no insert DNA) control colonies on the same colony immunoblot are shown at the bottom.

FIGS. 2A-D show the polypeptides synthesized in vitro by transcription and translation from each recombinant plasmid DNA. Plasmid DNA from recombinant colonies 1-27hw, 1-7gd, or vector alone (V) was used to program peptide synthesis and incorporation of ³⁵S-methioninc. Two different exposures of the synthesis reactions are shown. Protein molecularweight standards (S) are on the right of each panel.

FIGS. 3A-C demonstrate the recognition of E. ruminantium 28 kDa and 37 kDa proteins by immune sheep serum. The antigen for Western blots was, in 3A, a 28 kDa recombinant protein derived by subcloning from 18hw (511-1,263); in 3B, E. ruminantium organisms; in 3C, a 37 kDa recombinant protein derived from 1hw (1,333-2,313). Antisera were 3A, (from left) positive and negative sheep immune serum to E. ruminantium; pre- and post-immunization serum from a rabbit immunized with the recombinant 28 kDa protein; pre- and post-immunization serum from a second rabbit immunized with the recombinant 28 kDa protein; 3B, negative and positive sheep immune serum to E. ruminantium; post-immunization serum from 2 rabbits immunized with the recombinant 28 kDa protein; 3C, positive and negative sheep immune serum to E. ruminantium; pre- and post-immunization serum from a rabbit immunized with the recombinant 37 kDa protein; pre- and post-immunization serum from a second rabbit immunized with the recombinant 37 kDa protein. The position of protein molecular weight standards is indicated on the side of the blots.

FIG. 4 shows the recognition of 26hw peptide repeat by infected sheep serum in ELISA. A 27-mer peptide containing 3 tandem 9-mer repeats was used to coat the wells of an ELISA plate and reacted with varying dilutions of sera from E. ruminantium immune (Pos.) or uninfected (Neg.) sheep.

FIGS. 5A-B depict the stimulation of PBMC from immune cattle by bacterial lysates from each of the 34 clones were tested in proliferation assays. PBMC were isolated from immune cattle (post) and also from animal #245 before infection with E. ruminantium (pre). Controls included stimulation of PBMC with concanavalin A (ConA), lysates of E. coli containing pGEM-7zf+ vector as negative control (Ec), or recombinant MAP1 protein as positive control (Mp1).

### BRIEF DESCRIPTION OF THE TABLES

Table 1 shows the colony groupings of recombinant colonies containing cross-hybridizing genes.

Table 2 provides annotated functions for genes identified in genomic cloning of E. ruminantium.

Table 3 depicts the survival rate of animals immunized with recombinant bacterial lysates.

### DETAILED DISCLOSURE OF THE INVENTION

In one embodiment, the subject invention concerns a novel strategy, termed nucleic acid vaccination, for eliciting an immune response against rickettsial disease. The subject invention also concerns novel compositions that can be employed according to this novel strategy for eliciting a protective immune response.

According to the subject invention, recombinant DNA or mRNA encoding an antigen of interest is inoculated directly into the human or animal host where an immune response is induced. Prokaryotic signal sequences may be deleted from the nucleic acid encoding an antigen of interest. Advantageously, problems of protein purification, as can be encountered with antigen delivery using live vectors, can be virtually eliminated by employing the compositions or methods according to the subject invention. Unlike live vector delivery, the subject invention can provide a further advantage in that the DNA or RNA does not replicate in the host, but remains episomal. See, for example, Wolff, J. A., J. J. Ludike, G. Acsadi, P. Williams, A. Jani [1992]Hum. Mol. Genet. 1:363. A complete immune response can be obtained as recombinant antigen is synthesized intracellularly and presented to the host immune system in the context of autologous class I and class II MHC molecules.

In one embodiment, the subject invention concerns nucleic acids and compositions comprising those nucleic acids that can be effective in protecting an animal from disease or death caused by rickettsia. For example, a nucleic acid vaccine of the subject invention has been shown to be protective against Ehrlichia ruminantium, the causative agent of heartwater in domestic ruminants. Accordingly, nucleotide sequences of rickettsial genes, as described herein, can be used as nucleic acid vaccines against human and animal rickettsial diseases.

In another embodiment of the subject invention, the polynucleotide vaccines are administered in conjunction with an antigen. In a preferred embodiment, the antigen is the polypeptide that is encoded by the polynucleotide administered as the polynucleotide vaccine. As a particularly preferred embodiment, the antigen is administered as a booster subsequent to the initial administration of the polynucleotide vaccine. In another embodiment of the invention, the polynucleotide vaccine is administered in the form of a “cocktail” that contains at least two of the nucleic acid vaccines of the subject invention. The “cocktail” may be administered in conjunction with an antigen or an antigen booster as described above.

The subject invention also provides nucleotide sequences encoding polypeptides of Ehrlichia ruminantium. The invention also relates to transcriptional gene products of these polynucleotide sequences, methods of detecting the presence of nucleic acids or polypeptides in samples suspected of containing E. ruminantium organisms, polypeptide and/or nucleic acid vaccines for the induction of an immune response to E. ruminantium in an individual, and kits for detecting, diagnosing, treating, or reducing the incidence of infection by E. ruminantium .

Thus, the subject of the present invention encompasses nucleotide sequences comprising:

apolynucleotide sequence selected from the group consisting of SEQ ID NOs:2,3,4, 6, 8, 10, 12, 13, 15, 17, 19, 20, 21, 23, 24, 25, 27, 29, 31, 32, 33, 34, 36, 38, 39, 41, 43, 45, 46, 47, 48, 49, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 91, 92, 93, 94, 95, 96, 98, 100, 101, 103, 104, 105, 106, 108, 110, 114, and 115 or the complements thereof;

b) a polynucleotide sequence having at least about 20% to 99.99% identity to a polynucleotide selected from the group consisting of SEQ ID Nos: a polynucleotide sequence selected from the group consisting of SEQ ID NOs:2, 3, 4, 6, 8, 10, 12, 13, 15, 17, 19, 20, 21, 23, 24, 25, 27, 29, 31, 32, 33, 34, 36, 38, 39, 41, 43, 45, 46, 47, 48, 49, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 91, 92, 93, 94, 95, 96, 98, 100, 101, 103, 104, 105, 106, 108, 110, 114, and 115 or the complements thereof,

c) a polynucleotide sequence encoding a polypeptide selected from the group consisting of SEQ ID NOs:5, 7, 9, 11, 14, 16, 18, 22, 26, 28, 30, 35, 37, 40, 42, 44, 50, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 7, 9, 11, 14, 16, 18, 22, 26, 28, 30, 35, 37, 40, 42, 44, 50, 55, 60, 62, 66, 71, 76, 80, 83, 86, 89, 97, 99, 102, 107, 109, 111, 112, 113, and 116; or

d) a polynucleotide sequence encoding a polypeptide encoded by the complement of SEQ ID NOs:3, 20, 24, 32, 33, 47, 51, 53, 56, 57, 63, 68, 72, 73, 77, 78, 90, 91, 93, 103, and 104; or

e) a polynucleotide sequence encoding a polypeptide fragment or variant of a), b), c) or d), wherein said fragment or variant has substantially the same serologic activity as the native polypeptide; or

f) a polynucleotide sequence encoding a polypeptide fragment or variant of a polypeptide encoded by the complement of a), b), c), d), or e), wherein said fragment or variant has substantially the same serologic activity as the native polypeptide.

Nucleotide sequence, polynucleotide or nucleic acid are understood to mean, according to the present invention, either a double-stranded DNA, a single-stranded DNA or products oftranscription of the said DNAs (e.g., RNA molecules). It should also be understood that the present invention does not relate to the genomic nucleotide sequences of E. ruminantium in their natural environment or natural state. The nucleic acid, polynucleotide, or nucleotide sequences of the invention have been isolated, purified (or partially purified), by separation methods including, but not limited to, ion-exchange chromatography, molecular size exclusion chromatography, affinity chromatography, or by genetic engineering methods such as amplification, cloning or subcloning.

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and, up to, including 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention.

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman {1988}Proc. Natl. Acad. Sci. USA 85(8):2444-2448; Altschul et al. [1990]J. Mol. Biol. 215(3):403-410; Thompson et al. [1994]Nucleic Acids Res. 22(2):4673-80; Higgins et al. [1996]Methods Enzymol. 266:383-402; Altschul et al. [1990]J. Mol. Biol. 215(3):403-410; Altschul et al. [1993]Nature Genetics 3:266-272).

The subject invention also provides nucleotide sequences complementary to the sequences disclosed herein. Thus, the invention is understood to include any DNA whose nucleotides are complementary to those of the sequence of the invention, and whose orientation is reversed (e.g., anti-sense sequences).

The present invention further comprises fragments of the sequences of the instant invention as well as fragments of the gene products contained within the polynucleotide sequences provided herein. Representative fragments of the polynucleotide sequences according to the invention will be understood to mean any nucleotide fragment having at least 8 successive nucleotides, preferably at least 12 successive nucleotides, and still more preferably at least 15 or at least 20 successive nucleotides of the sequence from which it is derived. The upper limit for such fragments is the total number of polynucleotides found in the full length sequence (or, in certain embodiments, of the full length open reading frame (ORF) identified herein). It is understood that such fragments refer only to portions of the disclosed polynucleotide sequences that are not listed in a publicly available database.

In some embodiments, the subject invention includes those fragments capable of hybridizing under stringent conditions with a nucleotide sequence according to the invention. Hybridization under conditions of high or intermediate stringency, are defined below. Thus, conditions are chosen such that they allow hybridization to be maintained between two complementary DNA fragments. Hybridization conditions described above for a polynucleotide of about 300 bases in size can be adapted by persons skilled in the art for larger- or smaller-sized oligonucleotides, according to the teaching of Sambrook et al. [1989].

Other embodiments provide for nucleic acid fragments corresponding to nucleotide sequences comprising full, or partial, open reading frames (ORF sequences). Also within the scope of the invention are those polynucleotide fragments encoding polypeptides reactive with antibodies found in the serum of individuals infected with E. ruminantium. Fragments according to the subject invention can be obtained, for example, by specific amplification (e.g., PCR amplification), digestion with restriction enzymes, of nucleotide sequences according to the invention. Such methodologies are well-known in the art and are taught, for example, by Sambrook et al. [1989]. Nucleic acid fragments according to the invention can also be obtained by chemical synthesis according to methods well known to persons skilled in the art.

Thus, the subject invention also provides nucleic acid based methods for the identification of the presence of an organism in a sample. These methods can utilize the nucleic acids of the subject invention and are well known to those skilled in the art (see, for example, Sambrook et al. [1989]. Among the techniques useful in such methods are enzymatic gene amplification (or PCR), Southern blots, Northern blots, or other techniques utilizing hybridization for the identification of polynucleotide sequences in a sample.

The subject invention also provides for modified nucleotide sequences. Modified nucleic acid sequences will be understood to mean any nucleotide sequence that has been modified, according to techniques well known to persons skilled in the art, and exhibiting modifications in relation to the native, naturally occurring nucleotide sequences. One non-limiting example of a “modified” nucleotide sequences includes mutations in regulatory and/or promoter sequences of a polynucleotide sequence that result in a modification of the level of expression of the polypeptide. A modified nucleotide sequence will also be understood to mean any nucleotide sequence encoding a modified polypeptide as defined below.

The subject invention also provides polypeptides encoded by nucleotide sequences according to the invention. In some embodiments, several polypeptides are encoded by a single polynucleotide provided herein. However, the invention is not limited to polypeptides encoded by ORFs in the sequences provided herein. The full scope of the instant invention includes polypeptides of strain variants, polymorphisms, allelic variants, and mutants.

Thus, the subject invention provides one or more isolated polypeptides comprising:

a) a polypeptide encoded by a polynucleotide sequence selected from the group consisting of SEQ ID NOs:2, 3, 4, 6, 8, 10, 12, 13, 15, 17, 19, 20, 21, 23, 24, 25, 27, 29, 31, 32, 33, 34, 36, 38, 39, 41, 43, 45, 46, 47, 48, 49, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 91, 92, 93, 94, 95, 96, 98, 100, 101, 103, 104, 105, 106, 108, 110, 114, and 115 or the complements thereof;

b) a polypeptide encoded by a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 6, 8, 10, 12, 13, 15, 17, 19, 20, 21, 23, 24, 25, 27, 29, 31, 32, 33, 34, 36, 38, 39, 41, 43, 45, 46, 47, 48, 49, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 91, 92, 93, 94, 95, 96, 98, 100, 101, 103, 104, 105, 106, 108, 110, 114, and 115 or the complements thereof;

c) a polypeptide selected from the group consisting of SEQ ID NOs:5, 7, 9, 11, 14, 16, 18, 22, 26, 28, 30, 35, 37, 40, 42, 44, 50, 55, 60, 62, 66, 71, 76, 80, 83, 86, 89, 97, 99, 102, 107, 109, 111, 112, 113, and 116 or the complement of a polynucleotide sequence encoding apolypeptide selected from the group consisting of SEQ ID NOs:5, 7, 9, 11, 14, 16, 18, 22, 26, 28, 30, 35, 37, 40, 42, 44, 50, 55, 60, 62, 66, 71, 76, 80, 83, 86, 89, 97, 99, 102, 107, 109, 111, 112, 113, and 116; or

d) a polypeptide encoded by a polynucleotide sequence encoding a polypeptide encoded by the complement of SEQ ID NOs:3, 20, 24, 32, 33, 47, 51, 53, 56, 57, 63, 68, 72, 73, 77, 78, 90, 91, 93, 103, and 104; or

e) a polypeptide encoded by a polynucleotide sequence encoding a polypeptide fragment or variant of a), b), c) or d), wherein said fragment or variant has substantially the same serologic activity as the native polypeptide; or

f) a polypeptide encoded by a polynucleotide sequence encoding a polypeptide fragment or variant of a polypeptide encoded by the complement of a), b), c), d), or e), wherein said fragment or variant has substantially the same serologic activity as the native polypeptide.

The subject invention also provides fragments of at least 5 amino acids of a polypeptide encoded by the polynucleotides of the instant invention. In some embodiments, the polypeptide fragments are reactive with antibodies found in the serum of an infected individual. In the context of the instant invention, the terms polypeptide, peptide and protein are used interchangeably; however, it should be understood that the invention does not relate to the polypeptides in natural form, that is to say that they are not taken in their natural environment but that they may have been isolated or obtained by purification from natural sources, obtained from host cells prepared by genetic manipulation (e.g., the polypeptides, or fragments thereof, are recombinantly produced by host cells, or by chemical synthesis). Polypeptides according to the instant invention may also contain non-natural amino acids, as will be described below.

A homologous polypeptide will be understood to designate a polypeptides exhibiting, in relation to the natural polypeptide, certain modifications. These modifications can include a deletion, addition, or substitution of at least one amino acid, a truncation, an extension, a chimeric fusion, a mutation, or polypeptides exhibiting post-translational modifications. Among the homologous polypeptides, those whose amino acid sequences exhibit between at least (or at least about) 20.00% to 99.99% (inclusive) identity to the native, naturally occurring polypeptide are another aspect of the invention. The aforementioned range of percent identity is to be taken as including, and providing written description and support for, any fractional percentage, in intervals of 0.01%, between 20.00% and, up to, including 99.99%. These percentages are purely statistical and differences between two polypeptide sequences can be distributed randomly and over the entire sequence length.

Homologous polypeptides can, alternatively, have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polypeptide sequences of the instant invention. The expression equivalent amino acid is intended here to designate any amino acid capable of being substituted for one of the amino acids in the basic structure without, however, essentially modifying the biological activities of the corresponding peptides and as will be defined later.

By way of example, amino acid substitutions can be carried out without resulting in a substantial modification of the biological activity of the corresponding modified polypeptides; for example, the replacement of leucine with valine or isoleucine, of aspartic acid with glutamic acid, of glutamine with asparagine, of arginine with lysine, and the like, the reverse substitutions can be performed without substantial modification of the biological activity of the polypeptides.

The subject invention also provides biologically active fragments of a polypeptide according to the invention and includes those peptides capable of eliciting an immune response directed against E. ruminantium, said immune response providing components (either antibodies or components of the cellular immune response (e.g., B-cells, helper, cytotoxic, and/or suppressor T-cells)) reactive with the biologically active fragment of a polypeptide, the intact, full length, unmodified polypeptide disclosed herein, or both the biologically active fragment of a polypeptide and the intact, full length, unmodified polypeptides disclosed herein.

Fragments, as described herein, can be obtained by cleaving the polypeptides of the invention with a proteolytic enzyme (such as trypsin, chymotrypsin, or collagenase) or with a chemical reagent, such as cyanogen bromide (CNBr). Alternatively, polypeptide fragments can be generated in a highly acidic environment, for example at pH 2.5. Such polypeptide fragments maybe equally well prepared by chemical synthesis or using hosts transformed with an expression vector according to the invention. The transformed host cells contain a nucleic acid, allowing the expression of these fragments, under the control of appropriate elements for regulation and/or expression of the polypeptide fragments.

Modified polypeptides according to the invention are understood to designate a polypeptide obtained by genetic recombination or by chemical synthesis as described below. Modified polypeptides contain at least one modification in relation to the normal polypeptide sequence. These modifications can include the addition, substitution, deletion of amino acids contained within the polypeptides of the instant invention.

Accordingly, in order to extend the life of the polypeptides according to the invention, it may be advantageous to use non-natural amino acids, for example in the D form, or alternatively amino acid analogs, for example sulfur-containing forms of amino acids. Alternative means for increasing the life of polypeptides can also be used in the practice of the instant invention. For example, polypeptides of the invention, and fragments thereof, can be recombinantly modified to include elements that increase the plasma, or serum half-life of the polypeptides of the invention. These elements include, and are not limited to, antibody constant regions (see for example, U.S. Pat. No. 5,565,335, hereby incorporated by reference in its entirety, including all references cited therein), or other elements such as those disclosed in U.S. Pat. Nos. 6,319,691; 6,277,375; or 5,643,570, each of which is incorporated by reference in its entirety, including all references cited within each respective patent. Alternatively, the polynucleotides and genes of the instant invention can be recombinantly fused to elements, well known to the skilled artisan, that are useful in the preparation of immunogenic constructs for the purposes of vaccine formulation.

The subject invention also provides detection probes (e.g., fragments of the disclosed polynucleotide sequences) for hybridization with a target sequence or the amplicon generated from the target sequence. Such a detection probe will advantageously have as sequence a sequence of at least 12, 15, 20, 25, 30, 35, 40, 45, 50, 55, 60, 65, 70, 75, 80, 85, 90, 95, or 100 nucleotides. The detection probes can also be used as labeled probe orprimerin the subject invention. Labeled probes or primers are labeled with a radioactive compound or with another type of label. Alternatively, non-labled nucleotide sequences maybe used directly as probes or primers; however, the sequences are generally labeled with a radioactive element (³²P, 35S, ³H, ¹²⁵I) or with a molecule such as biotin, acetylaminofluorene, digoxigenin, 5-bromo-deoxyuridine, or fluorescein to provide probes that can be used in numerous applications.

The nucleotide sequences according to the invention may also be used in analytical systems, such as DNA chips. DNA chips and their uses are well known in the art and (see for example, U.S. Pat. Nos. 5,561,071; 5,753,439; 6,214,545; Schena et al. [1996]BioEssays 18:427-31; Bianchi et al. [1997]Clin. Diagn. Virol. 8:199-208; each of which is hereby incorporated by reference in their entireties) and/or are provided by commercial vendors such as Affymetrix, Inc. (Santa Clara, Calif.).

Another aspect of the invention provides vectors for the cloning and/orthe expression of a polynucleotide sequence taught herein. Vectors of this invention can also comprise elements necessary to allow the expression and/or the secretion of the said nucleotide sequences in a given host cell. The vector can contain a promoter, signals for initiation and for termination of translation, as well as appropriate regions for regulation of transcription. In certain embodiments, the vectors can be stably maintained in the host cell and can, optionally, contain signal sequences directing the secretion of translated protein. These different elements are chosen according to the host cell used. Vectors can integrate into the host genome or, optionally, be autonomously-replicating vectors.

The subject invention also provides for the expression of a polypeptide, peptide, derivative, or analog encoded by a polynucleotide sequence disclosed herein. The disclosed sequences can also be regulated by a second nucleic acid sequence so that the protein or peptide is expressed in a host transformed with the recombinant DNA molecule. For example, expression of a protein or peptide may be controlled by any promoter/enhancer element known in the art. Promoters which may be used to control expression include, but are not limited to, the CMV promoter, the SV40 early promoter region (Bernoist and Chambon [1981] Nature 290:304-310), the promoter contained in the 3′ long terminal repeat of Rous sarcoma virus (Yamamoto et al. [1980] Cell 22:787-97), the herpes thymidine kinase promoter (Wagner et al. [1981] Proc. Natl Acad. Sci. U.S.A. 78:1441-1445), the regulatory sequences of the metallothionein gene (Brinster et al. [1982] Nature 296:39-42); prokaryotic vectors containing promoters such as the β-lactamase promoter (Villa-Kamaroff et al. [1978] Proc. Natl. Acad. Sci. U.S.A. 75:3727-3731), or the tac promoter (DeBoer et al. [1983] Proc. Natl. Acad. Sci. U.S.A. 80:21-25); see also, “Useful proteins from recombinant bacteria” [1980] Scientific American 242:74-94; plant expression vectors comprising the nopaline synthetase promoter region (Herrera-Estrella et al. [1983] Nature 303:209-213) or the cauliflower mosaic virus 35S RNA promoter (Gardner et al. [1981] Nucl. Acids Res. 9:2871), and the promoter of the photosynthetic enzyme ribulose biphosphate carboxylase (Herrera-Estrella et al. [1984] Nature 310:115-120); promoter elements from yeast or fungi such as the Gal 4 promoter, the ADC (alcohol dehydrogenase) promoter, PGK (phosphoglycerol kinase) promoter, and/or the alkaline phosphatase promoter

The vectors according to the invention are, for example, vectors of plasmid or viral origin. In a specific embodiment, a vector is used that comprises a promoter operably linked to a protein or peptide-encoding nucleic acid sequence contained within the disclosed polynucleotide sequences, one or more origins of replication, and, optionally, one or more selectable markers (e.g., an antibiotic resistance gene). Expression vectors comprise regulatory sequences that control gene expression, including gene expression in a desired host cell. Exemplary vectors for the expression of the polypeptides of the invention include the pET-type plasmid vectors (Promega) or pBAD plasmid vectors (Invitrogen) or those provided in the examples below. Furthermore, the vectors according to the invention are useful for transforming host cells so as to clone or express the nucleotide sequences of the invention.

The invention also encompasses the host cells transformed by a vector according to the invention. These cells may be obtained by introducing into host cells a nucleotide sequence inserted into a vector as defined above, and then culturing the said cells under conditions allowing the replication and/or the expression of the transfected nucleotide sequence.

The host cell may be chosen from eukaryotic or prokaryotic systems, such as for example bacterial cells, (Gram negative or Gram positive), yeast cells, animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691; 6,277,375; 5,643,570; or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

In other specific embodiments, the polypeptides, peptides or derivatives, or analogs thereof may be expressed as a fusion, or chimeric protein product (comprising the protein, fragment, analog, or derivative joined via a peptide bond to a heterologous protein sequence (e.g., a different protein)). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

Another embodiment of the subject invention provides for the use of polypeptides encoded by the polynucleotides of the subject invention for the induction of an immune response or protective immunity in a subject to which the polypeptides are administered. In this aspect of the invention, compositions containing polypeptide are administered to a subject in amounts sufficient to induce an immune response, and preferably, protective immunity. The polypeptides may be administered individually or in the form of a “cocktail” comprising at least two or more polypeptides according to the invention. The composition administered to the subject may, optionally, contain an adjuvant and may be delivered to the subject in any manner known in the art for the delivery of immunogen to a subject. Compositions may be formulated in any carriers, including for example, carriers described in E. W. Martin's Remington's Pharmaceutical Science, Mack Publishing Company, Easton, Pa.

The subject invention further concerns the proteins encoded by the exemplified genes, antibodies to these proteins, and the use of such antibodies and proteins in diagnostic and therapeutic applications.

Compositions comprising the subject polynucleotides can include appropriate nucleic acid vaccine vectors (plasmids), which are commercially available (e.g., Vical, San Diego, Calif.). In addition, the compositions can include a pharmaceutically acceptable carrier, e.g., saline. The pharmaceutically acceptable carriers are well known in the art and also are commercially available. For example, such acceptable carriers are described in E. W. Martin's Remington's Pharmaceutical Science, Mack Publishing Company, Easton, Pa.

The subject invention also concerns polypeptides encoded by the subject polynucleotides. These polypeptides can be used in a variety of well-known purposes, including (but not limited to) the induction of immune responses to the polypeptides, use in diagnostic assays or immunoassays, and/or use in the production of affinity matrices for the isolation and purification of specific antibodies.

The subject invention provides for diagnostic assays based upon Western blot formats or standard ELISA or RIA based detection formats. The subject invention also provides kits containing antibodies and polypeptides according to the invention. The antibodies or polypeptides can be coated onto a solid phase, such as an ELISA microtiter plate, dipstick, magnetic beads, and the like, and used as a sensitive reagent to accurately detect antibodies or polypeptides in individuals infected with E. ruminantium. By “kit”, is intended that the monoclonal antibody, or polypeptides, and any necessary reagents are contained in close confinement in the form of a ready-to-use test kit. The antibodies or polypeptides of the invention are useful in non-competitive ELISA, including double-sandwich ELISA assays, as well as competitive assays. However, other formats such as homogenous enzyme immunoassays may be developed.

Detection systems for the identification of infected individuals include antibody-based assays such as enzyme linked immunosorbent assays (ELISAs), radioimmunoassays (RIAs), lateral flow assays, immunochromatographic strip assays, automated flow assays, and assays utilizing antibody-containing biosensors. The assays and methods for conducting the assays are well-known in the art. The antibody-based assays can be considered to be of four types: direct binding assays, sandwich assays, competition assays, and displacement assays. In a direct binding assay, either the antibody or antigen is labeled, and there is a means of measuring the number of complexes formed. In a sandwich assay, the formation of a complex of at least three components (e.g., antibody-antigen-antibody) is measured. In a competition assay, labeled antigen and unlabelled antigen compete for binding to the antibody, and either the bound or the free component is measured. In a displacement assay, the labeled antigen is prebound to the antibody, and a change in signal is measured as the unlabelled antigen displaces the bound, labeled antigen from the receptor.

Lateral flow assays can be conducted according to the teachings of U.S. Pat. No. 5,712,170 and the references cited therein. U.S. Pat. No. 5,712,170, and the references cited therein, are hereby incorporated by reference in their entireties.

Displacement assays and flow immunosensors useful for carrying out displacement assays are described in: (1) Kusterbeck et al. [1990] “Antibody-Based Biosensor for Continuous Monitoring”, Biosensor Technology, R. P. Buck et al., eds., Marcel Dekker, N.Y., pp. 345-350; Kusterbeck et al. [1990] “A Continuous Flow Immunoassay for Rapid and Sensitive Detection of Small Molecules”, Journal of Immunological Methods, 135:191-7; Ligler et al. [1992] “Drug Detection Using the Flow Inmunosensor”, Biosensor Design and Application, J. Findley et al., eds., American Chemical Society Press, pp. 73-80; and Ogert et al. [1992] “Detection of Cocaine Using the Flow Immunosensor”, Analytical Letters, 25:1999-2019, all of which are incorporated herein by reference in their entireties. Displacement assays and flow immunosensors are also described in U.S. Pat. No. 5,183,740, which is also incorporated herein by reference in its entirety. The displacement immunoassay, unlike most of the competitive immunoassays used to detect small molecules, can generate a positive signal with increasing antigen concentration.

One aspect of the invention allows for the exclusion of Western blots as a diagnostic assay, particularly where the Western blot is a screen of whole cell lysates of C. ruminantium (now E. ruminantium), or related organisms, against immune serum of infected individuals. In another aspect of the invention, peptide, or polypeptide, based diagnostic assays utilize E. ruminantium peptides or polypeptides that have been produce either by chemical peptide synthesis or by recombinant methodologies that utilize non-ehrlichial host cells for the production of peptides or polypeptides.

Also encompassed within the scope of the present invention are fragments and variants of the exemplified polynucleotides and polypeptides. Fragments would include, for example, portions of the exemplified sequences wherein prokaryotic signal sequences have been removed. Examples of the removal of such sequences are given in Example 3. Variants include polynucleotides and/or polypeptides having base or amino acid additions, deletions and substitutions in the sequence of the subject molecule so long as those variants have substantially the same activity or serologic reactivity as the native molecules. Also included are allelic variants of the subject polynucleotides. The polypeptides of the present invention can be used to raise antibodies that are reactive with the polypeptides disclosed herein. The polypeptides and polynucleotides can also be used as molecular weight markers.

Polypeptide fragments, including immunogenic fragments, according to the invention can be any length from at least 5 consecutive amino acids to 1 amino acid less than a full length polypeptide of any given SEQ ID. Thus, for SEQ ID NO:1 (used here as a non-limiting example) the polypeptide fragment can contain any number of consecutive amino acids from 5 to 277. For the sake of brevity, the individual integers between 5 and 277 have not been reproduced herein but are, in fact, specifically contemplated. In one embodiment, the immunogenic fragments of the invention induce protective immunity from disease.

Each fragment as described above can be further specified in terms of its N-terminal (aaₓ) and C-terminal (aa_{y}) positions. For example, every combination of N-terminal to C-terminal fragment, having for example, 5 contiguous amino acids to one amino acid less than the full length polypeptide of a given SEQ ID, are included in the present invention. Any given consecutive amino acid fragment (of a pre-selected length within the full length polypeptide) can be identified using a mathematical formula such as: fragment positioning =(amino acid starting position+(fragment length (in amino acids)−1) or aaₓ−aa_{y}=aaₓ+(fragment length (in amino acids)−1), wherein aaₓ is the position number of the starting amino acid and aay is the position of the ending amino acid.

Thus, a hexameric (6 amino acid) polypeptide fragment of SEQ ID NO:1 could occupy, for example, positions selected from the group consisting of 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 12-24, 20-25, 21-26, 22-27, 23-28, 24-29, 25-30, 26-31, 27-32, 28-33, 29-34, 30-35, 31-36, 32-37 , 33-38, 34-39, 35-40, 36-41, 37-42, 38-43, 39-44, 40-45, 41-46, 42-47, 43-48, 44-49, 45-50, 46-51, 47-52, 48-53, 49-54, 50-55, 51-56, 52-57, 53-58, 54-59, 55-60, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, 75-80,76-81, 77-82, 78-83, 79-84, 80-85, 81-86, 82-87, 83-88, 84-89, 85-90, 86-91, 87-92, 88-93, 89-94, 90-95, 91-96, 92-97, 93-98, 94-99, 95-100, 96-101, 97-102, 98-103, 99-104, 100-105, 101-106, 102-107, 103-108, 104-109, 105-110, 106-111, 107-112, 108-113, 109-114, 110-115, 111-116, 112-117, 113-118, 114-119, 115-120, 116-121, 117-122, 118-123, 119-124, 120-125, 121-126, 123-128, 124-129, 125-130, 126-131, 127-132, 128-133, 129-134, 130-135, 131-136, 132-137, 133-138, 134-139, 135-140, 136-141, 137-142, 138-143, 139-144, 140-145, 141-146, 142-147, 143-148, 144-149, 145-150, 146-151, 147-152, 148-153, 149-154, 150-155, 151-156, 152-157, 153-158, 154-159, 155-160, 156-161, 157-162, 158-163, 159-164, 160-165, 161-166, 162-167, 163-168, 164-169, 165-170, 166-171, 167-172, 168-173, 169-174, 170-175, 171-176, 172-177, 173-178, 174-179, 175-180, 176-181, 177-182, 178-183, 179-184, 180-185, 181-186, 182-187, 183-188, 184-189, 185-190, 186-191, 187-192, 188-193, 189-194, 190-195, 191-196, 192-197, 193-198, 194-199, 195-200, 196-201, 197-202, 198-203, 199-204, 200-205, 201-206, 202-207, 203-208, 204-209, 205-210, 206-211, 207-212, 208-213, 209-214, 210-215, 211-216, 212-217, 213-218, 214-219, 215-220, 216-221, 217-222, 218-223, 219-224, 220-225, 221-226, 222-227, 223-228, 224-229, 225-230, 226-231, 227-232, 228-233, 229-234, 230-235, 231-236, 232-237, 233-238, 234-239, 235-240, 236-241, 237-242, 238-243, 239-244, 240-245, 241-246, 242-247, 243-248, 244-249, 245-250, 246-251, 247-252, 248-253, 249-254, 250-255, 251-256, 252-257, 253-258, 254-259, 255-260, 256-261, 257-262, 258-263, 259-264, 260-265, 261-266, 262-267, 263-268, 264-269, 265-270, 266-271, 267-272, 268-273, 269-274, 270-275, 271-276, 272-277, and 273-278 of SEQ ID NO: 1. A 268 consecutive amino acid fragment could occupy positions selected from the group consisting of 1-268, 2-269, 3-270, 4-271, 5-272, 6-273, 7-274, 8-275, 9-276, 10-277, and 11-278 of SEQ ID NO:1. Similarly, the amino acid positions occupied by all other fragments (of any pre-selected size) of any SEQ ID are included in the present invention and are envisaged as illustrated by these two examples. These fragments are not individually listed solely for the purpose of not unduly lengthening the specification. Furthermore, polynucleotide sequences encoding the envisaged polypeptide fragments are also included in the subject invention and are envisioned as an aspect of the invention.

The present invention also provides for the exclusion of any individual fragment (of any given SEQ ID) specified by N-terminal to C-terminal positions, actual sequence, or of any fragment specified by size (in amino acid residues) as described above. In addition, any number of fragments specified by N-terminal and C-terminal positions, actual sequence, or by size (in amino acid residues) as described above may be excluded as individual species. Further, any number of fragments specified by N-terminal and C-terminal positions or by size (in amino acid residues) as described above may be combined to provide a polypeptide fragment. These types of fragments may, optionally, include polypeptide sequences such as linkers, described below.

Where a claim recites “polypeptide of SEQ ID NO:X, or fragments/immunogenic fragments thereof”, the fragments/immunogenic fragments specifically exclude identical sub-sequences found within other longer naturally occurring prior art polypeptide or protein sequences. This does not include instances where such sub-sequences are a part of a larger molecule specifically modified by the hand of man to enhance the immunogenicity of the fragments of the subject invention. Thus, fragments or immunogenic fragments of a particular SEQ ID specifically exclude, and are not to be considered anticipated, where the fragment is a sub-sequence of another naturally occurring peptide, polypeptide, or protein isolated from a bacterial, viral, reptilian, insect, avian, or mammalian source and is identified in a search of protein sequence databases. The following is an illustration with top sequence identified as “prior art” and the lower sequence exemplifying a fragment of 13 amino acids according to the invention (the “. . . ” elements representing additional amino acids):

| | |
|---|---|
| 5′ . . . Pro Thr Leu Val Thr Leu Ser Val Cys His Phe Gly Ile Glu Leu . . . 3′ | (SEQ ID NO:112) |
| 5′ . . . Leu Val Thr Leu Ser Val Cys His Phe Gly Ile Glu Leu . . . 3′ | (SEQ ID NO:113) |

Thus, as defined by this paragraph, the fragment, as exemplified by the lower sequence, should not be considered to be anticipated by the upper “prior art” sequence.

The fragments and immunogenic fragments of the invention may further contain linkers that facilitate the attachment of the fragments to a carrier molecule for the stimulation of an immune response or diagnostic purposes. The linkers can also be used to attach fragments according to the invention to solid support matrices for use in affinity purification protocols. In this aspect of the invention, the linkers specifically exclude, and are not to be considered anticipated, where the fragment is a subsequence of another peptide, polypeptide, or protein as identified in a search of protein sequence databases as indicated in the preceding paragraph. In other words, the non-identical portions of the other peptide, polypeptide, of protein is not considered to be a “linker” in this aspect of the invention. Non-limiting examples of “linkers” suitable for the practice of the invention include chemical linkers (such as those sold by Pierce, Rockford, Ill.), peptides which allow for the connection of the immunogenic fragment to a carrier molecule (see, for example, linkers disclosed in U.S. Pat. Nos. 6,121,424; 5,843,464; 5,750,352; and 5,990,275, hereby incorporated by reference in their entirety). In various embodiments, the linkers can be up to 50 amino acids in length, up to 40 amino acids in length, up to 30 amino acids in length, up to 20 amino acids in length, up to 10 amino acids in length, or up to 5 amino acids in length. Of course, the linker may be any pre-selected number of amino acids (up to 50 amino acids) in length.

In a specific embodiment, the subject invention concerns a DNA vaccine containing polynucleotides of the invention that are driven by the human cytomegalovirus (HCMV) enhancer-promoter. In a specific example, this vaccine was injected intramuscularly into 8-10 week-old female DBA/2 mice after treating them with 50 μl/muscle of 0.5% bupivacaine 3 days previously. Up to 75% of the VCL1010/MAP1-immunized mice seroconverted and reacted with MAP1 in antigen blots. Splenocytes from immunized mice, but not from control mice immunized with VCL 1010 DNA (plasmid vector, Vical, San Diego) proliferated in response to recombinant MAP1 and E. ruminantium antigens in in vitro lymphocyte proliferation tests. These proliferating cells from mice immunized with VCL 1010/MAP 1 DNA secreted IFN-gamma and IL-2 at concentrations ranging from 610 pg/ml and 152 pg/ml to 1290 pg/ml and 310 pg/ml, respectively. In experiments testing different VCL1010/MAP1 DNA vaccine dose regimens (25-100 μg/dose, 2 or 4 immunizations), survival rates of 23% to 88% (35/92 survivors/total in all VCL1010/MAP1 immunized groups) were observed on challenge with 30LD50 of E. ruminantium. Survival rates of 0% to 3% (1/144 survivors/total in all control groups) were recorded for control mice immunized similarly with VCL1010 DNA or saline. Accordingly, in a specific embodiment, the subject invention concerns the discovery that the gene encoding the MAP1 protein induces protective immunity as a DNA vaccine against rickettsial disease.

The nucleic acid sequences described herein have other uses as well. For example, the nucleic acids of the subject invention can be useful as probes to identify complementary sequences within other nucleic acid molecules or genomes. Such use of probes can be applied to identify or distinguish infectious strains of organisms in diagnostic procedures or in rickettsial research where identification of particular organisms or strains is needed. As is well known in the art, probes can be made by labeling the nucleic acid sequences of interest according to accepted nucleic acid labeling procedures and techniques. A person of ordinary skill in the art would recognize that variations or fragments of the disclosed sequences which can specifically and selectively hybridize to the DNA of rickettsia can also function as a probe. It is within the ordinary skill of persons in the art, and does not require undue experimentation in view of the description provided herein, to determine whether a segment of the claimed DNA sequences is a fragment or variant which has characteristics of the full sequence, e.g., whether it specifically and selectively hybridizes or can confer protection against rickettsial infection in accordance with the subject invention. In addition, with the benefit of the subject disclosure describing the specific sequences, it is within the ordinary skill of those persons in the art to label hybridizing sequences to produce a probe.

Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under moderate to high stringencyconditions by techniques well known in the art, as described, for example, in Keller, G. H., M. M. Manak [1987] DNA Probes, Stockton Press, New York, N.Y., pp. 169-170.

Examples of various stringency conditions are provided herein. Hybridization of immobilized DNA on Southern blots with ³²P-labeled gene-specific probes can be performed by standard methods (Maniatis et a. [1982] Molecular Cloning: A Laboratory Manual, Cold Spring Harbor Laboratory, New York). In general, hybridization and subsequent washes can be carried out under moderate to high stringency conditions that allow for detection of target sequences with homology to the exemplified polynucleotide sequence. For double-stranded DNA gene probes, hybridization can be carried out overnight at 20-25° C. below the melting temperature (Tm) of the DNA hybrid in 6×SSPE, 5×Denhardt's solution, 0.1% SDS, 0.1 mg/ml denatured DNA. The melting temperature is described by the following formula (Beltz et al. [1983] Methods of Enzymology, R. Wu, L. Grossman and K. Moldave [eds.] Academic Press, New York 100:266-285).

Tm=81.5° C.+16.6 Log[Na+]+0.41 (% G+C)−0.61 (% formamide)−600/length of in base pairs.

Washes are typically carried out as follows:

(1) twice at room temperature for 15 minutes in 1×SSPE, 0.1% SDS (low stringency wash);

(2) once at Tm-20° C. for 15 minutes in 0.2×SSPE, 0.1% SDS (moderate stringency wash).

For oligonucleotide probes, hybridization can be carried out overnight at 10-20° C. below the melting temperature (Tm) of the hybrid in 6×SSPE, 5×Denhardt's solution, 0.1% SDS, 0.1 mg/ml denatured DNA. Tm for oligonucleotide probes can be determined by the following formula:

Tm (° C.)=2(number T/A base pairs)+4(number G/C base pairs) (Suggs et al. [1981] ICN-UCLA Symp. Dev. Biol. Using Purified Genes, D. D. Brown [ed.], Academic Press, New York, 23:683-693).

Washes can be carried out as follows:

(1) twice at room temperature for 15 minutes 1×SSPE, 0.1% SDS (low stringency wash;

(2) once at the hybridization temperature for 15 minutes in 1×SSPE, 0.1% SDS (moderate stringency wash).

In general, salt and/or temperature can be altered to change stringency. With a labeled DNA fragment>70 or so bases in length, the following conditions can be used:

| | |
|---|---|
| Low: | 1 or 2X SSPE, room temperature |
| Low: | 1 or 2X SSPE, 42° C. |
| Moderate: | 0.2X or 1X SSPE, 65° C. |
| High: | 0.1X SSPE, 65° C. |

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15 M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al. [1989] Molecular Cloning, A Laboratory Manual, Second Edition, Cold Spring Harbor Press, N.Y., pp. 9.47-9.57; and Ausubel et al. [1989] Current Protocols in Molecular Biology, Green Publishing Associates and Wiley Interscience, N.Y. are incorporated herein in their entirety.

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al. [1989]Molecular Cloning, A Laboratory Manual, Second Edition, Cold Spring Harbor Press, N.Y., pp. 9.47-9.57; and Ausubel et al. [1989]Current Protocols in Molecular Biology, Green Publishing Associates and Wiley Interscience, N.Y. are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

It is also well known in the art that restriction enzymes can be used to obtain functional fragments of the subject DNA sequences. For example, Bal31 exonuclease can be conveniently used for time-controlled limited digestion of DNA (commonly referred to as “erase-a-base” procedures). See, for example, Maniatis et al. [1982] Molecular Cloning: A Laboratory Manual, Cold Spring Harbor Laboratory, New York; Wei et al. [1983] J. Biol. Chem. 258:13006-13512.

In addition, the nucleic acid sequences of the subject invention can be used as molecular weight markers in nucleic acid analysis procedures.

The terms “comprising”, “consisting of” and “consisting essentially of” are defined according to their standard meaning. The terms may be substituted for one another throughout the instant application in order to attach the specific meaning associated with each term.

Following are examples which illustrate procedures for practicing the invention. These examples should not be construed as limiting. All percentages are by weight and all solvent mixture proportions are by volume unless otherwise noted.

### EXAMPLE 1

### Construction of E. ruminantium Genomic Libraries

Organisms were grown in vitro in endothelial cell cultures as described (Byrom, B. and C. E. Yunker [1990] Cytotechnology 4:285-290). DNA was isolated (Byrom, B. and C. E. Yunker [1990] Cytotechnology 4:285-290; Mahan, S. M. etal. [1994] Microbiology 140:2135-2142) from two different strains of E. ruminantium derived either from Zimbabwe (Highway strain) or from Guadeloupe (Gardel strain). Genomic DNA was partially digested with Sau3AI and fragments between 3 and 7 kbp selected by preparative agarose gel electrophoresis. Ligation was performed of size-selected fragments to XhoI digested pGEM-7zf(+) plasmid vector. DNA to be inserted was partially filled in with DATP and dGTP and the digested plasmid vector was partially filled in with dCTP and dTTP. Escherichia coli DH5αF′IQ competent cells were transformed to ampicillin resistance and isolated white colonies were selected in the presence of 5-bromo-4-chloro-3-indolyl b-D-galactoside (X-gal) and isopropyl b-D-thiogalactoside (IPTG). Individual colonies were inoculated into 96-well flat-bottomed microtiter plates containing 0.1 ml L-broth. Following overnight incubation at 37° C., 0.1 ml L-broth containing 30% (v/v) glycerol was added to every well and the plates stored frozen at −70° C. The Highway (hw) and Gardel (gd) strain libraries contained approximately 4000 and 1250 recombinants respectively, of average insert size 3.4+/−1.8 kbp (hw) and 1.8+/−1.6 kb (gd). The genome size of E. ruminantium is approximately 1.6 Mb (de Villiers, E. P., et al. [2000] J. Clin. Microbiol. 38:1967-1970), thus to achieve 99% probability of having a given DNA sequence represented in the hw library would require 2191 colonies (Clarke, L. and J. Carbon [1976] Cell 9:91-99). Although the gd library would not be expected to have a complete representation of the genome of this strain it was included in screening assays for comparison.

### EXAMPLE 2

### Screening of Genomic Libraries with Immune Serum

Individual colonies from each of the more than 5000 microtiter plate wells comprising the two libraries were grown on nitrocellulose filters overlaid on agar plates and then lysed in chloroform vapor. Membranes were washed three times in Tris buffered saline (TBS, 0.02M Tris, 0.5M NaCl pH 7.5), blocked in 1% w/v gelatin for two hours and reacted overnight with a 1:4000 dilution of sheep immune serum (absorbed with E. coli) in TBS containing 1% gelatin. The immune serum was obtained from a sheep infected by intravenous inoculation of cell culture derived E. ruminantium as described below. The membranes were washed three times in TBS containing 1% gelatin and 0.05% (v/v) Tween 20, exposed to 2.5-5 mCi of ¹²⁵I-protein G for one hour at room temperature, washed three times in TBS with 1% gelatin and 0.25% Tween 20, air dried and exposed to X-ray film. E. coli colonies containing vector DNA alone were used as negative controls and colonies containing the map2 gene were positive controls for colony immunoblots.

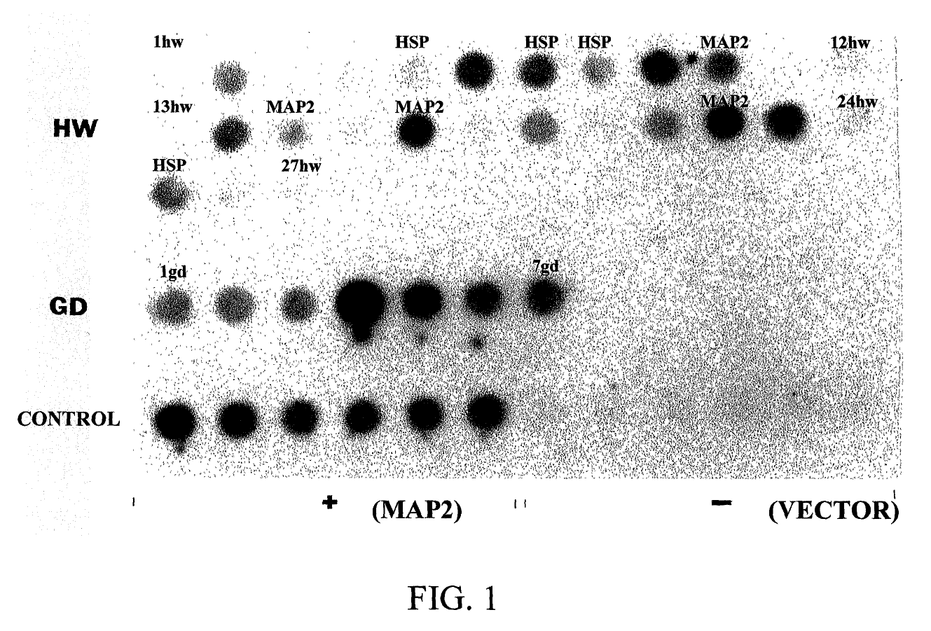

Individual, non-amplified recombinant colonies were screened for expression of antigens reactive with immune serum (1:4000 dilution) from a sheep immune to a Zimbabwe strain of E. ruminantium. Following initial screening of all clones from both libraries, 50 positive clones were obtained. The number of clones was reduced to 34 that were reproducibly positive on multiple re-screening, 27 from the hw (Zimbabwe strain) library (1hw-27hw) and 7 from the gd (Caribbean strain) library (1gd-7gd). These 34 clones were then screened by DNA hybridization to identify those containing the known E. ruminantium antigen genes, map1 (van Vliet, A. H., et al. [1994] Infect. Immun. 62:1451-1456), map2 (Mahan, S. M. et al. [1994] Microbiology 140:2135-2142), and groES/,groEL (Lally, N. C. et al. [1995] Microbiology 141:2091-2100). There were no recombinants containing the map1 gene, 4 that contained map2 and 4 that contained groES/groEL (Table 1 and FIG. 1). Subsequently, the plasmid DNA inserts from each of the 34 clones were tested for cross-hybridization to all other inserts. This allowed 2 other cross-hybridizing plasmid groups to be identified that contained unknown antigen genes (Table 1). Clones 16hw and 26hw cross-hybridized as did clones 18hw, 20hw and 24hw, indicating that the members of these two groups may contain similar antigen genes. Based on the initial characterization, clones were selected for sequencing to avoid re-sequencing known antigen genes or different clones containing the same gene. Clone 2hw was not sequenced because it had a deletion in the vector at the XhoI cloning site, possibly indicating an artefactual recombination. Thirty-four clones inserts were sequenced at both ends to allow clone positioning when the E. ruminantium genomic sequence is completed.

Immune sera were obtained from sheep that had been experimentally infected with E. ruminantium. Sheep 378 and 385 were each infected with 2.7×10⁶ organisms of the Crystal Springs (Zimbabwe) strain. The animals became febrile after 5-7 days and subsequently recovered without treatment. They were re-challenged two times at 3 and 5 months after initial infection without further clinical signs. Sheep 860 was infected with 10×10⁶ organisms of the Mbizi (Zimbabwe) strain, became febrile after 7 days and then recovered. This animal was not re-challenged. Negative sera were obtained from sheep 17 and 77 raised in a heartwater-free area of Zimbabwe.

### EXAMPLE 3

### Screening of Selected Recombinant Colonies by Hybridization

Plasmid DNA was isolated from each of the 34 selected bacterial recombinants and the inserted DNA amplified by PCR using primers AB241 (5′CGGGGTACCGAATTCCTC3′ [SEQ ID NO:114]) and AB242 (5′GCATGCTCCTCTAGACTC3′ [SEQ ID NO:115]) which flank the XhoI site of the pGEM-7zf(+) vector. Amplified DNA was purified from agarose gels and labeled with digoxygenin by random priming (Boehringer Mannheim Corp., Indianapolis, Ind.). DNA probes from each of the 34 selected recombinants were cross-hybridized with plasmid DNA from each of the recombinants in a Mini-blotter 45 (Immunetics, Cambridge, Mass.).

Briefly, plasmid DNA targets, including vector DNA as negative control, were each adjusted to 2 ng/ml in 130 ml of 10×SSC (1.5M NaCl, 0.15 M sodium citrate, pH 7.5) and loaded into the channels of the Mini-blotter 45 containing a 15 cm² piece of pre-wetted, positively charged nylon membrane. The apparatus was incubated at 4° C. with rocking overnight. Following removal of the membrane from the apparatus, DNA was denatured, neutralized and then the membrane rinsed, air dried and u.v. treated to fix the DNA. The membrane was then pre-hybridized (5×Denhardt's solution, 6×SSC, 0.5% (w/v) sodium dodecyl sulfate [SDS], 0.2 mg/ml denatured herring sperm DNA) at 65° C. for 3 hours and placed back in the Mini-blotter but rotated 90 degrees from the original position.

Heat-denatured probes (130 ml) prepared from each of the 34 clones were injected into individual channels of the Mini-blotter, the apparatus wrapped in plastic wrap and placed into a sealed plastic bag containing 1 ml of water to retard evaporation and incubated overnight at 65° C. with rocking. Following hybridization, the membrane was removed and washed to a final stringency of 0.5×SSC, 0.1% SDS at 65° C. Hybridized probe was detected with anti-digoxigenin antibody conjugated to alkaline phosphatase, and chemiluminescence (Boehringer Mannheim). Hybridization of specific gene probes for map1, map2 and groES/groEL of E. ruminantium to the 34 selected recombinants was conducted similarly.

The DNA sequences obtained and their similarities to known sequences in the databases are listed in Table 2. Numerous genes were identified with significant similarity to those of Rickettsia prowazekii as well as genes similar to those encoding outer membrane proteins of other prokaryotes or predicted to encode outer membrane proteins or lipoproteins. Some of these genes have been identified previously in the respective organisms through analysis of antigens that are targets of protective immune responses or involved in pathogenic mechanisms. These include a cell surface protein of Brucella abortus that is a major component of an antigen fraction protective against Brucella in mice (Mayfield, J. E. et al. [1988] Gene 63:1-9) (in clone 1hw), an outer membrane protein involved in iron transport in Pseudomonas aeruginosa that is a target of opsonizing antibodies (Sokol, P. A. [1987] Infection and Immunity 55:2021-2025; Sokol, P. A. and D. E. Woods [1983] Infection and Immunity 40:665-669; Sokol, P. A. and Woods, D. E. [1986] Infection and Immunity 53:621-627) (in clone 4hw) and an outer membrane protein of Coxiella burnetii, coml (Hendrix, L. R. et al. [1993] Infection and Immunity 61:470-477) (in clone 18hw).

Clone 26hw contained a gene of unusual structure encoding 20 tandem repeats of the 9 amino acids VTSSPEGSV (SEQ ID NO:116), and 2 degenerate repeats of the same sequence. Immune sera to E. ruminantium recognized a synthetic peptide containing three of these 9 amino acid repeats in ELISA (FIG. 4).

In addition to these proteins, there were others involved in basic metabolic processes and some of unknown function. Overall, the plasmid insert size was fairly similar in clones selected from the hw library (4299+/−777 bp, range 2,778-6,190) but was diverse in the gd library (1312+/−1284 bp, range 160-3,829). The sequenced plasmids represented a total of about 65 kbp of genomic sequence containing 30 complete and 20 partial genes, none of which have been identified previously in E. ruminantium or in other ehrlichial organisms.

### EXAMPLE 4

### Cell-free Transcription and Translation by Recombinant Plasmid DNAs and Recombinant Expression of Proteins

Coupled in vitro transcription and translation from recombinant plasmid DNAs was performed using the E. coli S30 extract system for circular DNA (Promega, Madison, Wis.). Reactions contained approximately 4 mg plasmid DNA as template, E. coli S30 extract, ³⁵S-methionine, amino acids without methionine, and S30 premix, as described (Promega). Reactions were incubated at 37° C. for two hours and polypeptide products analyzed by electrophoresis on 7.5-17.5% SDS-polyacrylamide gels followed by fluorography.

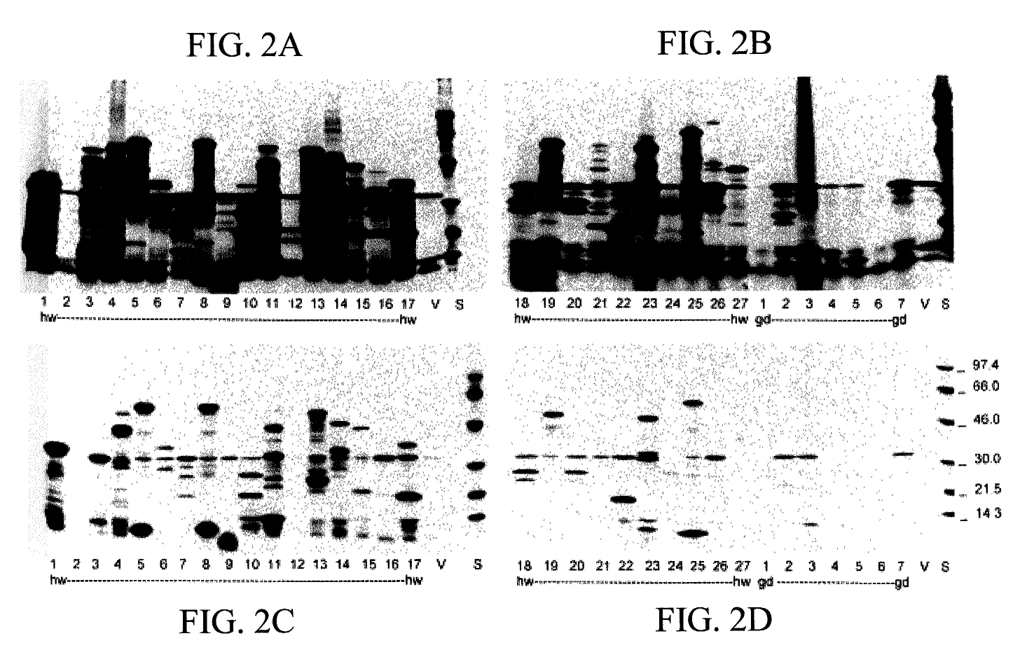

Cell free transcription and translation from recombinant plasmid DNA was used to analyze protein expression in vitro from all 34 selected recombinants and plasmid vector alone as negative control (FIG. 2). Generally, the data correlated with the previous characterization of recombinants by hybridization and sequencing. For example, Table 1 shows that clones 5hw, 7hw, 8hw and 25hw hybridize with a DNA probe containing groES and groEL. GroES and GroEL genes encode polypeptides of 10,462 and 58,697 kD respectively (Lally, N. C. et al. [1995] Microbiology 141:2091-2100). Peptides of the correct size are translated from clones 5hw, 8hw and 25hw (FIG. 2). It is possible that 7hw does not contain the complete genes. A map2 gene probe hybridizes to clones 10hw, 15hw, 17hw and 22hw (Table 1). Map2 encodes a protein of 21 kD on SDS gel electrophoresis (Mahan, S. M. et al. [1994] Microbiology 140:2135-2142). Apeptide of the correct size is synthesized by clones 10hw, 17hw, and 22hw. Recognition of map2 and groEL gene products by immune serum was confirmed by immunoprecipitation of appropriately sized peptides from cell-free synthesis reactions (data not shown).

Table 2 provides a tentative correlation between other protein products of cell-free synthesis reactions and the sequenced genes. 1hw encodes a polypeptide of approximately 37 kD that may correspond to the E. ruminantium analog of a Brucella abortus outer membrane protein. 18hw encodes a 28 kD homolog of the Coxiella burnetii outer membrane protein coml. A protein of identical size is also encoded by the cross-hybridizing clones 20hw and 24hw (Table 1 and FIG. 2).

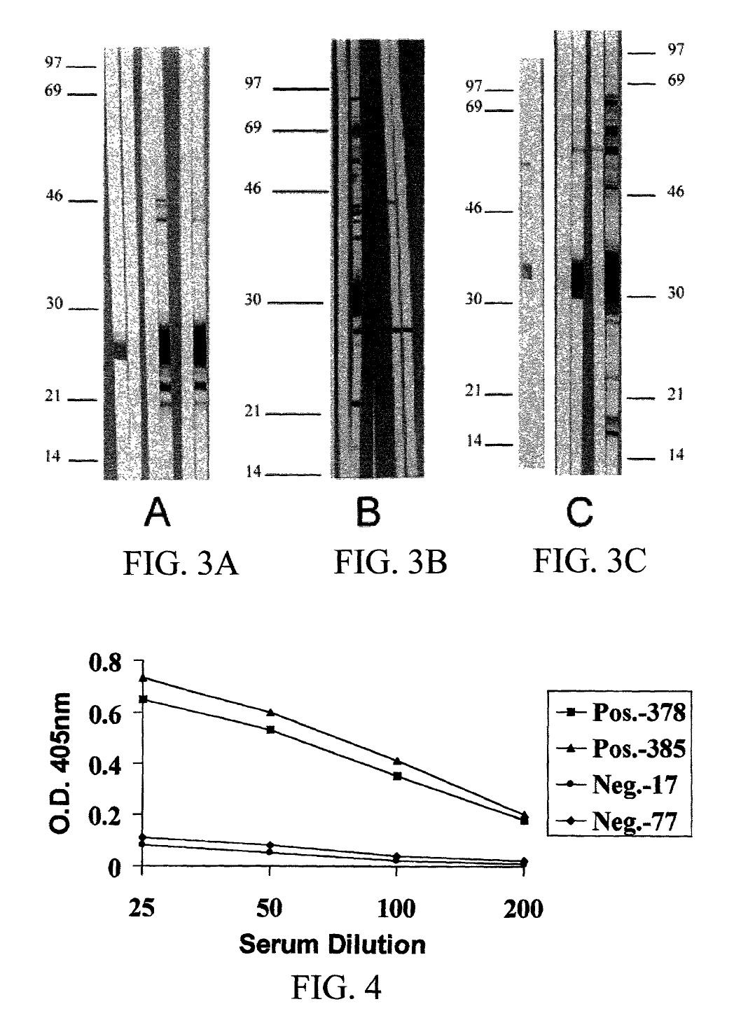

The assignments of these open reading frames to antigen encoding genes were confirmed by immunoprecipitation (data not shown) and subcloning the genes from 1hw and 18hw into the expression vector pGEX-6P-1. Immune serum to E. ruminantium recognized the recombinant proteins in immunoblots (FIG. 3), whereas serum from uninfected animals did not. Antisera to the recombinant analog of Coxiella burnetii coml also recognized E. ruminantium (FIG. 3B). Other tentative assignments of synthesized proteins to their corresponding genes can be made by comparison of Table 2 and FIG. 2, e.g., the major 45 kD band in 4hw to a homolog of Rickettsia prowazekii dihydrolipoamide acetyltransferase, the approximately 55 kD and 26 kD bands in 13 hw to homologs of cytosol aminopeptidase and phosphoribosylamine-glycine ligase.

Selected open reading frames were PCR amplified from recombinant plasmid DNA using Pfu DNA polymerase (Stratagene, La Jolla, Calif.) and cloned into the expression vector pGEX-6P-1 (Pharmacia Biotech, Piscataway, N.J.). Recombinant proteins were purified by affinity chromatography on glutathione sepharose beads followed by elution with reduced glutathione and removal of the fusion partner. Antisera were prepared in rabbits against recombinant proteins using 3 inoculations of 100 mg protein at 2-3 week intervals. The first inoculation contained complete Freund's adjuvant and the next two contained incomplete Freund's adjuvant. Antisera were tested by Western blot against recombinant proteins and E. ruminantium organisms, as described previously (Mahan, S. M. et al. [1993] J. Clin. Microbiol. 31:2729-2737).

### EXAMPLE 5

### DNA Sequence Analysis

DNA sequences were obtained by primer walking. Oligonucleotide primers were designed using OLIGO 5.0 (Molecular Biology Insights, Cascade, Colo.) software and synthesized by Genosys Biotechnologies (The Woodlands, Tex.). Nucleotide sequences were analyzed using the GCG programs (Genetics Computer Group, University of Wisconsin) available through the Biological Computing core facilities of the Interdisciplinary Center for Biotechnology Research at the University of Florida. Predictions of outer membrane proteins were made using PSORT (provided by the Human Genome Center, Institute for Medical Science, University to Tokyo, Japan (Kenta Nakai, Ph.D.)), and of lipoproteins using PROSITE (PCGENE, Intelligenetics, Mountain View, Calif.). Default parameters for the individual computer programs were used in the sequence analysis.

### EXAMPLE 6

### ELISA Assay

96 well plates (Greiner America Inc., Wilmington, Del.) were coated overnight at 4° C. with 100 ml synthetic peptide [VTSSPEGSV (SEQ ID NO:117)]₃ at 10 mg/ml. Plates were washed 5× in 0.2% (v/v) Tween 20 in PBS, pH 7.4, blocked with 5% powdered milk (w/v) in PBS-Tween for 1 hour at 37° C., washed 5× as before, and then reacted with serum diluted in PBS-Tween containing 5% powdered milk. Wells were incubated with diluted serum for 1 hour at 37° C., then washed 5× in PBS-Tween, before addition of a 1:1500 dilution ofperoxidase conjugated rabbit anti-sheep IgG (Kirkegaard and Perry Laboratories, Gaithersburg, Md.). The plates were washed again 5× before reaction with ABTS substrate (2,2′-azinobis(3-ethylbenzthiazoline-sulfonic acid) (Sigma, St. Louis, Mo.). Absorbance was read at 405 nm. Results are depicted in FIG. 4.

### EXAMPLE 7

### Lymphocyte Proliferation Assay

Male Ayrshire calves aged 8-10 months and reared in a heartwater-free area of Kenya were selected for infection. Animals were infected by intravenous inoculation of 5 ml culture supernatant containing 10⁸ viable organisms of E. ruminantium Plumtree (Zimbabwe) strain. These animals were treated on days 2 and 3 of fever by intravenous injection of 10 mg of oxytetracycline per kg of body weight. Establishment of immunity was confirmed by re-challenge with 10⁸culture-derived organisms after a minimum of 4 weeks. Naive control calves were included in the challenge to prove that it was viable.

Recombinant E. Coli lysates were centrifuged at 800×g and sup ematants collected for protein determination. Before use, antigen preparations were sterilized bypassing through 0.2 m filters. Lymphocyte proliferation assays were carried out in triplicate wells of 96-well flat bottomed plates (Costar, Cambridge, Mass.) for 5 days using peripheral blood mononuclear cells (PBMC). Briefly, PBMC were prepared by flotation ofjugular venous blood collected in Alsever's solution on Ficoll-Paque (Pharmacia, Uppsala, Sweden), washed three times in Alsever's solution and re-suspended in RPMI 1640 medium (GIBCO BRL, Grand Island, N.Y.) supplemented with 10% heat-inactivated fetal bovine serum, 2 mM L-glutamine, 50 M 2-mercaptoethanol, 200 IU ofpenicillin per ml, and 150 mg of streptomycin per ml (complete medium). Monocytes were separated from PBMC by adherence to polystyrene. Fifteen ml of a suspension of PBMC in culture medium containing 5×10⁶ cells/ml were placed in a 75 cm² culture flask (Costar) and incubated for 2 hr at 37° C. After incubation, flasks were shaken gently and non-adherent cells removed by pipetting and rinsing with warn (37° C.) RPMI 1640 medium (GIBCO). Adherent cells were removed using PBS containing 0.02% EDTA and washed twice in medium by centrifugation for 10 min at 200 g. All centrifugations were carried out at 4° C. in 10 ml polycarbonate tubes. Monocytes were pulsed overnight with antigens at final concentrations of 10 μg per ml and then washed twice in complete medium by centrifugation. These cells were irradiated and used as antigen presenting cells (APC).

APCs pulsed with appropriate antigens were seeded to wells at a density of 2.5×10⁴ in a volume of 100 ml. PBMC were added at a density of 5×10⁵ cells per well in a volume of 100 ml complete medium. Cultures were incubated for 5 days at 37° C. in a humidified atmosphere of 5% CO₂ in air. Proliferation was assessed by addition of 0.5 μCi [¹²⁵I] iodo-deoxyuridine (Amersham International, Little Chalfont, United Kingdom) to each well and measuring the incorporated radioactivity 8 hr later. Mean counts per minute (cpm) of triplicate samples were determined and results presented as stimulation indices. Positive controls included stimulation with concanavalin A or with recombinant MAP1 protein prepared from E. coli using the pFLAG1 vector (Eastman Kodak, New Haven, Conn.). The negative control was a bacterial lysate from E. coli containing the pGEM-7zf+ vector with no insert DNA.

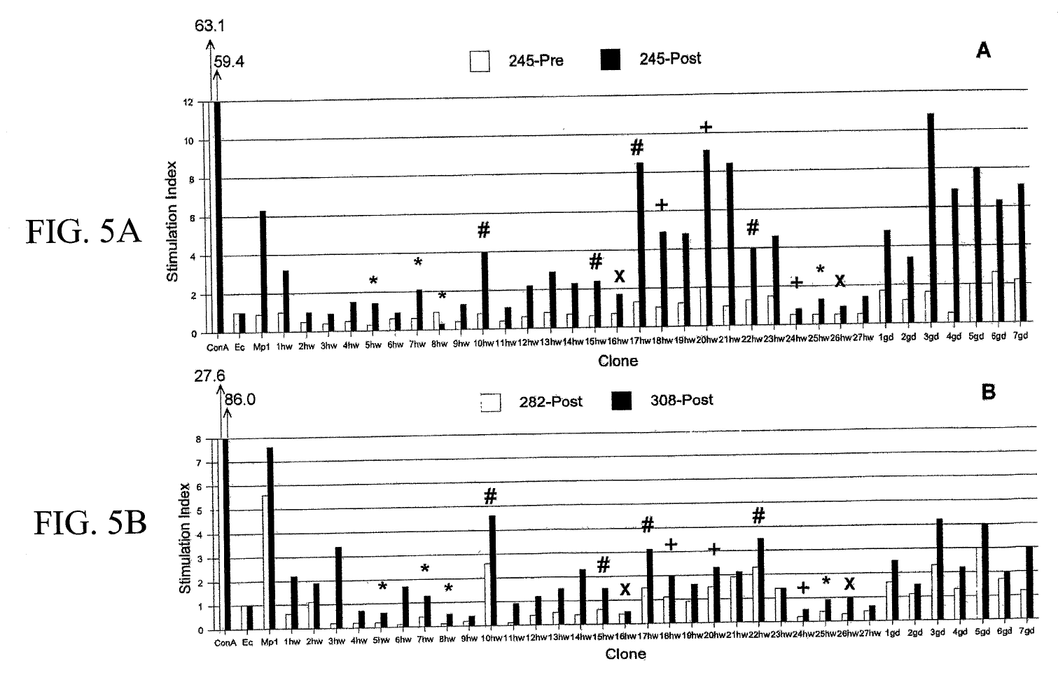

Recombinant antigens encoded by the 34 clones, selected on the basis of antibody recognition, were tested for recognition by PBMC from immune animals. Bacterial lysates from each recombinant were tested for ability to stimulate proliferation. Recombinant MAP1 was used as a positive control as it is known to stimulate proliferative responses in PBMC from immunized animals (Mwangi, D. M. et al. [1998] Ann. N. Y Acad. Sci. 849:372-374); E. coli lysates containing non-recombinant pGEM-7zf(+) vector were used as a negative control (FIG. 5). Although the responses were variable between different infected animals, many clones were recognized by PBMC from immune animals, as indicated by stimulation of proliferative responses. Agreeing with previous data on recognition of MAP2 by T cells from infected animals (Mwangi, D. M. et al. [1998] Ann. N.Y Acad. Sci. 849:372-374), clones 10hw, 17hw and 22hw, which contain the map2 gene (Table 1), were strong inducers of proliferative responses. Clones 18hw and 20hw which encode the 28 kD E. ruminantium analog of the Coxiella burnetii outer membrane protein coml (Table 1 and FIG. 3) were also strongly recognized by immune animals. In addition to these, clones 21hw, 19hw and 3hw induced proliferative responses in some animals but were not recognized by others. This maybe due to the variable abilities of these outbred cattle of different MHC types to respond to the different peptides.

The major peptide synthesized in cell-free reactions by 19hw was approximately 52 kD and by 21hw was 21 kD (FIG. 2). These peptides can be tentatively identified as E. ruminantium homologs of a Rickettsia prowazekii nitrogen assimilation regulatory protein (19hw) and a Pneumocystis carinii folic acid synthesis protein (21hw). Surprisingly, the gd clones generally induced strong proliferative responses, even 5gd which only contained a 160 bp DNA insert.

### EXAMPLE 8

### Immunization and Challenge of Mice

Groups of female DBA/2 mice, 12 to 16 weeks of age, were used in challenge studies. E. coli recombinants were grown overnight, washed 2× and re-suspended in 2.0 ml lysis buffer (50 mM Tris, 5 mM EDTA, 1% NP40, pH 8.0) to a concentration of 1×10¹¹ organisms/ml. Bacteria were frozen, thawed and sonicated and 100 ml bacterial lysate was mixed with 10 mg of the adjuvant Quil A and inoculated subcutaneously into each mouse. For immunization with lysate combinations mice still received a total of 100 ml lysate (from 10¹⁰ organisms) but this was prepared from different bacterial recombinants, e.g., 20 ml lysate from each of 5 different recombinants. Mice received 3 immunizations at 2 week intervals and were then challenged intravenously in the tail vein with E. ruminantium, Highway strain, 4 weeks after the 3rd inoculation. Negative control groups included naive mice and mice that had been immunized with bacterial lysates containing pGEM-7zf(+) vector alone. Groups were challenged in a random order except that naive mice were challenged last to verify adequacy of the challenge. Animals were observed for sickness and death over time.

Recombinant bacterial colonies synthesizing E. ruminantium proteins that were recognized by antibody and PBMC from infected animals were tested for their ability to increase the survival rates of animals challenged with E. ruminantium. DBA/2 mice were each immunized three times with lysates from 10¹⁰ bacteria/injection in Quil A adjuvant and then challenged with a predetermined lethal dose of E. ruminantium organisms. One group of mice was immunized similarly with an E. coli lysate containing non-recombinant vector plasmid as a negative control and a second control group received no immunization.

In the first two experiments (Table 3), groups of mice were immunized with pools containing lysates from 5 different recombinants (the equivalent of 2×10⁹ bacteria from each recombinant). The sufficiency of the challenge dose was confirmed by the survival rate observed in the control groups. In contrast, greater numbers of challenged animals survived in all groups immunized with lysates of recombinant bacteria. In experiment 1, the survival rates of mice immunized with clones 16-20hw and 21-25hw were 60% and 89% respectively, significantly different from the 0 and 10% survival rates in the control groups. In experiment 3 (Table 3), mice were immunized again with a pool of lysates from 21-25hw or with lysates of each recombinant colony comprising the 21-25hw pool. Differences in survival rates were also noted in this experiment.

It should be understood that examples and embodiments described herein are for illustrative purposes only and that various modifications or changes in light thereof will be suggested to persons skilled in the art and are to be included within the spirit and purview of this application.

TABLE 1

| Colony Grouping | Comment |
|---|---|
| 5hw, 7hw, 8hw, 25hw | Hybridize with groES/groEL |
| | probe |
| 10hw, 15hw, 17hw, 22hw | Hybridize with map2 probe |
| 16hw, 26hw | Cross-hybridizing insert DNA |
| 18hw, 20hw, 24hw | Cross-hybridizing insert DNA |

TABLE 2

| | Insert | | Accession | Clone | Entire | | Identity/ | High | Probability |
|---|---|---|---|---|---|---|---|---|---|
| Clone | size | Homologous sequence | number | sequence (nt) | gene | M. wt. | Similarity | Score | (E value) |
| 1hw | 4,360 | Rickettsia prowazekii | PIR:C71673 | 1 to 372 | No | 14,090 | 41/51 | 94 | 2e-19 |
| | | dimethyladenosine | | | | | | | |
| | | transferase | | | | | | | |
| 1hw | | Synechocystis sp. | PIR:S76293 | 611 to 1,333 | Yes | 26,456 | 37/47 | 153 | 5e-37 |
| | | triosephosphate | | | | | | | |
| | | isomerase | | | | | | | |
| 1hw | | Brucella abortus | PIR:IMBKBB | 1,333 to 2,313 | Yes | 35,907 | 31/42 | 171 | 3e-42 |
| | | cell surface protein | | | | | | | |
| | | precursor | | | | | | | |
| 1hw | | Rickettsia prowazekii | PIR:E71711 | 2,611 to 3,366 | Yes | 27,832 | 46/59 | 217 | 3e-56 |
| | | o-sialoglycoprotein | | | | | | | |
| | | endopeptidase | | | | | | | |
| 1hw | | None | | 4,065 to 4,286 | Yes | 8,941 | | | |
| 3hw | 4,055 | Rickettsia prowazekii | SP:Q9ZCN6 | 1 to 1321 | No | 51,512 | 52/65 | 494 | 1e-138 |
| | | valine-tRNA ligase | | | | | | | |
| 3hw | | None | | 1 to 605 | No | 22,109 | | | |
| 3hw | | Thiobacillus ferrooxidans | PIR:E59237 | 3,507 to 4,055 | No | 20,930 | 38/51 | 100 | 2e-20 |
| | | glutamate-cysteine ligase | | | | | | | |
| 4hw | 4,913 | Rickettsia prowazekii | PIR:F71655 | 196 to 714 | Yes | 18,651 | 45/55 | 162 | 5e-40 |
| | | hypothetical ferripyochelin | | | | | | | |
| | | binding protein | | | | | | | |
| 4hw | | Rickettsia prowazekii | GP:CAA14646 | 1,298 to 2,506 | Yes | 44,415 | 53/65 | 397 | 1e-110 |
| | | dihydrolipoamide | | | | | | | |
| | | acetyltransferase | | | | | | | |
| | | component | | | | | | | |
| 4hw | | None | | 3,686 to 4,913 | No | 45,965 | | | |
| 6hw | 6,190 | Rickettsia prowazekii | PIR:B71655 | 1,835 to 2,809 | Yes | 38,331 | 73/79 | 416 | 1e-116 |
| | | ribonucleoside reductase, | | | | | | | |
| | | beta chain | | | | | | | |
| 6hw | | None | | 3,126 to 5,072 | Yes | 72,921 | | | |
| 9hw | 2,778 | Thermotoga maritima | PIR:C72396 | 1 to 551 | No | 20,547 | 31/45 | 84 | 1e-15 |
| | | AraM protein | | | | | | | |
| 9hw | | Rickettsia prowazekii | PIR:G71709 | 1,487 to 2,218 | Yes | 27,030 | 51/64 | 230 | 4e-60 |
| | | ATP synthase A chain | | | | | | | |
| 9hw | | Rickettsia prowazekii | GP:CAA14493 | 2,288 to 2,509 | Yes | 7,774 | 53/62 | 82 | 4e-16 |
| | | ATP synthase C chain | | | | | | | |
| 9hw | | Malawimonas jakobformis | GP:AAG13695 | 2,519 to 2,778 | No | 10,146 | 30/48 | 50 | 6e-06 |
| | | mitochondrion ATP | | | | | | | |
| | | synthase F0 subunit 8 | | | | | | | |
| 11hw | 4,122 | Rickettsia prowazekii | PIR:B71652 | 459 to 2,027 | Yes | 57,773 | 42/50 | 256 | 6e-67 |
| | | iron-sulfur cofactor | | | | | | | |
| | | synthesis protein | | | | | | | |
| 11hw | | Rickettsia prowazekii | PIR:A71652 | 2,099 to 3,340 | Yes | 46,211 | 64/72 | 535 | 1e-151 |
| | | iron-sulfur cofactor | | | | | | | |
| | | synthesis protein | | | | | | | |
| 11hw | | Drosophila melanogaster | SP:Q9ZD61 | 3,375 to 3,788 | Yes | 14,727 | 72/79 | 198 | 2e-50 |
| | | NIFU-like protein | | | | | | | |
| 12hw | 3,814 | Halobacterium sp. | GP:AAC82836 | 144 to 953 | Yes | 28,644 | 29/39 | 87 | 2e-16 |
| | | gas vesicle protein gene | | | | | | | |
| 12hw | | Caulobacter crescentus | GP:AAC72820 | 1177 to 3,814 | No | 93,955 | 35/44 | 231 | 1e-59 |
| | | polar organelle | | | | | | | |
| | | development protein | | | | | | | |
| 12hw | | None | | 1,594 to 3,291 | Yes | 59,939 | | | |
| 12hw | | None | | 2,789 to 3,334 | Yes | 20,061 | | | |
| 13hw | 3,900 | Rickettsia prowazekii | SP:P27888 | 816 to 2,318 | Yes | 54,438 | 46/55 | 397 | 1e-110 |
| | | cytosol aminopeptidase | | | | | | | |
| 13hw | | Zebrafish | GP:AAF71749 | 2,486 to 3,124 | Yes | 23,247 | 46/57 | 180 | 1e-44 |
| | | phosphoribosylamine- | | | | | | | |
| | | glycine ligase | | | | | | | |
| 13hw | | Rickettsia prowazekii | GP:CAA15299 | 3,548 to 3,900 | No | 13,682 | 38/55 | 97 | 8e-20 |
| | | lipoate-protein ligase B | | | | | | | |
| 14hw | 4,369 | None | | 1 to 1,147 | No | 42,556 | | | |
| 14hw | | Helicobacter pylori | PIR:B64703 | 1,354 to 4,119 | Yes | 103,319 | 31/42** | 39 | 0.06 |
| | | membrane protein (lpp*) | | | | | | | |
| 18hw | 3,500 | Coxiella burnetii | GP:BAA20508 | 511 to 1,263 | Yes | 28,127 | 27/37 | 94 | 8e-19 |
| | | outer membrane protein | | | | | | | |
| 18hw | | None | | 1,275 to 3,500 | No | 83,566 | | | |
| 19hw | 4,750 | Rickettsia prowazekii | PIR:B71643 | 1 to 371 | No | 14,622 | 31/47 | 76 | 1e-13 |
| | | probable integrase/ | | | | | | | |
| | | recombinase | | | | | | | |
| 19hw | | Trypanosoma brucei | GP:CAA62581 | 632 to 1,615 | Yes | 35,696 | 35/48 | 186 | 2e-46 |
| | | glycosomal glycerol | | | | | | | |
| | | 3-phosphate | | | | | | | |
| | | dehydrogenase | | | | | | | |
| 19hw | | Rickettsia prowazekii | GP:CAA15010 | 2467 to 3882 | Yes | 53,490 | 49/60 | 399 | 1e-110 |
| | | nitrogen assimilation | | | | | | | |
| | | regulatory protein | | | | | | | |
| 19hw | | Rickettsia prowazekii | PIR:F71679 | 4,154 to 4,750 | No | 22,540 | 26/38 | 71 | 4e-12 |
| | | cell division protein | | | | | | | |
| | | ftsQ | | | | | | | |
| 21hw | 4,544 | Methanococcus jannaschii | PIR:G64457 | 1 to 1,566 | No | 57,998 | 31/43 | 178 | 3e-44 |
| | | phosphoribosylformyl- | | | | | | | |
| | | glycinamidine synthase | | | | | | | |
| 21hw | | Pneumocystis carinii | PIR:S28666 | 2,252 to 2,776 | Yes | 20,044 | 34/45 | 95 | 9e-20 |
| | | folic acid | | | | | | | |
| | | synthesis protein | | | | | | | |
| 21hw | | None | | 3,985 to 4,544 | No | 20,776 | | | |
| 23hw | 4,483 | Escherichia coli | PIR:H64942 | 624 to 1,331 | Yes | 26,641 | 31/47 | 106 | 6e-23 |
| | | hypothetical | | | | | | | |
| | | transmembrane protein | | | | | | | |
| 23hw | | Synechocystis sp. | PIR:S76285 | 2,526 to 2,873 | Yes | 13,311 | 27/39 | 51 | 1e-06 |
| | | ribosome-binding | | | | | | | |
| | | factor A | | | | | | | |
| 23hw | | Rickettsia prowazekii | GP:CAA15001 | 2,870 to 4,483 | No | 58,862 | 49/63 | 496 | 1e-139 |
| | | translation initiation | | | | | | | |
| | | factor ILF-2 | | | | | | | |
| 26hw | 3,829 | Rickettsia prowazekii | PIR:A71706 | 1 to 780 | No | 29,340 | 49/60 | 240 | 3e-63 |
| | | tryptophanyl-tRNA | | | | | | | |
| | | ligase | | | | | | | |
| 26hw | | Macaca mulatta | GP:AAF82403 | 2,361 to 3,416 | Yes | 35,652 | 35/39 | 105 | 8e-22 |
| | | cell surface mucin | | | | | | | |
| | | (tandem repeat) | | | | | | | |
| 26hw | | None | | 3,530 to 3,829 | No | 10,882 | | | |
| | | (except to 26hw orf above) | | | | | | | |
| 27hw | 4,460 | None (omp*) | | 369 to 1,094 | Yes | 27,631 | | | |
| 27hw | | Rickettsia prowazekii | PIR:A71692 | 2,149 to 2,715 | Yes | 21,241 | 51/60 | 152 | 1e-36 |
| | | DNA-3-methyladenine | | | | | | | |
| | | glycosidase | | | | | | | |
| 27hw | | None (lpp*) | | 3,382 to 3,900 | Yes | 19,396 | | | |
| 27hw | | None | | 3,369 to 3,608 | Yes | 8,634 | | | |
| 23hw | | Synechocystis sp. | PIR:S76285 | 2,526 to 2,873 | Yes | 13,311 | 27/39 | 51 | 1e-06 |
| | | ribosome-binding | | | | | | | |
| | | factor A | | | | | | | |
| 2gd | 2,008 | Rickettsia prowazekii | PIR:B71697 | 1 to 348 | No | 13,139 | 23/34 | 41 | 0.001 |
| | | unknown peptide | | | | | | | |
| 3gd | 3,829 | Pyrococcus horikoshi | GP:BAA30538 | 605 to 2,083 | Yes | 54,158 | 33/48 | 218 | 1e-55 |
| | | hypothetical NADH | | | | | | | |
| | | dehydrogenase | | | | | | | |
| | | (ubiquinone) | | | | | | | |
| 3gd | | None | | 2,311 to 2,610 | Yes | 11,460 | | | |
| 3gd | | Pseudomonas putida | GP:AAD17959 | 3,209 to 3,658 | Yes | 16,181 | 36/50 | 89 | 2e-17 |
| | | toluene tolerance protein, | | | | | | | |
| | | ABC transporter (omp*) | | | | | | | |
| 4gd | 226 | Neisseria meningitidis | GP:AAF42385 | 1 to 226 | No | 7,816 | 73/83 | 115 | 4e-26 |
| | | tldD protein | | | | | | | |
| 5gd | 160 | None | 5/6 reading frames open | | | | | | |
| 6gd | 299 | Rickettsia prowazekii | PIR:D71672 | 1 to 299 | No | 11,079 | 33/45 | 44 | 2e-04 |
| | | unknown peptide | | | | | | | |
| 7gd | 2,104 | Ureaplasma urealyticum | GP:AAF30527 | 180 to 2104 | No | 74,059 | 38/51 | | |
| | | DNA ligase | | | | | | | |
| | 381 | 1e-104 | | | | | | | |

TABLE 3

| Immunizing lysate | # surviving/total | % survival |
|---|---|---|
| Experiment 1 | | |
| 1-5hw | 4/10 | 40 |
| 6-10hw | 3/7 | 43 |
| 11-15hw | 2/8 | 25 |
| 16-20hw | 6/10 | 60 |
| 21-25hw | 8/9 | 89 |
| vector | 0/9 | 0 |
| none | 1/10 | 10 |
| Experiment 2 | | |
| 26-27hw + 1-3gd | 5/11 | 45 |
| 4-7gd | 3/10 | 30 |
| vector | 1/10 | 10 |
| none | 1/9 | 11 |
| Experiment 3 | | |
| 21hw | 2/11 | 18 |
| 22hw | 3/12 | 25 |
| 23hw | 2/12 | 17 |
| 24hw | 2/12 | 17 |
| 25hw | 0/10 | 0 |
| 21-25hw | 7/12 | 58 |
| vector | 1/12 | 8 |
| none | 0/10 | 0 |

We claim:

1. An isolated polynucleotide sequence comprising:
a) a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
b) a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
c) a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
d) a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
e) a polynucleotide sequence encoding a polypeptide fragment or variant of a), b), c) or d), wherein said fragment or variant has substantially the same serologic activity as the native polypeptide; or
f) a polynucleotide sequence encoding a polypeptide fragment or variant of a polypeptide encoded by the complement of a), b), c), d), or e), wherein said fragment or variant has substantially the same serologic activity as the native polypeptide.

2. The isolated polynucleotide sequence of claim 1, wherein said polynucleotide sequence further comprises regulatory elements capable of causing the expression of said polynucleotide sequence in expression systems and, optionally, a polynucleotide sequence encoding a heterologous polypeptide sequence.

3. The isolated polynucleotide according to claim 2, wherein said polynucleotide sequence further comprises a vector.

4. The isolated polynucleotide sequence according to claim 3, wherein said vector is a vaccine vector.

5. The isolated polynucleotide sequence according to claim 3, wherein said vector is a replication vector.

6. The isolated polynucleotide sequence of claim 4, further comprising a carrier, pharmaceutical carrier, or adjuvant.

7. A host cell comprising a polynucleotide sequence comprising:
a) a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
b) a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
c) a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
d) a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
e) a polynucleotide sequence encoding a polypeptide fragment or variant of a), b), c) or d), wherein said fragment or variant has substantially the same serologic activity as the native polypeptide; or
f) a polynucleotide sequence encoding a polypeptide fragment or variant of a polypeptide encoded by the complement of a), b), c), d), or e), wherein said fragment or variant has substantially the same serologic activity as the native polypeptide.

8. An isolated polypeptide comprising:
a) a polypeptide encoded by a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53,54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
b) a polypeptide encoded by a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
c) apolypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
d) a polypeptide encoded by a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
e) a polypeptide encoded by a polynucleotide sequence encoding a polypeptide fragment or variant of a), b), c) or d), wherein said fragment or variant has substantially the same serologic activity as the native polypeptide; or
f) a polypeptide encoded by a polynucleotide sequence encoding a polypeptide fragment or variant of a polypeptide encoded by the complement of a), b), c), d), or e), wherein said fragment or variant has substantially the same serologic activity as the native polypeptide.

9. The polypeptide of claim 9, further comprising a carrier, pharmaceutical carrier, or adjuvant.

10. The polypeptide according to claim 9, further comprising a heterologous polypeptide sequence.

11. A method of inducing immunity in an individual comprising the administration of a composition comprising a carrier and:
a)
i) a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
ii) a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
iii) a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
iv) a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
v) a polynucleotide sequence encoding apolypeptide fragment or variant of a), b), c) or d), wherein said fragment or variant has substantially the same serologic activity as the native polypeptide; or
vi) a polynucleotide sequence encoding a polypeptide fragment or variant of a polypeptide encoded by the complement of a), b), c), d), or e), wherein said fragment or variant has substantially the same serologic activity as the native polypeptide; or
b)
i) a polypeptide encoded by a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
ii) a polypeptide encoded by a polynucleotide sequence having at least about 20% to 99.99% identity to a polynucleotide selected from the group consisting of SEQ ID NOs: a polynucleotide sequence selected from the group consisting ofSEQIDNOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
iii) a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
iv) a polypeptide encoded by a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
v) a polypeptide encoded by a polynucleotide sequence encoding a polypeptide fragment or variant of a), b), c) or d), wherein said fragment or variant has substantially the same serologic activity as the native polypeptide; or
vi) a polypeptide encoded by a polynucleotide sequence encoding a polypeptide fragment or variant of a polypeptide encoded by the complement of a), b), c), d), or e), wherein said fragment or variant has substantially the same serologic activity as the native polypeptide.

12. The method according to claim 11, further comprising the administration of an antigen booster.

13. The method according to claim 11, wherein said immunity is protective immunity.

14. The method according to claim 11 a)i), a)ii), a)iii), a)iv), a)v) or a)vi), wherein said polynucleotide sequence further comprises a vaccine vector.

15. The method according to claim 11 a)i), a)ii), a)iii), a)iv), a)v) or a)vi), wherein said polynucleotide sequence further comprises regulatory elements capable of causing the expression of said polynucleotide sequence in expression systems and, optionally, a polynucleotide sequence encoding a heterologous polypeptide sequence.

16. The isolated polynucleotide sequence of claim 4, further comprising a carrier, pharmaceutical carrier, or adjuvant.

17. A method of detecting the presence of Ehrlichia ruminantiuin in a biological sample comprising contacting said sample with:
a)
i) a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
ii) a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
iii) a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107,109, 111, 112, 113, and 116; or
iv) a polynucleotide sequence encoding a polypetide encoded by the complement of SEQ ID NOs:3, 20, 24, 32, 33, 47, 51, 53, 56, 57, 63, 68, 72, 73, 77, 78, 90, 93, 103, and 104; or
v) a polynucleotide sequence encoding a polypeptide fragment or variant of a), b), c) or d), wherein said fragment or variant has substantially the same serologic activity as the native polypeptide; or
vi) a polynucleotide sequence encoding a polypeptide fragment or variant of a polypeptide encoded by the complement of a), b), c), d), or e), wherein said fragment or variant has substantially the same serologic activity as the native polypeptide; or
b)
i) a polypeptide encoded by a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
ii) a polypeptide encoded by a polynucleotide sequence having at least about 20% to 99.99% identity to a polynucleotide selected from the group consisting of SEQ ID NOs: a polynucleotide sequence selected from the group consisting of SEQ ID NOs:2, 3, 4, 8, 10, 12, 15, 17, 19, 20, 21, 23, 24, 25, 27, 29, 31, 32, 33, 34, 36, 38, 39, 41, 43, 45, 46, 47, 48, 51, 52, 53, 54, 56, 57, 58, 59, 61, 63, 64, 65, 67, 68, 69, 70, 72, 73, 74, 75, 77, 78, 79, 81, 82, 84, 85, 87, 88, 90, 92, 93, 94, 95, 96, 98, 100, 101, 103, 104, 105, 106, 108, 110, 114, and 115 or the complements thereof;
iii) a polypeptide selected from the group consisting of SEQ ID NOs:5, 9, 11, 16, 18, 22,26, 28, 30, 35, 37, 40, 42, 44, 55, 60, 62, 66, 71, 76, 80, 83, 86, 89, 97, 99, 102, 107, 109, 111, 112, 113, and 116 or the complement of a polynucleotide sequence encoding a polypeptide selected from the group consisting of SEQ ID NOs:5, 9, 11, 16, 18, 22, 26, 28, 30, 35, 37, 40, 42, 44, 55, 60, 62, 66, 71, 76, 80, 83, 86, 89, 97, 99, 102, 107, 109, 111, 112, 113, and 116; or
iv) a polypeptide encoded by a polynucleotide sequence encoding a polypeptide encoded by the complement of SEQ ID NOs:3, 20, 24, 32, 33, 47, 51, 53, 56, 57, 63, 68, 72, 73, 77, 78, 90, 93, 103, and 104; or
v) a polypeptide encoded by a polynucleotide sequence encoding a polypeptide fragment or variant of a), b), c) or d), wherein said fragment or variant has substantially the same serologic activity as the native polypeptide; or
vi) a polypeptide encoded by a polynucleotide sequence encoding a polypeptide fragment or variant of a polypeptide encoded by the complement of a), b), c), d), or e), wherein said fragment or variant has substantially the same serologic activity as the native polypeptide.


SEQUENCE LISTING (117 sequences)

SEQ ID NO: 1 - protein, 278 aa, Ehrlichia chaffeensis
MNCKKFFITTALVSLMSFLPGISFSDPVQGDNISGNFYVSGKYMPSASHFGMFSAKEEKNPTVALYGLKQDWEGISSSSHNDNHFNNKGYSFKYENNPFLGFAGAIGYSMGGPRVEFEVSYETFDVKNQGNNYKNDAHRYCALGQQDNSGIPKTSKYVLLKSEGLLDISFMLNACYDIINESIPLSPYICAGVGTDLISMFEATNPKISYQGKLGLSYSINPEASVFIGGHFHKVIGNEFRDIPTLKAFVTSSATPDLAIVTLSVCHFGIELGGRFNF

SEQ ID NO: 2 - DNA, 4360 bp, Ehrlichia ruminantium (formerly Cowdria ruminantium)
gatccactttattaaaagtagagttgcaatactataaggtaaatttgctattacttttactggtggtttcgctatatttcgtaaatcaatatctaatgcatctgataatatgaattcatatttaccttgaaattctttaataattttatcatgtattggtaataatctactgtctttctctatagatattaattttttaggattctttttgagtattgaataagtcattgtaccaagtccgggaccaatttcaataattgaaaaattgctaatgtttcctgcataattaactattttatctgtaatatcagttgagtgaatgaaacattggcttaactcttttttaggatttatcatgtaattatcattcataattttttaaattggattattaaattttgtaaatttctaatatcattatactggtaagtagtatgcttatatatacaaattataatttatcagagtattgacttttgtgatatgtgtatataaaatacggaaatgttatagccgacttagcttcaattggtagagcaactgacttgtaatcagtaggttataagttcgagtcttatagtcggcacatcattttactttaagtagttttatgtcattactcatcgttgctaactggaagatgcatggtgatttttttactttttcttcgtttacaaaggagcttagtaaccgtttaattaatatagaagataaagtaaaggtagtattatgcccaccatttattgcgttatctacttatgttaattgtccacataatattaagtttggtggacagaactgttgttatgtatctagtgggaagtacactggagaaattagtgctagtatgttatataactctggatgtagttatgtaatagtgggtcactctgaaaggaggagtacgtttcatgaaactgatcatgatgttaggttaaaagctgaatgtgcgatcgaatcaggattaataccaattatttgtgttggagaaactttactagatagggaaaatggtatgctaaaagatactttattaagtcaatgtagtgaatcttttcctaaaaatggtaagtttatcatagcatatgagccagtatgggcaatagggaacaataaaataccttctactgatgtaataatagaagctttagagattattaggtcatatgattatgtatctgatatcatatatggtggagcagtaaatcatactaatgtaggtgatattgtaagtatcaatcaattgtctggtgttttagttggtagtgctagtttagatatggagagtttttttaatataatatgtagtgctataaatgtgaggcaaagttaatgaagaaaatattggttacgtttttagttgttgttaatgtgttttgtaatgctgccattgcttcaactgactcatcagaagataaacagtatattttaattggtactggttctatgactggagtatattatcctataggaggtagcatatgtaggtttattgcatctgattatggtaatgataataacagcatagtttgttctatatcttctacaactggtagcgtatataatcttaattctatgcgttatgcaaatatggatataggtattattcaatctgatttagagtactatgcatataatggtattggtttatatgaaaaaatgccagcaatgaggcatctaagaatattatcttcattacataaagaatatcttacaattgttgttagggcgaattctaatatatcagttattgatgatataaaaggcaaaagagttaatattggtagtcctggtactggtgtaagaatagcaatgttaaaattgttaaatgaaaaaggatggggaagaaaagattttgctgttatggcagaattaaaatcatcagagcaagctcaagcattatgtgataataaaattgatgtgatggtagatgttgttggacatcctaatgctgcaattcaagaagcagcagcaacttgtgatataaaatttatttctttagatgatgatctcatagataaattacatactaagtatccctattataaaagggatattattagtggtgcgttatacagtaacttacctgatatacaaactgtttcagtaaaagcttctttaataacaactactgaattaagcaatgagttggcctataaagttgttaaatctttggttagccatttacatgaactacatggaattactggagctcttagaaatcttactgtaaaagacatggtacagtcagatattacacctttacatgacggtgcaaaacgttattataaggaaattggagttataaaataaaatattgtggtaagcaatttgctaaaagtagtattagcaatagagacaagctgtgatgaaacagctgttgctgtcgtaagaagtgataagcaagttttatcacataaggtactttcacaaaaagaacatgtagtctatggtggggttgtacctgaaattgcttctcgtgcacatattaactatttatatgacttaacctctcaatctattgaggaatcaggatgtgatttagcagatattgatgctatagcagttacttcaggtccaggtcttattggaggactaattataggtgtaatgatggctaaagctatttccagcgttactaataagcctattattgaggttaatcatctggaagcacatactttgctaatacgaatgtttcatgatattgattttccatttttagtattgatcatatctggcggacattgtcagtttttaatagttcatgatgttggatgttatcaaagattaggttcttctttagatgactcccttggtgaagtatttgataaagtagcaagaatgttgaatttgggatatcctggagggccaattattgaaaaaaaatccataatgggtgatagcaaaagtttttttctaccacgtgcattaatcaatcgtcttggatgtgatttttctttctccggtattaagacggcagtaagaaatattgttgtaaatcaaaaatatatagataatgattttatatgtaatatttcagcttcttttcaagattgtattggtgatatattagtaaacaggattactaatgctattcatatgtcacaagctataaattgtaagattaataagttagtagtaactggaggtgttgcagctaatcacctattacgtaatcgtatatcaatttgtgtaaaagataataattttgaggtgctatatcctccaactgagttatgtactgataatggaattatggttgggtgggctggtattgaaaatttatctaaaggttatgtttctaatttagattttgttccaaaagcaagatggccgttagaaagcataaaaaggtctagttaattattaatacagtagtattttactatacacgattcctattgtatatatttaaaatattgattggctattataaatttttttatttattaaagtactcattttttgcaggaaaaatgtttaatcagtatcaagataatcaagctaacgacaatatttcttattcaggtggaataagaagatttaccagcatactaatagagttagtatttttaatgtttgttttacaaattaatagtggtattttgaaagtaaaatagcatattcatatactaagttattaattaactagattattatgattgttgatatatgtatgcgtatatttaaaaggttaaatatactgatactactaattgatagattgtgtgtatataagaaaaaaaaaaagatggaattgttccttaatatatttatgtctaagtagaaatagtgtgtaaagttgcaatataattggtatttatttctagataaaatttagaatttttattttttttataaagcattcacatagaggtagttaagaaaatgtttaattattaatagtaaaaaggtataaatatggttttgtaagttataatgtaatatcgtgataagattatgtttttttgtgtgattttataaataacaaattgaacagtatataaataccacttttccttaagtaattactactgctaaataaaatcgtagccttttatatgactcttttttactatagaaaattcaccaatctaacaatagtaaataaaaattttttaatttatatgacatttgtatattactataaatcagtatttattaaagttaagaatattaataatgtatttaagtttaaaaaaaacttttttgtaaatagtcatattaatataacttttagcaatataaatattgaattttcagtacttacgtcatactgttaatcctcactataatcatctttatttatcattaataaagagattttttggttttttatgatcatagcttttagatc

SEQ ID NO: 3 - DNA, 372 bp, Ehrlichia ruminantium (formerly Cowdria ruminantium)
  misc_feature (1)..(372): Complement to SEQ ID NO2, nucleotides <1..372 Hypothetical dimethyl adenosine transferase Product=“1hworf1i”
gatccactttattaaaagtagagttgcaatactataaggtaaatttgctattacttttactggtggtttcgctatatttcgtaaatcaatatctaatgcatctgataatatgaattcatatttaccttgaaattctttaataattttatcatgtattggtaataatctactgtctttctctatagatattaattttttaggattctttttgagtattgaataagtcattgtaccaagtccgggaccaatttcaataattgaaaaattgctaatgtttcctgcataattaactattttatctgtaatatcagttgagtgaatgaaacattggcttaactcttttttaggatttatcatgtaattatcattcat

SEQ ID NO: 4 - DNA, 723 bp, Ehrlichia ruminantium (formerly Cowdria ruminantium)
  CDS (1)..(723): Corresponds to SEQ ID NO2, nucleotides 611.. 1333 Hypothetical triosephosphate isomerase Product=“1hworf2”
atgtcattactcatcgttgctaactggaagatgcatggtgatttttttactttttcttcgtttacaaaggagcttagtaaccgtttaattaatatagaagataaagtaaaggtagtattatgcccaccatttattgcgttatctacttatgttaattgtccacataatattaagtttggtggacagaactgttgttatgtatctagtgggaagtacactggagaaattagtgctagtatgttatataactctggatgtagttatgtaatagtgggtcactctgaaaggaggagtacgtttcatgaaactgatcatgatgttaggttaaaagctgaatgtgcgatcgaatcaggattaataccaattatttgtgttggagaaactttactagatagggaaaatggtatgctaaaagatactttattaagtcaatgtagtgaatcttttcctaaaaatggtaagtttatcatagcatatgagccagtatgggcaatagggaacaataaaataccttctactgatgtaataatagaagctttagagattattaggtcatatgattatgtatctgatatcatatatggtggagcagtaaatcatactaatgtaggtgatattgtaagtatcaatcaattgtctggtgttttagttggtagtgctagtttagatatggagagtttttttaatataatatgtagtgctataaatgtgaggcaaagttaa

SEQ ID NO: 5 - protein, 240 aa, Ehrlichia ruminantium (formerly Cowdria ruminantium)
MSLLIVANWKMHGDFFTFSSFTKELSNRLINIEDKVKVVLCPPFIALSTYVNCPHNIKFGGQNCCYVSSGKYTGEISASMLYNSGCSYVIVGHSERRSTFHETDHDVRLKAECAIESGLIPIICVGETLLDRENGMLKDTLLSQCSESFPKNGKFIIAYEPVWAIGNNKIPSTDVIIEALEIIRSYDYVSDIIYGGAVNHTNVGDIVSINQLSGVLVGSASLDMESFFNIICSAINVRQS

SEQ ID NO: 6 - DNA, 981 bp, Ehrlichia ruminantium (formerly Cowdria ruminantium)
  CDS (1)..(981): Corresponds to SEQ ID NO2, nucleotides 1333.. 2313 Hypothetical cell surface protein precursor Product=“1hworf3”
atgaagaaaatattggttacgtttttagttgttgttaatgtgttttgtaatgctgccattgcttcaactgactcatcagaagataaacagtatattttaattggtactggttctatgactggagtatattatcctataggaggtagcatatgtaggtttattgcatctgattatggtaatgataataacagcatagtttgttctatatcttctacaactggtagcgtatataatcttaattctatgcgttatgcaaatatggatataggtattattcaatctgatttagagtactatgcatataatggtattggtttatatgaaaaaatgccagcaatgaggcatctaagaatattatcttcattacataaagaatatcttacaattgttgttagggcgaattctaatatatcagttattgatgatataaaaggcaaaagagttaatattggtagtcctggtactggtgtaagaatagcaatgttaaaattgttaaatgaaaaaggatggggaagaaaagattttgctgttatggcagaattaaaatcatcagagcaagctcaagcattatgtgataataaaattgatgtgatggtagatgttgttggacatcctaatgctgcaattcaagaagcagcagcaacttgtgatataaaatttatttctttagatgatgatctcatagataaattacatactaagtatccctattataaaagggatattattagtggtgcgttatacagtaacttacctgatatacaaactgtttcagtaaaagcttctttaataacaactactgaattaagcaatgagttggcctataaagttgttaaatctttggttagccatttacatgaactacatggaattactggagctcttagaaatcttactgtaaaagacatggtacagtcagatattacacctttacatgacggtgcaaaacgttattataaggaaattggagttataaaataa

SEQ ID NO: 7 - protein, 326 aa, Ehrlichia ruminantium (formerly Cowdria ruminantium)
MKKILVTFLVVVNVFCNAAIASTDSSEDKQYILIGTGSMTGVYYPIGGSICRFIASDYGNDNNSIVCSISSTTGSVYNLNSMRYANMDIGIIQSDLEYYAYNGIGLYEKMPAMRHLRILSSLHKEYLTIVVRANSNISVIDDIKGKRVNIGSPGTGVRIAMLKLLNEKGWGRKDFAVMAELKSSEQAQALCDNKIDVMVDVVGHPNAAIQEAAATCDIKFISLDDDLIDKLHTKYPYYKRDIISGALYSNLPDIQTVSVKASLITTTELSNELAYKVVKSLVSHLHELHGITGALRNLTVKDMVQSDITPLHDGAKRYYKEIGVIK

SEQ ID NO: 8 - DNA, 756 bp, Ehrlichia ruminantium (formerly Cowdria ruminantium)
  CDS (1)..(756): Corresponds to SEQ ID NO2, nucleotides 2611.. 3366 Hypothetical o-sialoglycoprotein endopeptidase Product=“1hworf4”
atgatggctaaagctatttccagcgttactaataagcctattattgaggttaatcatctggaagcacatactttgctaatacgaatgtttcatgatattgattttccatttttagtattgatcatatctggcggacattgtcagtttttaatagttcatgatgttggatgttatcaaagattaggttcttctttagatgactcccttggtgaagtatttgataaagtagcaagaatgttgaatttgggatatcctggagggccaattattgaaaaaaaatccataatgggtgatagcaaaagtttttttctaccacgtgcattaatcaatcgtcttggatgtgatttttctttctccggtattaagacggcagtaagaaatattgttgtaaatcaaaaatatatagataatgattttatatgtaatatttcagcttcttttcaagattgtattggtgatatattagtaaacaggattactaatgctattcatatgtcacaagctataaattgtaagattaataagttagtagtaactggaggtgttgcagctaatcacctattacgtaatcgtatatcaatttgtgtaaaagataataattttgaggtgctatatcctccaactgagttatgtactgataatggaattatggttgggtgggctggtattgaaaatttatctaaaggttatgtttctaatttagattttgttccaaaagcaagatggccgttagaaagcataaaaaggtctagttaa

SEQ ID NO: 9 - protein, 251 aa, Ehrlichia ruminantium (formerly Cowdria ruminantium)
MMAKAISSVTNKPIIEVNHLEAHTLLIRMFHDIDFPFLVLIISGGHCQFLIVHDVGCYQRLGSSLDDSLGEVFDKVARMLNLGYPGGPIIEKKSIMGDSKSFFLPRALINRLGCDFSFSGIKTAVRNIVVNQKYIDNDFICNISASFQDCIGDILVNRITNAIHMSQAINCKINKLVVTGGVAANHLLRNRISICVKDNNFEVLYPPTELCTDNGIMVGWAGIENLSKGYVSNLDFVPKARWPLESIKRSS

SEQ ID NO: 10 - DNA, 222 bp, Ehrlichia ruminantium (formerly Cowdria ruminantium)
  CDS (1)..(222): Corresponds to SEQ ID NO2, nucleotides 4065.. 4286 Product=“1hworf5”
atgactcttttttactatagaaaattcaccaatctaacaatagtaaataaaaattttttaatttatatgacatttgtatattactataaatcagtatttattaaagttaagaatattaataatgtatttaagtttaaaaaaaacttttttgtaaatagtcatattaatataacttttagcaatataaatattgaattttcagtacttacgtcatactgttaa

SEQ ID NO: 11 - protein, 73 aa, Ehrlichia ruminantium (formerly Cowdria ruminantium)
MTLFYYRKFTNLTIVNKNFLIYMTFVYYYKSVFIKVKNINNVFKFKKNFFVNSHINITFSNINIEFSVLTSYC

SEQ ID NO: 12 - DNA, 4913 bp, Ehrlichia ruminantium (formerly Cowdria ruminantium)
gatcttattatatgaacatcctaataattaacattaaaattaatgtgttattagaaagcatgtctaagaaaatatatcttataaatttacagtcaagattgcattgactaagtatttttaatatagatttgtagtaactactatgtaaagtgcattttatataattttaaataaataaataaaaaaggtaagattatgaatatattcaattatatgcagataatgcctaatataagtgttgatgcatttgttgcacctactgctgtaattataggtgatgtttgtgtaaatgacaagtgtagcatttggtataactcagtattacgtggagatgtaggccaaattgttattggtgtaggtactaatattcaagatgggacaataatacatgttgataggaaatatggtaatacgaatattggcaaaaaggttactattgggcatgggtgtatattacatgcttgtgagatacaagattatgtgcttgttggaatgggatctattattatggataacgttgtggttgaaaagaatgcaatggtggctgctggatcattagtggtaagaggtaaagttgtgaaaactggtgaattatgggctggtaggcctgcacaatttttaagaatgttgtctagtgatgaaattaaagagataagtaaatctgctgataactatatagagcttgccagtgattacataactggtaagttgtaattttagttacgctcaaaagataattatctgtattgattgagaatttactagtgtttttttattttatatgaatctatgctatggatgagtctatattctatgtttcaattaattactagatgtagcctaggaagtagggttttgtattatttcattattattgttttaaactatgttagtaatatctgtaaaatgggtagattacttatgtagtatagattttaattaacaattaaagttaaattcctttaatgtgtgttaattctggtgaatacttttattcaagtattttaattacttagtatattcttgatggtgtggtttgttgaaaattacttttctgttataggatggagaataaattcatgttagttatgattggtgaaattagtgatgtataaattataaatcaaatgtgctaatttgttgacaatgggaatttctatgatttatgctatggattatacatcatatgatgttgggtttgtatgtaatttatagtaaataagtagttttttttattatatttcatatgtcattttttgtgttgagtgtagtctaaggaattttttgtggtgcatttatgagtgaagtacaagtaagggctgaaaatcttggtggtgagtcaatattagaagctccaattcgagtttctgttaagattggtgatagtattaagcaaggtgatgtattgtttatcattgaaacggataaaacttctctagaaattgtatctcctgtagatggaacagttagtaaagtatttatagcagatgaagaaattatagaacgtgatcaacttttatgtacaataaatgttggtgaattatcacatattgtccagtctcaaactcaggatcctaaaacagataatggtgatattattaatgatgatattcagacgtttatacagaaaaaagatgctccttctgcagtaaaaattatggcagaaaattcaattgataagaatcagatcaatgggtctggtattggtggaagaattacaaaatctgatgttttagaccacattaatgttgtttcaaaagatcatagtgtgctttctgaacaatgtagtattacttctcatgagaagagagaagaacgtgttaagatgagtaaaattaggcaggtgattgctgcgaggcttaaggagtctcaaaatactgctgcaatattaactacgtttaatgaagtggatatgaagaatgttatggatcttcgtgttcagtatagggagacctttgaaaagaaatatggtgtcaaacttggatttatgtctttttttataaaagcggtagtattagcattaaaagaattaccagtaattaatgctgagatatctggtaatgagattatatataaacattattatgacataggtattgctgtagggacagacaaaggtctagttgttccagtaatgcgtgatgctgataagatgtcttgtgctgagcttgagttaaccttagcttctttaggtaagaaagctagggaagggaaattagaagtttcagatatggctggtgcaacttttactattactaatggtggggtatatggttcattattatctactcctataattaatcctcctcagtctggtattttaggtatgcactctatacaaaaacgaccagtagtagttaatgataattctatagagattagacctatgatgtacattgcattatcttatgatcatagaattgttgatggacaaggtgctgtaacatttttagtaagagttaaacagtatattgaagatccaagtagaatgtttctagaaatataaattatgtatatcggcatttcacaatcatagcagtaattgtttaacatcttgagtttctgtatattttacgaagtaatgtacaaggttgctgtctgacaatattaagttttagtgggttttgtttattggtttattgttaagtaattatagtaaagtataataaatgtaatacagttatagtgctgatatagctaatagtgacgggaatattagtgtttacgcatatataataatagaaattgtatataattcttagtgttaattattttatgttgttatataaaaacgatctcaaaaagtattaatttatataaaaataaaatctaaattttagatggctttattgcaatgtcttttatgttgttatatgagttaatgttaaattacaaagttatgttatagtataaaaataaatgtaacatttctttagctatgcgaaatatataaaactttctttatcactgtgatgtttttgtagagctatctatgaacatattgaaatgttaacattattgaatgtttttcttgtaattatacgttaatgtataggttatttggtaaaaggtatgagctaaatcttgtgttaatataaaaaattaaattgtacggtaatgtaaatttattaagctacatcttttctgtaaaaaatttttgtatttctgtcatctgatgtataaggttacaaatgcagaaatgtaaagtagtttatatgtatgaaggctatggtagcctatataagattgcctacacatattataattatctattgatacttttagtagtactaatatgataagaatatgactaaatttttttattgcatataaaaagtagcagtagttgttgtagagtaatgtagctttttgaatgatatcttataaattaataatattagtacaatatttttataatatttaagaaattaaagaatcaaaatttaaagttattatatcttaattatctaaattttgttgataggtaatcctattttttgaataagaagtgtgtagtttatgattaacaaaacattgttgatgtatttcgtaatagtatattaacagaatttttgtatgtttattttttagaatttaataaaaaattttatatattttaaaaaaaaattaacatcttattgtatttgtattacactggttataaggtgtgttagcggtgttttgtatgttgttgagtaaagataaaaataaaaagaaaaaagatcccaataatcaagaaaatgatgaaaggaatcaaactggtgaatcaggtgttaaacctgaagtaccaaaccagcaaagtattcaagatataggtcagggtgtagtagaaggggcaacagatgctagtgatatgagtggtgttggaagatgtgctttttctgtgactatagagattgaatcatcttcatcaacatctcagccaagtagtagtcttgaaaatatatatatgaggcagggtgctaggccaaaaactaggactcaaagtaaagttgcacagcagagtacaggacaatttcagagtatagggtcacagagtagtttgcctcctgtatttgtaaaacgtatggctgatgtatctttggaaaaggcagaatgtgatacatatatatgtgggactaaaaggcgtagcgatcaaagcacaaggtcaagggaggacttaccttctaggtttgcaaaatgtgcatctgatatatttttaacaaagccacaaaataatgatctagatattcatgatactgataaagagaaattaatacattcattagaagaattagatgttgctctgcctactgagtctggaggtgatcataatgttttatctgatgtcgtatataaaaaatgtgtatccttacaacaaaaatgtggcagttttagacagtttagtaattcttgtctagcaagattaagaggtatgcatatgggatatcttaatttttttatgaaacgattgtttatggctcaaggtaacacattagttatgcgtggggagtacttgcagatgttatcaaatatcactaaacattctgacgaggctgttgttcttgtgaagttaaatcttatgtcacaatatttgcttgcatttggtgcgtatcaggtaagccggtcaatgttaacacagaagcttagtaattctgatttttatgcaattgatattttgttattagaattaatattggtttcgtacaaggagagagtgaatctttattgtgctcaaagagaagttcttaggatgtatgctataatggattataattctggttataatcctaattgtagtaatataaagttttgttatgtaatggtgcaattattccgtgatttattatctgcaagacaaagtatggtgttaggtgatttagatttacaattagttaatttattgattatcagtgttagtattcaaatagatc

SEQ ID NO: 13 - DNA, 519 bp, Ehrlichia ruminantium (formerly Cowdria ruminantium)
  CDS (1)..(519): Corresponds to SEQ ID NO12, nucleotides 196.. 714 Hypothetical ferripyochelin binding protein Product=“4hworf1”
atgaatatattcaattatatgcagataatgcctaatataagtgttgatgcatttgttgcacctactgctgtaattataggtgatgtttgtgtaaatgacaagtgtagcatttggtataactcagtattacgtggagatgtaggccaaattgttattggtgtaggtactaatattcaagatgggacaataatacatgttgataggaaatatggtaatacgaatattggcaaaaaggttactattgggcatgggtgtatattacatgcttgtgagatacaagattatgtgcttgttggaatgggatctattattatggataacgttgtggttgaaaagaatgcaatggtggctgctggatcattagtggtaagaggtaaagttgtgaaaactggtgaattatgggctggtaggcctgcacaatttttaagaatgttgtctagtgatgaaattaaagagataagtaaatctgctgataactatatagagcttgccagtgattacataactggtaagttgtaa

SEQ ID NO: 14 - protein, 172 aa, Ehrlichia ruminantium (formerly Cowdria ruminantium)
MNIFNYMQIMPNISVDAFVAPTAVIIGDVCVNDKCSIWYNSVLRGDVGQIVIGVGTNIQDGTIIHVDRKYGNTNIGKKVTIGHGCILHACEIQDYVLVGMGSIIMDNVVVEKNAMVAAGSLVVRGKVVKTGELWAGRPAQFLRMLSSDEIKEISKSADNYIELASDYITGKL

SEQ ID NO: 15 - DNA, 1209 bp, Ehrlichia ruminantium (formerly Cowdria ruminantium)
  CDS (1)..(1209): Corresponds to SEQ ID NO12, nucleotides 1298.. 2506 Hypothetical dihydrolipoamide acetyltransferase Product=“4hworf2”
atgagtgaagtacaagtaagggctgaaaatcttggtggtgagtcaatattagaagctccaattcgagtttctgttaagattggtgatagtattaagcaaggtgatgtattgtttatcattgaaacggataaaacttctctagaaattgtatctcctgtagatggaacagttagtaaagtatttatagcagatgaagaaattatagaacgtgatcaacttttatgtacaataaatgttggtgaattatcacatattgtccagtctcaaactcaggatcctaaaacagataatggtgatattattaatgatgatattcagacgtttatacagaaaaaagatgctccttctgcagtaaaaattatggcagaaaattcaattgataagaatcagatcaatgggtctggtattggtggaagaattacaaaatctgatgttttagaccacattaatgttgtttcaaaagatcatagtgtgctttctgaacaatgtagtattacttctcatgagaagagagaagaacgtgttaagatgagtaaaattaggcaggtgattgctgcgaggcttaaggagtctcaaaatactgctgcaatattaactacgtttaatgaagtggatatgaagaatgttatggatcttcgtgttcagtatagggagacctttgaaaagaaatatggtgtcaaacttggatttatgtctttttttataaaagcggtagtattagcattaaaagaattaccagtaattaatgctgagatatctggtaatgagattatatataaacattattatgacataggtattgctgtagggacagacaaaggtctagttgttccagtaatgcgtgatgctgataagatgtcttgtgctgagcttgagttaaccttagcttctttaggtaagaaagctagggaagggaaattagaagtttcagatatggctggtgcaacttttactattactaatggtggggtatatggttcattattatctactcctataattaatcctcctcagtctggtattttaggtatgcactctatacaaaaacgaccagtagtagttaatgataattctatagagattagacctatgatgtacattgcattatcttatgatcatagaattgttgatggacaaggtgctgtaacatttttagtaagagttaaacagtatattgaagatccaagtagaatgtttctagaaatataa

SEQ ID NO: 16 - protein, 402 aa, Ehrlichia ruminantium (formerly Cowdria ruminantium)
MSEVQVRAENLGGESILEAPIRVSVKIGDSIKQGDVLFIIETDKTSLEIVSPVDGTVSKVFIADEEIIERDQLLCTINVGELSHIVQSQTQDPKTDNGDIINDDIQTFIQKKDAPSAVKIMAENSIDKNQINGSGIGGRITKSDVLDHINVVSKDHSVLSEQCSITSHEKREERVKMSKIRQVIAARLKESQNTAAILTTFNEVDMKNVMDLRVQYRETFEKKYGVKLGFMSFFIKAVVLALKELPVINAEISGNEIIYKHYYDIGIAVGTDKGLVVPVMRDADKMSCAELELTLASLGKKAREGKLEVSDMAGATFTITNGGVYGSLLSTPIINPPQSGILGMHSIQKRPVVVNDNSIEIRPMMYIALSYDHRIVDGQGAVTFLVRVKQYIEDPSRMFLEI

SEQ ID NO: 17 - DNA, 1227 bp, Ehrlichia ruminantium (formerly Cowdria ruminantium)
  CDS (1)..(1227): Corresponds to SEQ ID NO12, nucleotides 3686.. >4913 Product=“4hworf3i”
atgttgttgagtaaagataaaaataaaaagaaaaaagatcccaataatcaagaaaatgatgaaaggaatcaaactggtgaatcaggtgttaaacctgaagtaccaaaccagcaaagtattcaagatataggtcagggtgtagtagaaggggcaacagatgctagtgatatgagtggtgttggaagatgtgctttttctgtgactatagagattgaatcatcttcatcaacatctcagccaagtagtagtcttgaaaatatatatatgaggcagggtgctaggccaaaaactaggactcaaagtaaagttgcacagcagagtacaggacaatttcagagtatagggtcacagagtagtttgcctcctgtatttgtaaaacgtatggctgatgtatctttggaaaaggcagaatgtgatacatatatatgtgggactaaaaggcgtagcgatcaaagcacaaggtcaagggaggacttaccttctaggtttgcaaaatgtgcatctgatatatttttaacaaagccacaaaataatgatctagatattcatgatactgataaagagaaattaatacattcattagaagaattagatgttgctctgcctactgagtctggaggtgatcataatgttttatctgatgtcgtatataaaaaatgtgtatccttacaacaaaaatgtggcagttttagacagtttagtaattcttgtctagcaagattaagaggtatgcatatgggatatcttaatttttttatgaaacgattgtttatggctcaaggtaacacattagttatgcgtggggagtacttgcagatgttatcaaatatcactaaacattctgacgaggctgttgttcttgtgaagttaaatcttatgtcacaatatttgcttgcatttggtgcgtatcaggtaagccggtcaatgttaacacagaagcttagtaattctgatttttatgcaattgatattttgttattagaattaatattggtttcgtacaaggagagagtgaatctttattgtgctcaaagagaagttcttaggatgtatgctataatggattataattctggttataatcctaattgtagtaatataaagttttgttatgtaatggtgcaattattccgtgatttattatctgcaagacaaagtatggtgttaggtgatttagatttacaattagttaatttattgattatcagtgttagtattcaaatagat

SEQ ID NO: 18 - protein, 409 aa, Ehrlichia ruminantium (formerly Cowdria ruminantium)
MLLSKDKNKKKKDPNNQENDERNQTGESGVKPEVPNQQSIQDIGQGVVEGATDASDMSGVGRCAFSVTIEIESSSSTSQPSSSLENIYMRQGARPKTRTQSKVAQQSTGQFQSIGSQSSLPPVFVKRMADVSLEKAECDTYICGTKRRSDQSTRSREDLPSRFAKCASDIFLTKPQNNDLDIHDTDKEKLIHSLEELDVALPTESGGDHNVLSDVVYKKCVSLQQKCGSFRQFSNSCLARLRGMHMGYLNFFMKRLFMAQGNTLVMRGEYLQMLSNITKHSDEAVVLVKLNLMSQYLLAFGAYQVSRSMLTQKLSNSDFYAIDILLLELILVSYKERVNLYCAQREVLRMYAIMDYNSGYNPNCSNIKFCYVMVQLFRDLLSARQSMVLGDLDLQLVNLLIISVSIQID

SEQ ID NO: 19 - DNA, 6190 bp, Ehrlichia ruminantium (formerly Cowdria ruminantium)
gatctatgtgtatatgcattaacagtagctactcataaaaataataaaaattcctcagacaatcaataattattgataaattaatgtttggtttttctacatctatgtatatactagtataagtttttatttacaaaagacttgctataaatcgcaatacaaaatattgaatacatgaacaaaattgaagaaccaaacattaattgataaagtatattgttattatatacctacaactattatttgtatcatatgacaatttcttattaaataatagtaaaaataatttttatacagcaagatttgatacttaccataataaagtatcacaacaaaaccatataaaataagaaagtagtacacacattataaaaataacgttaatcacccattgcaatgacataataagttacatgggtacacttttgatacttataatagttaataataaaagtagtataataaaaccaaaaccatataaaataagaaagtaccacacattatagaaataataccggtcacccattgcaatgacataataagttacatgggtacacttttgacacttataatagttaataataaaagtagtataacaaaactaaaaccatataaaataagaaagtaccacacattacagaaacaatactggtcacacattgcaatgacataataagttacatgggtacacttttgatacttataatagttaataataaaagtagtataataaaaccaaaaccatataaaataagaaagtaccacacattacagaaacaatactggtcacccattgcaatgacataataagttacatgggtacacttttgatacttataatagttaataataaaagtagtataataaaaccaaaaccatataaaataagaaagtaccacacattacagaaataataccggtcacccattgcaatagcataataagttatattaatacacgttcaattatttatacattgagaaatccataaactcacttagtttagcatatattacatacaaaccttttaaaattatatgggaaatatctccataatattagaaatactataacactcactcatcaaaaaatacaatactagaaaattgtatataaattataaattaaatacctaaacatataaaaacattcatacaccaacagaatgatggtatacgctatattactcttcaaaactatactgaaaggtataataccaactcatcaagaaatgtattatcacaaaattataaccaaactacttttatatatagaaagttcactaatattaaattcaatattatattaataaaaagaaatacactttatgctgattacctattagtaaatcaatatttataagtcatatggtactattaaaagtaaattaacctacatattaaattacattataaaattacttaaaaacaaaaagaactacatatcaggaagaatactatttaaaaacaatctcaaattataaaaaactaatccatatgttcaatttttaaacaatagatctataattacctaaaacaagcaataacattttctaggttatatattatagacccaccaataagacaaaatattaatatacaataacactcattagtaacaaacagtaaatcttgatattttaaagtaaaaatgtttatactattactgaatccttgaagagcatgacaaacgtaaacataagcattaagactaccatataatatatacacattatataaaaaatatttgatacatatagatacaaaaaataaaaacttaacaaacttattaagcatcatatactaatacaataagaactaaagcaatcattttactaatgatcttcaaatgcttcttcccacgttccttcagtagcagcacgtgtatactctgttactctattttcaaagaaattagtgtgctctacaccatttagaatttcatccaaccacaacaaagggttcttatttgcgctgtatataggctctaaatttaactgtattagccttctatttgcaatatatcttatatattctttaacctcctgagcagacaaaccttcaacatcaccacaagcaaatgccaaattaataaactcatcttctaaagttacaataacactacatgcttcatacaattcctttttaagatcatcatcccatatttcataattttcttgaataaaagtattaaataatctaattatcgactcagtatgtaatgtttcatcacgaacagaccaagcaataatttgtcccatgcctttcattttcccaaaacgttgaaaatttagtaaaattgcaaatgaagcaaacaattgtaaaccttctgtaaaggcaccaaaaactgctaaagtttttgctacatgccttttgtcattctttctacactcttcaaaccgttgcatgtagtcatatttctttttcataacctcaaatttcaaaaacgcctgatactctatttctggcatcccaatagtatccaataagtaagaataagcagcaatatgtatagtttccatatttgaaaatgcagataacatcatacatatttccgttggcttaaatatatttgaataatgtttcatataacagttattaacttcgatatcagcctgtgtaaaaaaacgaaaaatttgtactaaaaggttcttttccttatcagaaagtacagtcttccaatcttgaacatcatctgcaagaggcacttcttcaggcaaccagtgtatcctctgctgacataaccatgcatcatacgcccagggataattaaacggtttataaattggtctagaatctagtaatgacatcttaaatgttaccaaaaattacttaagtataacaaagttgcttgcagaactataacaactatttgatagaaaaagcaacttaaaagattaggaatattaaaataatcattagataaaattttaaattattattagtcaatatatggtaaatctagtataaaatagttataaaaattataaatataatatttttctttatataagatactgatattttttataaattatcacttaattattaaaaaaaatatattgcatataaaattatttataattataaattagctaatatttattaatatatttatgtaatgctatgctaggcaattctcaagaatcgagtatctcaagtgaaacacttgaaatatttgcagactctcagattcacataacagaggaacaattaaaaatatatataaagaatcttatagataacttatatgtatataacctactagatcctggaaatgctataccattgtctattatagcaatgctaggtctacattcagattttcattcatttaaaaaagcagtactagatactctttctggatacaaaaattctgtccatagttttcttgcacagtctacaataattgacaggtctgaatctttaagagcagaaccaaatcactgcttatattcattaccacctcttttggataaaagaacttcagaagatatgtggaatgatattaaagaattacacatattatatcaccaatatataattaacgtatctgtagataaaagtactaatgctataagcaatacagtaaatgctccaggcaccaaaacatgttctattaagatatcgtatactaatccattaagacagcatgtacactattttacattaaaaacacttattgaatattacaacactcaacaaacatcattaacaggtcacagatcaattgatgatcaacaagaagctgctgttactttgtttaaagaaacattagaagaaaaattttgcaaaggattaaaaaataaaatattttttaattatgcacaatatttaaaaagtctatttactatcgtaacatcaaatccaaaagtagactatacccttccacaaaatatatatagatattgtgaaacaagaagaatggtaatttcaaaaataacacatgatataattcctatatcagatccaggaactgatatacgtatttattgtgatataccagagtatgtaaccgtattatcagaaacaagtaacattactatatacgggaaagaagtacttggtaaagtttatagcatatatggtacaattataattaaaaacaatatgccacataatgaacgagaaataagctctcgtatatgttctttatttggtcgtgttataatcaatggaagaatacttaatcggaaacatacaatacctagtatatttgaaattaacaaccataacacatacttatcacttaaatataattctatattaacaaaaataacaagcagctctgtaggttccgtaaatgaagaaaaaaaatcacaaatctttgaaatcagtagggatacaattttgaattcaacaaattatcagagaaatatatcaaatttaaaaatagaactacataacccagatgaacaactcacagctactgtcatatcattagatttaaaagatcatccattacctattactaataataatactatacctaatatattaagcctaacagacaatcacgcaacagattcagaattaccaagtgagttttttagtaacaatgttaacccaaaaagtgctggaattacgagaataaaaaatacaattattattgagaaattaactcctacaataggaagatatatgaatgttgccacaaaaaatggaacagtattagataaatatgggatcacagaagtaattattcaaagtaccagaaactttgtaatattattactacatgatgcaaatgttactatagaatgtccattttctggagaaatatttacaaatacaggtaatattacagttattggcccagtaactcacaattctaaacttatttcaaactttggttcagtttatgttggtaatatatctcatcggtcaaatgcattagcaatagataacagccgtattgtatcttcacttgggcatgtcacaatttatggcaaagttagtaaatccaatattactacttctacatcagatgcaatatcaatacataactcaatatcatggtttgataaactaacttcttgtaacaccaaaactttagcatctcgcaaaacataaattatatataagtttcattatggtttgttacctttatatagaaaacagtagattgagtttaaaatatatcagtatcaatatatagtgcatattaagtttaaaaatatatagaacaatatattaaatacaatgttttgatgttttactatgtttatttttaagcttaaaacttagatatgtatttaaccctaaactataaaagtagatgaagtctttgtgctaaatacaaattttatagattattaaaaaagtaaaaaataagatgacataattttactatttatagtaattgaaaatttcaatacctatttatctaacctaaagattttttaattaaaaaaacaatattctttaaagttttaaatatataaagtgtttattaagtacatagtttatattaagctacatgttttttatgcaataaaatcttcacttaacttaaatttctaatttacaaaatgatattttttgagtttttatacctataacattatttatccaacacccatttataggagttataagtttttataataaaaaaaatacaaaagatattctaagattaaagaagaaactacctattatatatgatcctaaaattctatataaatacagaattttcacacttatcttactaaaagaatttaaacacatatccaatcatattcacaaatcatatatgtattataaacaagaatatgcatccaaaaattcaactattctctgtcacactaaattccataatttaaaggtaataattctacaacttgaatactaaataaaatcacttataaaaaatcaatcattaaaccagaaattaaataatttctcaaagtatggtatcacgcctattatagaaaataacatacccatactcaataaagtatatgtaattaatgtataaattatagtaattttattgcgtgtagtagatggtgtaataaagaaaggtataaagttagtagaagtttagcagtatgtgaaagttctttgctttttgtagatagatagtacacacggagttgaagctgagaccttagcaaatgtgtataaggctatagatagcaatcatgagataattcctgtattaaataagatagatttagcgtcttcagatc

SEQ ID NO: 20 - DNA, 975 bp, Ehrlichia ruminantium (formerly Cowdria ruminantium)
  misc_feature (1)..(975): Complement to SEQ ID NO19, nucleotides 1835.. 2809 Hypothetical ribonucleoside reductase, beta chain Product=“6hworf1”
ctaatgatcttcaaatgcttcttcccacgttccttcagtagcagcacgtgtatactctgttactctattttcaaagaaattagtgtgctctacaccatttagaatttcatccaaccacaacaaagggttcttatttgcgctgtatataggctctaaatttaactgtattagccttctatttgcaatatatcttatatattctttaacctcctgagcagacaaaccttcaacatcaccacaagcaaatgccaaattaataaactcatcttctaaagttacaataacactacatgcttcatacaattcctttttaagatcatcatcccatatttcataattttcttgaataaaagtattaaataatctaattatcgactcagtatgtaatgtttcatcacgaacagaccaagcaataatttgtcccatgcctttcattttcccaaaacgttgaaaatttagtaaaattgcaaatgaagcaaacaattgtaaaccttctgtaaaggcaccaaaaactgctaaagtttttgctacatgccttttgtcattctttctacactcttcaaaccgttgcatgtagtcatatttctttttcataacctcaaatttcaaaaacgcctgatactctatttctggcatcccaatagtatccaataagtaagaataagcagcaatatgtatagtttccatatttgaaaatgcagataacatcatacatatttccgttggcttaaatatatttgaataatgtttcatataacagttattaacttcgatatcagcctgtgtaaaaaaacgaaaaatttgtactaaaaggttcttttccttatcagaaagtacagtcttccaatcttgaacatcatctgcaagaggcacttcttcaggcaaccagtgtatcctctgctgacataaccatgcatcatacgcccagggataattaaacggtttataaattggtctagaatctagtaatgacat

SEQ ID NO: 21 - DNA, 1947 bp, Ehrlichia ruminantium (formerly Cowdria ruminantium)
  CDS (1)..(1947): Corresponds to SEQ ID NO19, nucleotides 3126.. 5072 Product = “6hworf2”
atgctaggcaattctcaagaatcgagtatctcaagtgaaacacttgaaatatttgcagactctcagattcacataacagaggaacaattaaaaatatatataaagaatcttatagataacttatatgtatataacctactagatcctggaaatgctataccattgtctattatagcaatgctaggtctacattcagattttcattcatttaaaaaagcagtactagatactctttctggatacaaaaattctgtccatagttttcttgcacagtctacaataattgacaggtctgaatctttaagagcagaaccaaatcactgcttatattcattaccacctcttttggataaaagaacttcagaagatatgtggaatgatattaaagaattacacatattatatcaccaatatataattaacgtatctgtagataaaagtactaatgctataagcaatacagtaaatgctccaggcaccaaaacatgttctattaagatatcgtatactaatccattaagacagcatgtacactattttacattaaaaacacttattgaatattacaacactcaacaaacatcattaacaggtcacagatcaattgatgatcaacaagaagctgctgttactttgtttaaagaaacattagaagaaaaattttgcaaaggattaaaaaataaaatattttttaattatgcacaatatttaaaaagtctatttactatcgtaacatcaaatccaaaagtagactatacccttccacaaaatatatatagatattgtgaaacaagaagaatggtaatttcaaaaataacacatgatataattcctatatcagatccaggaactgatatacgtatttattgtgatataccagagtatgtaaccgtattatcagaaacaagtaacattactatatacgggaaagaagtacttggtaaagtttatagcatatatggtacaattataattaaaaacaatatgccacataatgaacgagaaataagctctcgtatatgttctttatttggtcgtgttataatcaatggaagaatacttaatcggaaacatacaatacctagtatatttgaaattaacaaccataacacatacttatcacttaaatataattctatattaacaaaaataacaagcagctctgtaggttccgtaaatgaagaaaaaaaatcacaaatctttgaaatcagtagggatacaattttgaattcaacaaattatcagagaaatatatcaaatttaaaaatagaactacataacccagatgaacaactcacagctactgtcatatcattagatttaaaagatcatccattacctattactaataataatactatacctaatatattaagcctaacagacaatcacgcaacagattcagaattaccaagtgagttttttagtaacaatgttaacccaaaaagtgctggaattacgagaataaaaaatacaattattattgagaaattaactcctacaataggaagatatatgaatgttgccacaaaaaatggaacagtattagataaatatgggatcacagaagtaattattcaaagtaccagaaactttgtaatattattactacatgatgcaaatgttactatagaatgtccattttctggagaaatatttacaaatacaggtaatattacagttattggcccagtaactcacaattctaaacttatttcaaactttggttcagtttatgttggtaatatatctcatcggtcaaatgcattagcaatagataacagccgtattgtatcttcacttgggcatgtcacaatttatggcaaagttagtaaatccaatattactacttctacatcagatgcaatatcaatacataactcaatatcatggtttgataaactaacttcttgtaacaccaaaactttagcatctcgcaaaacataa

SEQ ID NO: 22 - protein, 648 aa, Ehrlichia ruminantium (formerly Cowdria ruminantium)
MLGNSQESSISSETLEIFADSQIHITEEQLKIYIKNLIDNLYVYNLLDPGNAIPLSIIAMLGLHSDFHSFKKAVLDTLSGYKNSVHSFLAQSTIIDRSESLRAEPNHCLYSLPPLLDKRTSEDMWNDIKELHILYHQYIINVSVDKSTNAISNTVNAPGTKTCSIKISYTNPLRQHVHYFTLKTLIEYYNTQQTSLTGHRSIDDQQEAAVTLFKETLEEKFCKGLKNKIFFNYAQYLKSLFTIVTSNPKVDYTLPQNIYRYCETRRMVISKITHDIIPISDPGTDIRIYCDIPEYVTVLSETSNITIYGKEVLGKVYSIYGTIIIKNNMPHNEREISSRICSLFGRVIINGRILNRKHTIPSIFEINNHNTYLSLKYNSILTKITSSSVGSVNEEKKSQIFEISRDTILNSTNYQRNISNLKIELHNPDEQLTATVISLDLKDHPLPITNNNTIPNILSLTDNHATDSELPSEFFSNNVNPKSAGITRIKNTIIIEKLTPTIGRYMNVATKNGTVLDKYGITEVIIQSTRNFVILLLHDANVTIECPFSGEIFTNTGNITVIGPVTHNSKLISNFGSVYVGNISHRSNALAIDNSRIVSSLGHVTIYGKVSKSNITTSTSDAISIHNSISWFDKLTSCNTKTLASRKT

SEQ ID NO: 23 - DNA, 2778 bp, Ehrlichia ruminantium (formerly Cowdria ruminantium)
gatctacaaataaagtcagcaaaaccactaactataagccttcttggcgcatttacaataatatctacatctatatatatagctttcggtaaatgagcttttaatgactttttgttaccaccactaaatgttatagatgcatttgatgaactatacccattcatagaaggagctgtaggaaatgaaatataatttttatttgtaatgtaactcacatatttacaaagatcatttattgtgccactaccaaaagatactaaaatatcagcatcttgtgatttttctgtaattaattctactaacgtttgagatgcacaatatttttgcaggattataaaatttttaaaagtattaaatacaactttatttaataaagcagcagtatttacatcagctactataaaagcacaattaccatgctgcctaataatatcacatatgttagaactgatttttctatcaatatatatattatctataatattaactactgaacttaattcataaaaatttttatcaagcaaaacttgctttagaaacttatcatacataataaaaaaacaagctatatgctattattgtaacttaatagctaagtttaaaaatctcttatagtaaagtataccaataaaactaaatcttagaaaaaactttctcaaatttaaaatattaattttttttttacaatacgatactagaacacacacatatattagttaactacaaataccagtgtactgctaattcaacatataagtcattgcttataataacattattaattaaaaaataactattctagccagtgttcatcacactatgtcattttacagtagatcacaacttaaagaaacaaaatactattaaaataacacattaaaagcatatcaataatacttaataataaccatcaatgtttataatttatgtaaaataaaaaacttttattcttaatcattacactttatgtatatattacaaatttttgaacaataataaattaaactatcaagaatagttgtcattttaagtttatcacaacataggaaattctatatccctattataagtaacatatatatttaaataatacaatcaacaaataaacacactacaactgataaggttacacctactataaacatatagataaataaaaattcaacataactatcaccaatataagacaaatactgtttttttgaatataggaacattaataacctactataaatgtttctaactttaagtatagtacaaaacaaaatactcatttttaatttatattaaatatatatattttaactacataaattaattaccattataaagaaatatatatacttgagaattatcaaaatatttatcttactatctcaattaatatagttgccttatctacataactgcaattgactaacttatcacagttgatatgattaagaatagcaaaattttactttatatgtgtttatatggagactagatgtcagcaaatcccttagatcagtttaggatttccactatattcaagctacctgatattggcgaatataatattgattttactaatgcctcattatttatggtattatccacatttttaatctccttgtcttgctatgttggattaaggaaagaaagtgttattccgaatccattacaatcaataatagaaattatttatgattttattgtttctacaatagaaagtaatgtaggtaaggaaggattacaatacgtaccattagtttttacaatatttacattcattctagtatgtaatctcttaggtatattaccattagggttcactgtaacaagtcatattgcagtaacatttgcaatatcaatgattgtattcataagtgtaacattcataggatttaaacaccaaggaactcatttccttcatatattgttaccacaaggcacaccaatgtggttagcacctatgatggtcttaattgaattatttgcctattgcgcacgccctgtaagcctatcaatacgactcgctgctaatatgatagctggccatactattatcaaggttatagcaggattcgttataaatatgaatatatttttaacacctttacctatagcattcattataatacttattgggtttgaaatattcgttgcaatcttacaggcatacatttttacagtactcacttgtgtgtacttatcagatgcagtaaataagcactaaattttagcgattgacttcgtataatgatctacttataattcttctagctttttataaaggtaagagtgtatggattctttaaagtttattgctgtagggttaagtgtttttggtatggttgcttctgctttaggggttgcaaatatattttctactatgctaaatggattagcacgtaatcctgaaacagaagataaactaaaaaaatatgtttatactggtgctgctttagttgaagcaatgggtttattttctttcctattagccctattactaatttttgtagcctaatgtagatttatggacacaataccacagttagatatatcttcttatccttctcagtttttttggttttttttatcttttagtgttttgtacattataattagtaaaaatgtgctgccaaagattgaaaatatagtaagaaagaggtataatattataagatgttctattgattctgttaagggtgatttaagccatgcgcagcaagagttagataaacagctgctaaagcttactgcagtacaagcagaagtagatagaattatacgatc

SEQ ID NO: 24 - DNA, 551 bp, Ehrlichia ruminantium (formerly Cowdria ruminantium)
  misc_feature (1)..(551): Complement to SEQ ID NO23, nucleotides <1..551 Hypothetical AraM protein Product = “9hworfli”
gatctacaaataaagtcagcaaaaccactaactataagccttcttggcgcatttacaataatatctacatctatatatatagctttcggtaaatgagcttttaatgactttttgttaccaccactaaatgttatagatgcatttgatgaactatacccattcatagaaggagctgtaggaaatgaaatataatttttatttgtaatgtaactcacatatttacaaagatcatttattgtgccactaccaaaagatactaaaatatcagcatcttgtgatttttctgtaattaattctactaacgtttgagatgcacaatatttttgcaggattataaaatttttaaaagtattaaatacaactttatttaataaagcagcagtatttacatcagctactataaaagcacaattaccatgctgcctaataatatcacatatgttagaactgatttttctatcaatatatatattatctataatattaactactgaacttaattcataaaaatttttatcaagcaaaacttgctttagaaacttatcatacat

SEQ ID NO: 25 - DNA, 732 bp, Ehrlichia ruminantium (formerly Cowdria ruminantium)
  CDS (1)..(732): Corresponds to SEQ ID NO23, nucleotides 1487..      Hypothetical ATP synthase A chain 2218 Product = “9hworf2”
atgtcagcaaatcccttagatcagtttaggatttccactatattcaagctacctgatattggcgaatataatattgattttactaatgcctcattatttatggtattatccacatttttaatctccttgtcttgctatgttggattaaggaaagaaagtgttattccgaatccattacaatcaataatagaaattatttatgattttattgtttctacaatagaaagtaatgtaggtaaggaaggattacaatacgtaccattagtttttacaatatttacattcattctagtatgtaatctcttaggtatattaccattagggttcactgtaacaagtcatattgcagtaacatttgcaatatcaatgattgtattcataagtgtaacattcataggatttaaacaccaaggaactcatttccttcatatattgttaccacaaggcacaccaatgtggttagcacctatgatggtcttaattgaattatttgcctattgcgcacgccctgtaagcctatcaatacgactcgctgctaatatgatagctggccatactattatcaaggttatagcaggattcgttataaatatgaatatatttttaacacctttacctatagcattcattataatacttattgggtttgaaatattcgttgcaatcttacaggcatacatttttacagtactcacttgtgtgtacttatcagatgcagtaaataagcactaa

SEQ ID NO: 26 - protein, 243 aa, Ehrlichia ruminantium (formerly Cowdria ruminantium)
MSANPLDQFRISTIFKLPDIGEYNIDFTNASLFMVLSTFLISLSCYVGLRKESVIPNPLQSIIEIIYDFIVSTIESNVGKEGLQYVPLVFTIFTFILVCNLLGILPLGFTVTSHIAVTFAISMIVFISVTFIGFKHQGTHFLHILLPQGTPMWLAPMMVLIELFAYCARPVSLSIRLAANMIAGHTIIKVIAGFVINMNIFLTPLPIAFIIILIGFEIFVAILQAYIFTVLTCVYLSDAVNKH

SEQ ID NO: 27 - DNA, 222 bp, Ehrlichia ruminantium (formerly Cowdria ruminantium)
  CDS (1)..(222): Corresponds to SEQ ID NO23, nucleotides 2288.. 2509 Hypothetical ATP synthase C chain Product = “9hworf3”
atggattctttaaagtttattgctgtagggttaagtgtttttggtatggttgcttctgctttaggggttgcaaatatattttctactatgctaaatggattagcacgtaatcctgaaacagaagataaactaaaaaaatatgtttatactggtgctgctttagttgaagcaatgggtttattttctttcctattagccctattactaatttttgtagcctaa

SEQ ID NO: 28 - protein, 73 aa, Ehrlichia ruminantium (formerly Cowdria ruminantium)
MDSLKFIAVGLSVFGMVASALGVANIFSTMLNGLARNPETEDKLKKYVYTGAALVEAMGLFSFLLALLLIFVA

SEQ ID NO: 29 - DNA, 261 bp, Ehrlichia ruminantium (formerly Cowdria ruminantium)
  CDS (1)..(261): Corresponds to SEQ ID NO23, nucleotides 2519.. >2778 Hypothetical ATP synthase B chain Product=“9hworf4i”
atggacacaataccacagttagatatatcttcttatccttctcagtttttttggttttttttatcttttagtgttttgtacattataattagtaaaaatgtgctgccaaagattgaaaatatagtaagaaagaggtataatattataagatgttctattgattctgttaagggtgatttaagccatgcgcagcaagagttagataaacagctgctaaagcttactgcagtacaagcagaagtagatagaattatacgatcn

SEQ ID NO: 30 - protein, 87 aa, Ehrlichia ruminantium (formerly Cowdria ruminantium)
  misc_feature (87)..(87): The ′Xaa′ at location 87 stands for Ser.
MDTIPQLDISSYPSQFFWFFLSFSVLYIIISKNVLPKIENIVRKRYNIIRCSIDSVKGDLSHAQQELDKQLLKLTAVQAEVDRIIRX

SEQ ID NO: 31 - DNA, 3814 bp, Ehrlichia ruminantium (formerly Cowdria ruminantium)
gatcgaggccccgcggaagctggaatcgatcatgacccgcgcgtcggtcatggcggccgcgacacgaacctggccgacggtgccgaccgagcgctcgggcgcgggggcggagatggcggggggaggcgggggaagagcctgcatcattccggcactccgggcgcgaaggcgaacaggtccgacggccgcacgaacagttccaggcccatctggatgccgaccagcaggacgatcagcgccagaagcatacgcagtacgtccggccggaaccggcccgacagcctcgccccgtactgggcccccacgaccccaccgaccagcaggatggtcgacaggacgatgtcgacggtctggttgcggcccgcctgaagaatggtggtcatggcggtgacgatgatgatctggaacaggctggtgcccaccacgaccccggccttcatccgcagcacatagagcatggccggcaccagaatgaagccgccgcccacccccatgatggcggacaacacaccggcgaaaccgcccagcgcgaacgggggaatggcgctgatgtagaggcccgagcgggggaagcgcatcttcagcggcaggccatacagccacatcgggcggcggcgttcgcggtgcggcaccacctcgccgcgcacccgcctcaggatctggccgaggctctcgtagagcatcagggtgccgatcgtgccgaggaacagcagataggacaccgctaccgccatgtccgcctgacccagcagacgcagatagcggaacagttccacccccagcagcgcgccgaccacaccacccagggccatgaccccgccgatgcgatagtccaccgccttctggctggtgtagctgatcacgcccgaggtcgaggaggcgacgacgtggctggcctggctggccaccgcaaccgacggcgggattccgagaaagatcaggatcggggccatcaggaagcccccgccgataccgaacagtccggacacgaatccgaccgtcgcgcccagcagaaccaggagcggccagttcaccgaaacctcggcgatcggcagatagatatccaaaggacgcgccttcggctggaactgaggctaggcgccggaaaggcgacgacggaagtggttgtgtcctagacccaccggatggtccccgccacccgatgcgccagaaaagtcagcccgccgcgtccggagcgagcggcgcgacctggaaccgatcagcgtccgcccgggccttaccgcgggccgcggccggcaccgagacccgcaggcgctcgacggccgactgggcgtcgatatccccggcgcgcgatgcgatcaggtaccatttgtaggcttcggtcaggtcgggcgtaatgccgacgtctccggtctcatagagtttggcgacgttgaactggccatcgaccagtccgcgctcggccgagcgtttcagccagaccagggcctctgcacggttctgcgaaccgccgatgccctcgaacaggtacatgccgtagttatacattccgcgggcgtcgccccggtcggccgcgtgacgaacccagacgcgggattcctcaaggtccacggccaggccgttgccaccgtcatgatacaggctggccagatgcagctgggccggggtgtagccggtctgggcggcctgcttcagggtctcgactgcggcggcgtcctcggcgtccagctggaccatcgcctgctggtagagcacctctccgcgggccagatcctcgggcgcaggctcgctcggaacaatggccagggctgcgatcggttgagcggtttcccccgctgtcggcaggccgggaatattcaggcccgatcccgtcagctgtccggtcgcataggccccgccggcggtcagcagcacggcaatggccgaagcgccgagcgccttgcgaaccgtgccaccgtccttgcccgcctgcttttccagccgttcctgaagacgggacttgccgccgcgcttcaggccgaagcccgaacgggccggagcctcgacggcgggagccgccatggccgcacgggcggcgtcgatggtctggcgggtcgaggaggcgcgaccctgggccgccgcagcgcgcagggcccgcggatcgacgaagtccgtttcgccgtcgaaatcgtcatgaccgccgtcaaaggccgacggggtcctgtttccgggcgcggtcgcctccaaggcatccgagacgtccgcaccgccgaagccctggccgaaagcgaccggagcagccgggatctgaaccggcggagcatcgaccggggccggctgggtggccgcgaacggtggcggcgtgaaggcggggatctgcggcggcgggccgacttcctcggccagcgaggtcatggtgggtcggctgccgaacggcatcggagcggcgacgggtacctctgcggtcggtgcgaggtcctcatccacgcgggggccgcgcatgaccgcgtcgaagatggcgtcggcggtcaggacctcgggggtcgcgcgctcgacagggaagggcgcggggtccggctcgggcggagccagattcgcggaccaggcctgctgctcggcatagatcgggtcgtcgaagactgcgtccgagaaaggcgcatcggggaaggctgtgtccggaaaggccgcggcccgccaatcgggttccggcacggcggcgaccgcagtcggaacttcggcccggcgttcgatattgccacgcgcctcggcgatcagggccgccgtgcgctcttccgacatccgcatccgctcggccagttcgccggaagcgcggtcgtagccctgctcgatgcgggcggaactgtcggcgagacggcggccgatatcgtccagcgcctgctgcgaccgccgctccgactgggcgatgcgttcgctcagccggtcggagatacgggtgatctccccgcccagtttttccagggccagggcatgccggtcgtcggtcgcgatcagtcgctgttcgatgccctgggcgaagcggcccatgtcggtctcgaccttgcggctcagctgttcgaaacgggccggaacctcggtctcgatcgactggacccgaccattcaggttctgggcgatgcgcagaacctcgcgccccatcgcctcgaccgccttggcggagcgttcctccgacgccttgacctgctcgccgatggccaggaccgcgcgctcgatccggtccatccggccttcggtttcggctgtatcgagccgccgcatcatctcggcgcgattggcttcaaccgaccggctcagggtctcggcgagcttttcgaagcgggcggcctcgcgtgagccctcgggctcgacccgggactcggcggcgcgcagccgctgatcgagaccggcgaatgaagcctgcaggctgcgcagggcgtcggcggtacggttctgggcctgatccagccgctggccgacctgggccagaagctcggtaccgacacccgggccggcggccttttcgaccgcgtccatccgctggcgcagttcattgacgcccgagcgttgacgctcctcgatatcgtagaggcggcccgccagcgcccccatggtggtctcgaccttgctgaaggtctcggcggtctgagggcccgtttcctgctcgaaccggcgcagacgcttgtgcccctcgcgcaactcctcggcgatgtcgtcaatgcgacgaccataggccctgcccgcgtcgtcctgcccctccagtcggcggacaaggcccgagacggcctgatcgaccccctggatggcgacggttgagcgacgctcggcggcttcgagccgggccgcgatggcatcgaccgaggccccgaggcgctggatc

SEQ ID NO: 32 - DNA, 810 bp, Ehrlichia ruminantium (formerly Cowdria ruminantium)
  misc_feature (1)..(810): Complement to SEQ ID NO31, nucleotides 144.. 953 Similar to gas vesicle protein Product = “12hworf1”
tcattccggcactccgggcgcgaaggcgaacaggtccgacggccgcacgaacagttccaggcccatctggatgccgaccagcaggacgatcagcgccagaagcatacgcagtacgtccggccggaaccggcccgacagcctcgccccgtactgggcccccacgaccccaccgaccagcaggatggtcgacaggacgatgtcgacggtctggttgcggcccgcctgaagaatggtggtcatggcggtgacgatgatgatctggaacaggctggtgcccaccacgaccccggccttcatccgcagcacatagagcatggccggcaccagaatgaagccgccgcccacccccatgatggcggacaacacaccggcgaaaccgcccagcgcgaacgggggaatggcgctgatgtagaggcccgagcgggggaagcgcatcttcagcggcaggccatacagccacatcgggcggcggcgttcgcggtgcggcaccacctcgccgcgcacccgcctcaggatctggccgaggctctcgtagagcatcagggtgccgatcgtgccgaggaacagcagataggacaccgctaccgccatgtccgcctgacccagcagacgcagatagcggaacagttccacccccagcagcgcgccgaccacaccacccagggccatgaccccgccgatgcgatagtccaccgccttctggctggtgtagctgatcacgcccgaggtcgaggaggcgacgacgtggctggcctggctggccaccgcaaccgacggcgggattccgagaaagatcaggatcggggccat

SEQ ID NO: 33 - DNA, 2638 bp, Ehrlichia ruminantium (formerly Cowdria ruminantium)
  misc_feature (1)..(2638): Complement to SEQ ID NO31, nucleotides 1177.. >3814 Hypothetical polar organelle development protein Product = “12hworf2i”
tcagcccgccgcgtccggagcgagcggcgcgacctggaaccgatcagcgtccgcccgggccttaccgcgggccgcggccggcaccgagacccgcaggcgctcgacggccgactgggcgtcgatatccccggcgcgcgatgcgatcaggtaccatttgtaggcttcggtcaggtcgggcgtaatgccgacgtctccggtctcatagagtttggcgacgttgaactggccatcgaccagtccgcgctcggccgagcgtttcagccagaccagggcctctgcacggttctgcgaaccgccgatgccctcgaacaggtacatgccgtagttatacattccgcgggcgtcgccccggtcggccgcgtgacgaacccagacgcgggattcctcaaggtccacggccaggccgttgccaccgtcatgatacaggctggccagatgcagctgggccggggtgtagccggtctgggcggcctgcttcagggtctcgactgcggcggcgtcctcggcgtccagctggaccatcgcctgctggtagagcacctctccgcgggccagatcctcgggcgcaggctcgctcggaacaatggccagggctgcgatcggttgagcggtttcccccgctgtcggcaggccgggaatattcaggcccgatcccgtcagctgtccggtcgcataggccccgccggcggtcagcagcacggcaatggccgaagcgccgagcgccttgcgaaccgtgccaccgtccttgcccgcctgcttttccagccgttcctgaagacgggacttgccgccgcgcttcaggccgaagcccgaacgggccggagcctcgacggcgggagccgccatggccgcacgggcggcgtcgatggtctggcgggtcgaggaggcgcgaccctgggccgccgcagcgcgcagggcccgcggatcgacgaagtccgtttcgccgtcgaaatcgtcatgaccgccgtcaaaggccgacggggtcctgtttccgggcgcggtcgcctccaaggcatccgagacgtccgcaccgccgaagccctggccgaaagcgaccggagcagccgggatctgaaccggcggagcatcgaccggggccggctgggtggccgcgaacggtggcggcgtgaaggcggggatctgcggcggcgggccgacttcctcggccagcgaggtcatggtgggtcggctgccgaacggcatcggagcggcgacgggtacctctgcggtcggtgcgaggtcctcatccacgcgggggccgcgcatgaccgcgtcgaagatggcgtcggcggtcaggacctcgggggtcgcgcgctcgacagggaagggcgcggggtccggctcgggcggagccagattcgcggaccaggcctgctgctcggcatagatcgggtcgtcgaagactgcgtccgagaaaggcgcatcggggaaggctgtgtccggaaaggccgcggcccgccaatcgggttccggcacggcggcgaccgcagtcggaacttcggcccggcgttcgatattgccacgcgcctcggcgatcagggccgccgtgcgctcttccgacatccgcatccgctcggccagttcgccggaagcgcggtcgtagccctgctcgatgcgggcggaactgtcggcgagacggcggccgatatcgtccagcgcctgctgcgaccgccgctccgactgggcgatgcgttcgctcagccggtcggagatacgggtgatctccccgcccagtttttccagggccagggcatgccggtcgtcggtcgcgatcagtcgctgttcgatgccctgggcgaagcggcccatgtcggtctcgaccttgcggctcagctgttcgaaacgggccggaacctcggtctcgatcgactggacccgaccattcaggttctgggcgatgcgcagaacctcgcgccccatcgcctcgaccgccttggcggagcgttcctccgacgccttgacctgctcgccgatggccaggaccgcgcgctcgatccggtccatccggccttcggtttcggctgtatcgagccgccgcatcatctcggcgcgattggcttcaaccgaccggctcagggtctcggcgagcttttcgaagcgggcggcctcgcgtgagccctcgggctcgacccgggactcggcggcgcgcagccgctgatcgagaccggcgaatgaagcctgcaggctgcgcagggcgtcggcggtacggttctgggcctgatccagccgctggccgacctgggccagaagctcggtaccgacacccgggccggcggccttttcgaccgcgtccatccgctggcgcagttcattgacgcccgagcgttgacgctcctcgatatcgtagaggcggcccgccagcgcccccatggtggtctcgaccttgctgaaggtctcggcggtctgagggcccgtttcctgctcgaaccggcgcagacgcttgtgcccctcgcgcaactcctcggcgatgtcgtcaatgcgacgaccataggccctgcccgcgtcgtcctgcccctccagtcggcggacaaggcccgagacggcctgatcgaccccctggatggcgacggttgagcgacgctcggcggcttcgagccgggccgcgatggcatcgaccgaggccccgaggcgctggatc

SEQ ID NO: 34 - DNA, 1698 bp, Ehrlichia ruminantium (formerly Cowdria ruminantium)
  CDS (1)..(1698): Corresponds to SEQ ID NO31, nucleotides 1594.. 3291 Product = “12hworf3”
atgatacaggctggccagatgcagctgggccggggtgtagccggtctgggcggcctgcttcagggtctcgactgcggcggcgtcctcggcgtccagctggaccatcgcctgctggtagagcacctctccgcgggccagatcctcgggcgcaggctcgctcggaacaatggccagggctgcgatcggttgagcggtttcccccgctgtcggcaggccgggaatattcaggcccgatcccgtcagctgtccggtcgcataggccccgccggcggtcagcagcacggcaatggccgaagcgccgagcgccttgcgaaccgtgccaccgtccttgcccgcctgcttttccagccgttcctgaagacgggacttgccgccgcgcttcaggccgaagcccgaacgggccggagcctcgacggcgggagccgccatggccgcacgggcggcgtcgatggtctggcgggtcgaggaggcgcgaccctgggccgccgcagcgcgcagggcccgcggatcgacgaagtccgtttcgccgtcgaaatcgtcatgaccgccgtcaaaggccgacggggtcctgtttccgggcgcggtcgcctccaaggcatccgagacgtccgcaccgccgaagccctggccgaaagcgaccggagcagccgggatctgaaccggcggagcatcgaccggggccggctgggtggccgcgaacggtggcggcgtgaaggcggggatctgcggcggcgggccgacttcctcggccagcgaggtcatggtgggtcggctgccgaacggcatcggagcggcgacgggtacctctgcggtcggtgcgaggtcctcatccacgcgggggccgcgcatgaccgcgtcgaagatggcgtcggcggtcaggacctcgggggtcgcgcgctcgacagggaagggcgcggggtccggctcgggcggagccagattcgcggaccaggcctgctgctcggcatagatcgggtcgtcgaagactgcgtccgagaaaggcgcatcggggaaggctgtgtccggaaaggccgcggcccgccaatcgggttccggcacggcggcgaccgcagtcggaacttcggcccggcgttcgatattgccacgcgcctcggcgatcagggccgccgtgcgctcttccgacatccgcatccgctcggccagttcgccggaagcgcggtcgtagccctgctcgatgcgggcggaactgtcggcgagacggcggccgatatcgtccagcgcctgctgcgaccgccgctccgactgggcgatgcgttcgctcagccggtcggagatacgggtgatctccccgcccagtttttccagggccagggcatgccggtcgtcggtcgcgatcagtcgctgttcgatgccctgggcgaagcggcccatgtcggtctcgaccttgcggctcagctgttcgaaacgggccggaacctcggtctcgatcgactggacccgaccattcaggttctgggcgatgcgcagaacctcgcgccccatcgcctcgaccgccttggcggagcgttcctccgacgccttgacctgctcgccgatggccaggaccgcgcgctcgatccggtccatccggccttcggtttcggctgtatcgagccgccgcatcatctcggcgcgattggcttcaaccgaccggctcagggtctcggcgagcttttcgaagcgggcggcctcgcgtga

SEQ ID NO: 35 - protein, 565 aa, Ehrlichia ruminantium (formerly Cowdria ruminantium)
MIQAGQMQLGRGVAGLGGLLQGLDCGGVLGVQLDHRLLVEHLSAGQILGRRLARNNGQGCDRLSGFPRCRQAGNIQARSRQLSGRIGPAGGQQHGNGRSAERLANRATVLARLLFQPFLKTGLAAALQAEARTGRSLDGGSRHGRTGGVDGLAGRGGATLGRRSAQGPRIDEVRFAVEIVMTAVKGRRGPVSGRGRLQGIRDVRTAEALAESDRSSRDLNRRSIDRGRLGGRERWRREGGDLRRRADFLGQRGHGGSAAERHRSGDGYLCGRCEVLIHAGAAHDRVEDGVGGQDLGGRALDREGRGVRLGRSQIRGPGLLLGIDRVVEDCVRERRIGEGCVRKGRGPPIGFRHGGDRSRNFGPAFDIATRLGDQGRRALFRHPHPLGQFAGSAVVALLDAGGTVGETAADIVQRLLRPPLRLGDAFAQPVGDTGDLPAQFFQGQGMPVVGRDQSLFDALGEAAHVGLDLAAQLFETGRNLGLDRLDPTIQVLGDAQNLAPHRLDRLGGAFLRRLDLLADGQDRALDPVHPAFGFGCIEPPHHLGAIGFNRPAQGLGELFEAGGLA

SEQ ID NO: 36 - DNA, 546 bp, Ehrlichia ruminantium (formerly Cowdria ruminantium)
  CDS (1)..(546): Corresponds to SEQ ID NO31, nucleotides 2789.. 3334 Product = “12hworf4”
atgcgggcggaactgtcggcgagacggcggccgatatcgtccagcgcctgctgcgaccgccgctccgactgggcgatgcgttcgctcagccggtcggagatacgggtgatctccccgcccagtttttccagggccagggcatgccggtcgtcggtcgcgatcagtcgctgttcgatgccctgggcgaagcggcccatgtcggtctcgaccttgcggctcagctgttcgaaacgggccggaacctcggtctcgatcgactggacccgaccattcaggttctgggcgatgcgcagaacctcgcgccccatcgcctcgaccgccttggcggagcgttcctccgacgccttgacctgctcgccgatggccaggaccgcgcgctcgatccggtccatccggccttcggtttcggctgtatcgagccgccgcatcatctcggcgcgattggcttcaaccgaccggctcagggtctcggcgagcttttcgaagcgggcggcctcgcgtgagccctcgggctcgacccgggactcggcggcgcgcagccgctga

SEQ ID NO: 37 - protein, 181 aa, Ehrlichia ruminantium (formerly Cowdria ruminantium)
MRAELSARRRPISSSACCDRRSDWAMRSLSRSEIRVISPPSFSRARACRSSVAISRCSMPWAKRPMSVSTLRLSCSKRAGTSVSIDWTRPFRFWAMRRTSRPIASTALAERSSDALTCSPMARTARSIRSIRPSVSAVSSRRIISARLASTDRLRVSASFSKRAASREPSGSTRDSAARSR

SEQ ID NO: 38 - DNA, 3901 bp, Ehrlichia ruminantium (formerly Cowdria ruminantium)
  misc_feature (3901)..(3901): n = a, c, g, or t
gatcaaaatatgattctttatattatgtcagtttgttaagattcaaatgttcttatatcaataagattatttctactttttagttgtactgctaaattttggtaaacagtattcagtatactaaatttaatgaattattttattagataatgtaagagaaaagatatagtggtttaatttatatatttggaaaatatgaaagcgataaatatgataatatttgtatacagttattttatttaattagctaatacataagaaatagctatcactatgtttcaaaaaggtaattgtgataacttaagattgtagttatttttatgataagttaacttgtggcaagattggaattataagttttataaagacaataaatttgatcatattgtaacaattcctgtacctttagatgaactggtggttattatcaaatgaataatagtaagacttagtttatctcgatatgttaatagtggtagggttgtaaaaattagttaggtatggtaatatattttgaaattagtccgaaactattggataatactgttcctctaattaataaagatctgaaaaaggtgcactgttataattattgattacggtgtattttatatagttgtttaattctaaacattgtgtagcaacaatagtgattatgtacatgtaaaagactattgattctttgtaataagctgaaagtttatagtatttctggatcaaggttaggtaatcaagtgagttaaaattttccttgaaaagctttgaaaaaagactagagtaaacatatagttgaattataccaatgaagggtagtcatgataaatgtatcatttttgggtttaatgtctggaatatctgtattattaaagaccacggtaatagttgtaggtatttttgaaggaagtaatcatttggaggataatggtgctttagaaggttataatgataaaatcatggaaatagtaaatggttatcaatcttttgatggtaagtttgctgaggtattacctattattgggttagagaaagattttcctgttgtggtagttattggactgggtaaatctgaggattttgatgaaaataaagctttaaaagttggtggtgtaatatattctgaacttaatagaatgaaggtaccagatgcatcaattgttattaatactgatagtaatgtaagtgccaatattggttatggagcacttttacgtagttttaaatttgataaatatttcgtagagaaaaaagataaaaattcagtttatttgaataagttgcttctattttcaaagagtgatccacaagaggttactgctttgtttaatgatttaaaagctgaaggtgagtcaatattcttagctcgttcttttgtttcagagcctccgaatattttatatccagaaacgtatgctcagatgatatatgaagaattaagtaaggttggtgtaacagttgaagtctttgatgaagattacatgaaagcaaatcaaatgatggcacttcttggagtaggtcagggtagtgctaaaaaatctcgacttgtagttatgaaatggaatggaggtgatgagtcagaaagtcctattgcgtttgttggaaaaggtgtaacttttgatactggtggaatatccttaaaaccttcaaagggtatgtgggatatgaaatatgatatggcaggttctgcttctgttgttggaattatgcgtactcttgctgcaaggaaggcaaaagttaatgctgttggagtggttggattagttgaaaattcagtagatggaaatgcgcaaagacctagtgatgttgttatttcaatgtctggacaaacaattgaggtgttaaatactgatgcagagggtaggttggttttagctgatgctttatggtatactcaggagatgtttactcctaaattaatggtggatttagcaacattaactggtgcagtagtggttgctttaggtaataatcagtatgctgggcttttttcaaatgatgattctattgcaaatcagttgattgtagctgggaatgaatctggtgagaaattatggcggttacctttagatgaagcctatgataaacttatagattcatcaattgctgatatgcagaatatttcaacaaaaggatatggggcggatagtattactgcagcacagttcttacaaagatttgttaatggtgttccttgggtgcatttggatattgctggtatggcatgggattatgaaggcactgagatatgtcctaagggtgcaactggttttggggtaaggctattaaatagatttgtatcaaagtactatgagtctcattagttgtttcttctttatttatagtttaagtaaagtaatgtatcttgatttctatgttactcaatttaagtttatatagtaggctgattttttggtattgtgattgatttatgagtgtgctattgtggtataaatttatatttgtagttatatttatataggttcagttaatgactatgaaaccacttaggttaggtattttaatttcaggtaggggttctaatatgcaggctctgattaatgcttgtcagcgagatgattttcctgcaagtgtatcctgtgttatatcaaataaatcaaatgcaaacggtctaatacttgctcagcaaagtaatattaaaacttttatagtacaaggtcgtcctctagattttgatgctattgataatatacttgaagaacatgaggtggatttaatctgtcttgcaggatttatgagtattgttcctgaaaagtttattaataagtggttatataaggttattaatatacatccttctctcttgccatcatttaagggtttaaatgcacaagctcaagcattaaaggctggagtaaagattgctggatgtacagttcattatgtatacccagaagttgatggtggacctattattgttcaggcagcagttccagtgttttcatctgatagtgttgaggatcttgctaatagaatattgaagatggaacatatttgttaccctaaagctgtggaactaattgcttataatcagctacaacttaacggtagtttagctttatcagcaaaaacactacacatgttttataatgatgaagcttttgtatagcttatttttttatgtattgagccttatttttggataagttggtaaaacaatctacttttcctcgttataatactgtaaacaactatttctttgcctttgtttggtgttattaatgttagttatgtagtaagtaatgtttagttgtagcattgtattctaaaacattatttcttcttattatttaagagtgtattacttgtatagtattgataagatattcattatgaaaatatatattctacataatgagagttagtaaaatcatttataatgtaactattaaggtttatggtaatatcattaaatgttatgataggcctaggcttatcatatttgtattatcaagtgatgatagttttatattttaataaattttgtattggtttattataacgctataccatgaatgttatttgaagttatatggagtggaagattgaatccttacctgtaccttatgataaagctatgtgttttatgcaacaaagggtcgagggtattgctaataagacacaagatgaactagtatggttacttgaacattttccgttatatacggctggtactagtgcaaggagtgaggaattactaaccgatagtttatttcctgtatattctacaggtagaggtggtaaatacacttatcatggtcctggtcaaagaattgcttatgtgatgatggatttaaaagcaagagataaatgtaatgttaggttgtatgttgaaactttgggtgagtggattgttaaaactttaaagcatttttcaatacgatcn

SEQ ID NO: 39 - DNA, 1503 bp, Ehrlichia ruminantium (formerly Cowdria ruminantium)
  CDS (1)..(1503): Corresponds to SEQ ID NO38, nucleotides 816.. 2318 Hypothetical cytosol aminopeptidase Product = “13hworf1”
atgataaatgtatcatttttgggtttaatgtctggaatatctgtattattaaagaccacggtaatagttgtaggtatttttgaaggaagtaatcatttggaggataatggtgctttagaaggttataatgataaaatcatggaaatagtaaatggttatcaatcttttgatggtaagtttgctgaggtattacctattattgggttagagaaagattttcctgttgtggtagttattggactgggtaaatctgaggattttgatgaaaataaagctttaaaagttggtggtgtaatatattctgaacttaatagaatgaaggtaccagatgcatcaattgttattaatactgatagtaatgtaagtgccaatattggttatggagcacttttacgtagttttaaatttgataaatatttcgtagagaaaaaagataaaaattcagtttatttgaataagttgcttctattttcaaagagtgatccacaagaggttactgctttgtttaatgatttaaaagctgaaggtgagtcaatattcttagctcgttcttttgtttcagagcctccgaatattttatatccagaaacgtatgctcagatgatatatgaagaattaagtaaggttggtgtaacagttgaagtctttgatgaagattacatgaaagcaaatcaaatgatggcacttcttggagtaggtcagggtagtgctaaaaaatctcgacttgtagttatgaaatggaatggaggtgatgagtcagaaagtcctattgcgtttgttggaaaaggtgtaacttttgatactggtggaatatccttaaaaccttcaaagggtatgtgggatatgaaatatgatatggcaggttctgcttctgttgttggaattatgcgtactcttgctgcaaggaaggcaaaagttaatgctgttggagtggttggattagttgaaaattcagtagatggaaatgcgcaaagacctagtgatgttgttatttcaatgtctggacaaacaattgaggtgttaaatactgatgcagagggtaggttggttttagctgatgctttatggtatactcaggagatgtttactcctaaattaatggtggatttagcaacattaactggtgcagtagtggttgctttaggtaataatcagtatgctgggcttttttcaaatgatgattctattgcaaatcagttgattgtagctgggaatgaatctggtgagaaattatggcggttacctttagatgaagcctatgataaacttatagattcatcaattgctgatatgcagaatatttcaacaaaaggatatggggcggatagtattactgcagcacagttcttacaaagatttgttaatggtgttccttgggtgcatttggatattgctggtatggcatgggattatgaaggcactgagatatgtcctaagggtgcaactggttttggggtaaggctattaaatagatttgtatcaaagtactatgagtctcattag

SEQ ID NO: 40 - protein, 500 aa, Ehrlichia ruminantium (formerly Cowdria ruminantium)
MINVSFLGLMSGISVLLKTTVIVVGIFEGSNHLEDNGALEGYNDKIMEIVNGYQSFDGKFAEVLPIIGLEKDFPVVVVIGLGKSEDFDENKALKVGGVIYSELNRMKVPDASIVINTDSNVSANIGYGALLRSFKFDKYFVEKKDKNSVYLNKLLLFSKSDPQEVTALFNDLKAEGESIFLARSFVSEPPNILYPETYAQMIYEELSKVGVTVEVFDEDYMKANQMMALLGVGQGSAKKSRLVVMKWNGGDESESPIAFVGKGVTFDTGGISLKPSKGMWDMKYDMAGSASVVGIMRTLAARKAKVNAVGVVGLVENSVDGNAQRPSDVVISMSGQTIEVLNTDAEGRLVLADALWYTQEMFTPKLMVDLATLTGAVVVALGNNQYAGLFSNDDSIANQLIVAGNESGEKLWRLPLDEAYDKLIDSSIADMQNISTKGYGADSITAAQFLQRFVNGVPWVHLDIAGMAWDYEGTEICPKGATGFGVRLLNRFVSKYYESH

SEQ ID NO: 41 - DNA, 639 bp, Ehrlichia ruminantium (formerly Cowdria ruminantium)
  CDS (1)..(639): Corresponds to SEQ ID NO38, nucleotides 2486.. 3124 Hypothetical phosphoribosylamine-glycine ligase Product = “13hworf2”
atgactatgaaaccacttaggttaggtattttaatttcaggtaggggttctaatatgcaggctctgattaatgcttgtcagcgagatgattttcctgcaagtgtatcctgtgttatatcaaataaatcaaatgcaaacggtctaatacttgctcagcaaagtaatattaaaacttttatagtacaaggtcgtcctctagattttgatgctattgataatatacttgaagaacatgaggtggatttaatctgtcttgcaggatttatgagtattgttcctgaaaagtttattaataagtggttatataaggttattaatatacatccttctctcttgccatcatttaagggtttaaatgcacaagctcaagcattaaaggctggagtaaagattgctggatgtacagttcattatgtatacccagaagttgatggtggacctattattgttcaggcagcagttccagtgttttcatctgatagtgttgaggatcttgctaatagaatattgaagatggaacatatttgttaccctaaagctgtggaactaattgcttataatcagctacaacttaacggtagtttagctttatcagcaaaaacactacacatgttttataatgatgaagcttttgtatag

SEQ ID NO: 42 - protein, 212 aa, Ehrlichia ruminantium (formerly Cowdria ruminantium)
MTMKPLRLGILISGRGSNMQALINACQRDDFPASVSCVISNKSNANGLILAQQSNIKTFIVQGRPLDFDAIDNILEEHEVDLICLAGFMSIVPEKFINKWLYKVINIHPSLLPSFKGLNAQAQALKAGVKIAGCTVHYVYPEVDGGPIIVQAAVPVFSSDSVEDLANRILKMEHICYPKAVELIAYNQLQLNGSLALSAKTLHMFYNDEAFV

SEQ ID NO: 43 - DNA, 354 bp, Ehrlichia ruminantium (formerly Cowdria ruminantium)
  CDS (1)..(351): Corresponds to SEQ ID NO38, nucleotides 3548.. >3900 Hypothetical lipoate-protein ligase B Product = “13hworf3i”
atggagtggaagattgaatccttacctgtaccttatgataaagctatgtgttttatgcaacaaagggtcgagggtattgctaataagacacaagatgaactagtatggttacttgaacattttccgttatatacggctggtactagtgcaaggagtgaggaattactaaccgatagtttatttcctgtatattctacaggtagaggtggtaaatacacttatcatggtcctggtcaaagaattgcttatgtgatgatggatttaaaagcaagagataaatgtaatgttaggttgtatgttgaaactttgggtgagtggattgttaaaactttaaagcatttttcaatacgatcn

SEQ ID NO: 44 - protein, 117 aa, Ehrlichia ruminantium (formerly Cowdria ruminantium)
  misc_feature (354)..(354): n = a, c, g, or t
MEWKIESLPVPYDKAMCFMQQRVEGIANKTQDELVWLLEHFPLYTAGTSARSEELLTDSLFPVYSTGRGGKYTYHGPGQRIAYVMMDLKARDKCNVRLYVETLGEWIVKTLKHFSIR

SEQ ID NO: 45 - DNA, 4369 bp, Ehrlichia ruminantium (formerly Cowdria ruminantium)
gatcctctgattgaggaagatcaacaccacaatgtttttctttagtgtgatacataacatgatcaggatagaaactaggcactaacttactagttgcctcttcgtatataggattaccatttctgtcaaaatctaacattaaagaacaagtttcacttaaaccacgagtatttcttaaactatcttcaacagtattaaccaaattcaaacttgaaactaattggaacgtactttcttttccaacacatataacaggcttactatccttaacagtagtttcacaactagaagcagataaaactgaatcctctattttacctaaatctacacctgatagttttactttatcatcttttacattaccactttgaaaattaaaagtagcaccaacacttctagcaacactactgtccttttcatctcttatagcagtaccttcagatactacttttttctctacagatttatccttagtactacctgtacactctgtatcagtaacatcccttttctcatctggtttctctaatctagaaccagataatctaacttgattacttttcatatcaccagtttgagcactcgcacttacaaaaacatctctctttttttcatttattcttatagtatcaccttcaggtactacttttttctctacagatttatccttagtatcaaccataacatcagatttaaaactaagacttgtttttatagtagtttcagtatgactatagtgaacaccaacaccagcatttttcttaaccataacttgttgaccttctacactatcgcgttctacagtttgaacgtcagaaatttgatgtgttacctgatccattttggtagatgagacagtagattttgattttaaaacatgacctatatcatcactatcattatggtaaactgattttagaaattcaataccatcttttccagataaagtgtccatttcgtacttttcatattcactctgactacaataagtaaccctactttttgtatcattatctgatatatctaattctcttgacttatacttcataacactcaacacataacatacaaacctaaccacaagcagaacatagtaaacacatataaaaaacagcgacactgctgtgtataggattatctcattcatattatttaataagaaaattaatataagttaatatatacatatatatttaacaactaaaagaattataataaaaaaatttactatttctaatatttttttaattagttactaaatctatattatatttttaaataatactaatttttaatataaataaactaatatcaaatgataatcttttactatattagcaacatcataaagtgctaaatactacttcttatagaggatgatctactatcactcattacacaactcacattaatataagatggtaacctttctgaaggagtatatattttattcttcttaacccgttttacaacattacttataataatagcatctgaaatttctcccatattgttttttaaatattctttaactacaacagcagcttttttctccatcttatcactaaaaaatgaagccaatttctgaatagatttcaatatatcaagttcccatttatcattatttatcaccttagacattaatgtaccaaacactgaactttgagaatgatttccatgttctagatatctggttaccatattatgtacagccctatatttaagtcgcattttactaaccgccatgatcaaaacattcttatatgctactgtgatcgcttctttttgaatactctcatatacttgtgtgacatattcagaatacatataagattttacttgattctctaattcacagatcaaaaactctcgatcaccgggattagcatcacatactctcatatcctttgccaataagtaaacacattcttttataagtcctgctttagaccttttatacaatgttgtaccacgctgaatagcaaagtcccgtatcataacaggcattacttgtttatccatacgttgtatagtattcaacatcagactatatgcatccatacattcatgcttaactttactacgtaaattctcatcacctattcccgacaacagagtatctagtctatcactctcaatattccatgctgatatcaataaatcatgaggatcaatttgcattcctccttgtgctttgtggtaaaccattatttgagatatgtcacattcatcatctctaaacaagtatgtagtagtacttaaaccttgcttcttctttaacttatctgtattcaaacaagtagactcttgacctataactctaccttcactttcaatactatgttttacagatggtaatacagtacaatctacactactttcattttttctactttcacgaacacgattagtatcatgaataccacctattttactagaagccacactaattttctggccatgactaattgctggagttactaatgacaatctagcacgacttataggcgtagcctcttttctcctactacgctcatcagcatctttaacatgcctagcatcactcttcttaccagtaactgacctaactgcttgatcacaaacaaccaatctcttactattagcatgtacttttttatctcttgcatttatcattgctacagatctgttaccactctcaactatagcacgccccgcatacattatagcttctcgaaaaacttgcggattactaaaaatcaaggacatactagtcattggatcattaaacatcaaatacatattaattcgtggattactaaacattaagcttacattcagacacacatttgacaatagtttgaatagattttcacagttcatacttgtcatctgacttaacaattgttgatcaccagcattaccacttatcatactcctgattacaagtcttaaatctcgtgaagatatagcagtctggttattatcagaaacagcagtacttgtactactaacctttttagaatatgaaaattgtgcacaagaatctgatggtttaccagttgtatcatcttgaccaactaggaaactatgttcagatgatataatcccttccaaacctttatatgatgtatctgaaatagtatctcgcatattaataccacgaatagcaaggtcactcatatcacaaccagtagacttagtatcttcatgcttagataaatttctcaacaagccagtatcagcacttgcaaacacaaaatcgttctgtttatcaggccgaacaataaagtcattcccacctaaactatggttaagttttccaagactgccatgaaatccaacttctatcctatattgttgagagaaagtagatctatccaataatccaccaacaccaataaaactatcactagcatactgcccaactcttggattatcacccatacaaaaactatccggaaaaacactacgctttggagacaaataactattgaaatgttcaacttctgataataagttagttgtggaataagagcgctttttttgaacaccaccttcactatcatactgatttaatattctataaggtctatcccatctctgtataccatcactaaaaggtttaccccattcttctatatcgccactaaaaaatttatcccatttttctataccaccactaaaaggtttactccatctttcgagactattaccagtaagaacaccatcttcttgtaatcttttatcactttgtccgttatatagaaattgatcaccacatttagattctacatcctccatctccatgtacgtcatatcatctacaccctcaggggaactttcactatcttgatcacttgaattaacattttcatcatcagaagaacccactacaccagcacaatcttttcgttcttgtgattcctcactaggtatagttttagaacttactgaagaagcctcagattccccatccagattactatttgttaaagtatttcttccttctcttccatagattttcttacaacaatacactatacaactagcaattacgagcataataatgaacaccgtaaatacaataagcattagcattcttatattcatatttaatacttctttatagattatcattaataatatataattttttaatataaacaagactatttaataaaaatatactattttaacggagatttttatgataattcttaaaattataaatatatatcataacatgtaacaagttattgatataaaaaataaaataatattaaccttactaagttatattctaaaataattaaaaataatcttaaaatctattaataagtacttatatacaattatataataccattactaaataaccatacagatc

SEQ ID NO: 46 - DNA, 1147 bp, Ehrlichia ruminantium (formerly Cowdria ruminantium)
  misc_feature (1)..(1147): Complement to SEQ ID NO45, nucleotides <1.. 1147 Product = “14hworfli”
gatcctctgattgaggaagatcaacaccacaatgtttttctttagtgtgatacataacatgatcaggatagaaactaggcactaacttactagttgcctcttcgtatataggattaccatttctgtcaaaatctaacattaaagaacaagtttcacttaaaccacgagtatttcttaaactatcttcaacagtattaaccaaattcaaacttgaaactaattggaacgtactttcttttccaacacatataacaggcttactatccttaacagtagtttcacaactagaagcagataaaactgaatcctctattttacctaaatctacacctgatagttttactttatcatcttttacattaccactttgaaaattaaaagtagcaccaacacttctagcaacactactgtccttttcatctcttatagcagtaccttcagatactacttttttctctacagatttatccttagtactacctgtacactctgtatcagtaacatcccttttctcatctggtttctctaatctagaaccagataatctaacttgattacttttcatatcaccagtttgagcactcgcacttacaaaaacatctctctttttttcatttattcttatagtatcaccttcaggtactacttttttctctacagatttatccttagtatcaaccataacatcagatttaaaactaagacttgtttttatagtagtttcagtatgactatagtgaacaccaacaccagcatttttcttaaccataacttgttgaccttctacactatcgcgttctacagtttgaacgtcagaaatttgatgtgttacctgatccattttggtagatgagacagtagattttgattttaaaacatgacctatatcatcactatcattatggtaaactgattttagaaattcaataccatcttttccagataaagtgtccatttcgtacttttcatattcactctgactacaataagtaaccctactttttgtatcattatctgatatatctaattctcttgacttatacttcataacactcaacacataacatacaaacctaaccacaagcagaacatagtaaacacatataaaaaacagcgacactgctgtgtataggattatctcattcat

SEQ ID NO: 47 - DNA, 2766 bp, Ehrlichia ruminantium (formerly Cowdria ruminantium)
  misc_feature (1)..(2766): Complement to SEQ ID NO45, nucleotides 1354.. 4119 Hypothetical lipoprotein Product = “14hworf2”
ctaaatactacttcttatagaggatgatctactatcactcattacacaactcacattaatataagatggtaacctttctgaaggagtatatattttattcttcttaacccgttttacaacattacttataataatagcatctgaaatttctcccatattgttttttaaatattctttaactacaacagcagcttttttctccatcttatcactaaaaaatgaagccaatttctgaatagatttcaatatatcaagttcccatttatcattatttatcaccttagacattaatgtaccaaacactgaactttgagaatgatttccatgttctagatatctggttaccatattatgtacagccctatatttaagtcgcattttactaaccgccatgatcaaaacattcttatatgctactgtgatcgcttctttttgaatactctcatatacttgtgtgacatattcagaatacatataagattttacttgattctctaattcacagatcaaaaactctcgatcaccgggattagcatcacatactctcatatcctttgccaataagtaaacacattcttttataagtcctgctttagaccttttatacaatgttgtaccacgctgaatagcaaagtcccgtatcataacaggcattacttgtttatccatacgttgtatagtattcaacatcagactatatgcatccatacattcatgcttaactttactacgtaaattctcatcacctattcccgacaacagagtatctagtctatcactctcaatattccatgctgatatcaataaatcatgaggatcaatttgcattcctccttgtgctttgtggtaaaccattatttgagatatgtcacattcatcatctctaaacaagtatgtagtagtacttaaaccttgcttcttctttaacttatctgtattcaaacaagtagactcttgacctataactctaccttcactttcaatactatgttttacagatggtaatacagtacaatctacactactttcattttttctactttcacgaacacgattagtatcatgaataccacctattttactagaagccacactaattttctggccatgactaattgctggagttactaatgacaatctagcacgacttataggcgtagcctcttttctcctactacgctcatcagcatctttaacatgcctagcatcactcttcttaccagtaactgacctaactgcttgatcacaaacaaccaatctcttactattagcatgtacttttttatctcttgcatttatcattgctacagatctgttaccactctcaactatagcacgccccgcatacattatagcttctcgaaaaacttgcggattactaaaaatcaaggacatactagtcattggatcattaaacatcaaatacatattaattcgtggattactaaacattaagcttacattcagacacacatttgacaatagtttgaatagattttcacagttcatacttgtcatctgacttaacaattgttgatcaccagcattaccacttatcatactcctgattacaagtcttaaatctcgtgaagatatagcagtctggttattatcagaaacagcagtacttgtactactaacctttttagaatatgaaaattgtgcacaagaatctgatggtttaccagttgtatcatcttgaccaactaggaaactatgttcagatgatataatcccttccaaacctttatatgatgtatctgaaatagtatctcgcatattaataccacgaatagcaaggtcactcatatcacaaccagtagacttagtatcttcatgcttagataaatttctcaacaagccagtatcagcacttgcaaacacaaaatcgttctgtttatcaggccgaacaataaagtcattcccacctaaactatggttaagttttccaagactgccatgaaatccaacttctatcctatattgttgagagaaagtagatctatccaataatccaccaacaccaataaaactatcactagcatactgcccaactcttggattatcacccatacaaaaactatccggaaaaacactacgctttggagacaaataactattgaaatgttcaacttctgataataagttagttgtggaataagagcgctttttttgaacaccaccttcactatcatactgatttaatattctataaggtctatcccatctctgtataccatcactaaaaggtttaccccattcttctatatcgccactaaaaaatttatcccatttttctataccaccactaaaaggtttactccatctttcgagactattaccagtaagaacaccatcttcttgtaatcttttatcactttgtccgttatatagaaattgatcaccacatttagattctacatcctccatctccatgtacgtcatatcatctacaccctcaggggaactttcactatcttgatcacttgaattaacattttcatcatcagaagaacccactacaccagcacaatcttttcgttcttgtgattcctcactaggtatagttttagaacttactgaagaagcctcagattccccatccagattactatttgttaaagtatttcttccttctcttccatagattttcttacaacaatacactatacaactagcaattacgagcataataatgaacaccgtaaatacaataagcattagcattcttatattcatatttaatacttctttatagattatcat

SEQ ID NO: 48 - DNA, 3500 bp, Ehrlichia ruminantium (formerly Cowdria ruminantium)
gatcaaaagaaattggaatatcttcaacataaaatagggactcatttgctaaattaactaaattttttgttctcttttttagttcattaattccatttagtagatagcctattttctctgtattgactttatatcctattgttttctctatgaatattatatcaaatttaatcttgtaaagtataaaaccttggttctttatttactgtaatgaaagtataattattcttaatttactctaaaatatacaataagaagttatatctatttttttatatttattacttaatgactcatatatctatgattgcattgtaattatattagtaaatctagtgtcatggatgatctgtatctcactactgatttagttactgttgttttttgtataaatgataataaacttattacaaattgttttatggcaaaatttcaataatatttgtttttcatatattgacttcagtataaatttatagtagctttagcactctatcttttaccaggtttatgatgataagaatctttcttttgttaggcttagtattattagtagcaagttttccactattaaataactggctatctaatcattctggtaagtctactacattggataaggatgcagttatatctatagttgaggaatatataaccaattatcctcagagggtaatagatttacttactacaggccaagcacaagcagaaagagcagagcttactgaaaatattaaaaaatataaatctgagcttgaagatattgcatacccatctgctggcaataaagacagtaaaattgcatttattgagttcttcgattactcttgtggttattgtaaaatgatgtttgaagatatcaaacaaattataaaagatggtaaggtacgtgttatttttagagattttccaatacttggggaatcgtcgttaaaggctgttaaagcagcattggctgtacatcttatcaatccaagtaaatacttggacttctattatgcagcattaaatcataaacagccatttaatgatgaatctatacttaatatagttaaatcacttgaaatttcagaagaggaatttaaagattctttatctaaaaattctagtactattgataagatgatagagtccactagaaatctggctgagaagttaaatatcagaggtactcctgctcttataataggtgatgcattcattgggggagctgcagatttatcaactttaagaagtaaaatagtagaacagcaggaacaataaagcttctttccttaatgaagaagggtgttatctgtgctatttatagaagggcttgtagggtcttcagttattgttattctgttaggaacaccttcttcgatatcatcatgttctgcattattgttttcatcagtaatggtgtaggttttactgcatatgattccataagctatcattgatagtgcaataatggcacttgctatagcaataatattaattattatatttttattattaacctcaaagtggtataacaatgatattgttataatactaaaaagtataattaatcctgctgtaaatgcacaacatagggtttttttgggatatggttgctgttgagttggtttttttgtatgactgctaatactatagctatcatcatatgtatcagttatgtgaccttcactttctttagtactagtgtgagggtagtgaattgctaaagcttgtgctgtagcgtatatattaggaatatataattgagccatagaaaatcccttagctaacaatgatacaggttgtatatattgatcaattgaaaattttaattgatcactcaatacattatcaccaacctttttaactacatcatgtgataggcaaatatttccatttttatcaactaaaggacaaactaacttttgtatatcgaaaggagagagcaattgtgatgttgtaattgatgacacagtatcccaagaaacacactcctgaagtccttcttcaaatgctttaagtagtggatattgtaggttcttagcactattacacaatagatctatatttcgtactataggaattgaaactagaatattaaataataaaaattgtatggaagatgtttgggctgtacataaatattgagaaaattgctcatcactgaaagaacttacataacatgcagctaattgtatggtacgactaaacaaagaaatgtcaaagttttggcatgatatttcccttgtttccgtagtacgtaccaactgcatacgtaatacaacctcccttattgttgtattgcagaactcagtactgtacagtgtatcttgtgttgttactatatgatctgtatcgacacctaattcctctgaaaaagaatcaaggacattatgtattgtcatatagttgtgaaaaggttcagcattggcttttgcaaaatcatcagctgatgcataatggtataggttatttacatcgctgtttagtagatttgctagtaaagaaagtgttatataaaacttattaatacctgatgttacatgtgataataaggtttcagggttgttataagcaacatatgcaatatcaaataatcttgtaatgcaagatttttctaaaaaattaccaaaacatagctttaataaaattattagtacactgctagaatagtgttccggatcaagagaatgttgccctaatactctacacattataactgagatttgtcgagcttttctttcattaacagtactacaacaagctatcatgtgatatggtaattgtgtattgcctaatatgtatggtacaccttcattaaatgttaggtgttgcaataacatgaccatgttatttggtataatctgagtatttttattatatatattagctgtgctacaatgtaactttgcttgaatttcattaaaaagtacttcatacccccgaagtaatttgatctgttttttatgtggggcatttgcaatcttcattaagagagcataacatgtattataatattcttgtatttttgaagctggtaattttagatggaatgcggaataataacaaagggctagtaactctaacaaatgtcctgataattgtttgtgagctccatgtaattctgctaatatgtctctatagtctttactttgcgaaataaacttttgtaatagtacttccttatatattgcacttatgacatgtaatgtatttaactttatggcataagtagtattaagtgtactgcgtattgcacatgagatatcgcaactaataaaactagtgttattgcgtgggataacttgaaatgtacttgttgtttgtaagttttcaaaggttctcattgtacagttaatgtcttgagaaagagatgctgacattaagtactttggactgcagatagagagacaatcctggtacattgcatagaaaatatccttataggcattaagtattaatctagttaacttgccaacatttttgttttcgtatactgtattagataaagatagatc

SEQ ID NO: 49 - DNA, 753 bp, Ehrlichia ruminantium (formerly Cowdria ruminantium)
  CDS (1)..(753): Corresponds to SEQ ID NO48, nucleotides 511.. 1263 Hypothetical outer membrane protein Product = “18hworf1”
atgatgataagaatctttcttttgttaggcttagtattattagtagcaagttttccactattaaataactggctatctaatcattctggtaagtctactacattggataaggatgcagttatatctatagttgaggaatatataaccaattatcctcagagggtaatagatttacttactacaggccaagcacaagcagaaagagcagagcttactgaaaatattaaaaaatataaatctgagcttgaagatattgcatacccatctgctggcaataaagacagtaaaattgcatttattgagttcttcgattactcttgtggttattgtaaaatgatgtttgaagatatcaaacaaattataaaagatggtaaggtacgtgttatttttagagattttccaatacttggggaatcgtcgttaaaggctgttaaagcagcattggctgtacatcttatcaatccaagtaaatacttggacttctattatgcagcattaaatcataaacagccatttaatgatgaatctatacttaatatagttaaatcacttgaaatttcagaagaggaatttaaagattctttatctaaaaattctagtactattgataagatgatagagtccactagaaatctggctgagaagttaaatatcagaggtactcctgctcttataataggtgatgcattcattgggggagctgcagatttatcaactttaagaagtaaaatagtagaacagcaggaacaataa

SEQ ID NO: 50 - protein, 250 aa, Ehrlichia ruminantium (formerly Cowdria ruminantium)
MMIRIFLLLGLVLLVASFPLLNNWLSNHSGKSTTLDKDAVISIVEEYITNYPQRVIDLLTTGQAQAERAELTENIKKYKSELEDIAYPSAGNKDSKIAFIEFFDYSCGYCKMMFEDIKQIIKDGKVRVIFRDFPILGESSLKAVKAALAVHLINPSKYLDFYYAALNHKQPFNDESILNIVKSLEISEEEFKDSLSKNSSTIDKMIESTRNLAEKLNIRGTPALIIGDAFIGGAADLSTLRSKIVEQQEQ

SEQ ID NO: 51 - DNA, 2226 bp, Ehrlichia ruminantium (formerly Cowdria ruminantium)
  misc_feature (1)..(2226): Complement to SEQ ID NO48, nucleotides 1275.. >3500 Product = “18hworf2i”
ttaatgaagaagggtgttatctgtgctatttatagaagggcttgtagggtcttcagttattgttattctgttaggaacaccttcttcgatatcatcatgttctgcattattgttttcatcagtaatggtgtaggttttactgcatatgattccataagctatcattgatagtgcaataatggcacttgctatagcaataatattaattattatatttttattattaacctcaaagtggtataacaatgatattgttataatactaaaaagtataattaatcctgctgtaaatgcacaacatagggtttttttgggatatggttgctgttgagttggtttttttgtatgactgctaatactatagctatcatcatatgtatcagttatgtgaccttcactttctttagtactagtgtgagggtagtgaattgctaaagcttgtgctgtagcgtatatattaggaatatataattgagccatagaaaatcccttagctaacaatgatacaggttgtatatattgatcaattgaaaattttaattgatcactcaatacattatcaccaacctttttaactacatcatgtgataggcaaatatttccatttttatcaactaaaggacaaactaacttttgtatatcgaaaggagagagcaattgtgatgttgtaattgatgacacagtatcccaagaaacacactcctgaagtccttcttcaaatgctttaagtagtggatattgtaggttcttagcactattacacaatagatctatatttcgtactataggaattgaaactagaatattaaataataaaaattgtatggaagatgtttgggctgtacataaatattgagaaaattgctcatcactgaaagaacttacataacatgcagctaattgtatggtacgactaaacaaagaaatgtcaaagttttggcatgatatttcccttgtttccgtagtacgtaccaactgcatacgtaatacaacctcccttattgttgtattgcagaactcagtactgtacagtgtatcttgtgttgttactatatgatctgtatcgacacctaattcctctgaaaaagaatcaaggacattatgtattgtcatatagttgtgaaaaggttcagcattggcttttgcaaaatcatcagctgatgcataatggtataggttatttacatcgctgtttagtagatttgctagtaaagaaagtgttatataaaacttattaatacctgatgttacatgtgataataaggtttcagggttgttataagcaacatatgcaatatcaaataatcttgtaatgcaagatttttctaaaaaattaccaaaacatagctttaataaaattattagtacactgctagaatagtgttccggatcaagagaatgttgccctaatactctacacattataactgagatttgtcgagcttttctttcattaacagtactacaacaagctatcatgtgatatggtaattgtgtattgcctaatatgtatggtacaccttcattaaatgttaggtgttgcaataacatgaccatgttatttggtataatctgagtatttttattatatatattagctgtgctacaatgtaactttgcttgaatttcattaaaaagtacttcatacccccgaagtaatttgatctgttttttatgtggggcatttgcaatcttcattaagagagcataacatgtattataatattcttgtatttttgaagctggtaattttagatggaatgcggaataataacaaagggctagtaactctaacaaatgtcctgataattgtttgtgagctccatgtaattctgctaatatgtctctatagtctttactttgcgaaataaacttttgtaatagtacttccttatatattgcacttatgacatgtaatgtatttaactttatggcataagtagtattaagtgtactgcgtattgcacatgagatatcgcaactaataaaactagtgttattgcgtgggataacttgaaatgtacttgttgtttgtaagttttcaaaggttctcattgtacagttaatgtcttgagaaagagatgctgacattaagtactttggactgcagatagagagacaatcctggtacattgcatagaaaatatccttataggcattaagtattaatctagttaacttgccaacatttttgttttcgtatactgtattagataaagatagatc

SEQ ID NO: 52 - DNA, 4750 bp, Ehrlichia ruminantium (formerly Cowdria ruminantium)
gatcctgctaatactttaggtaatgtttttttaagatgtggcctagataaataaaacactgcttgactgtctatatagtacatacgtgatagataacgaaaaaaatttctcaaagcagacagtgaccttgcattggtaactgcttcaactccacattgatatctagaagcaaaccattttctcaaatctccaattttaacatgcacaagattatttaatgtaacagaacaccctgtactcttatgcaagaactctataaacttatcaaggtctcttacataagaaacaactgtattcaaagaatacctcttctcttgttccatccatattttccaattatttacaatatcatagagttctttattcgacatacatattaaaatcaatcaaaacaaaataatagtactatctaattaataaaaaaatcctcaatagtctttagcaagaactacaaatataatgagacacttaacttattaatatatttcaaatttattatactactcatcatatttagcataatatcacatctgacatgattaacaatagtaacacatttcttaacactagataatatacaataaataatatataatttttgactggtttcttgatataaattaggtaaaaaatgaaaatcagtattttaggtgcaggatcatttggcacagcaatagcaattgcactgtcagcacatggtatatcagttaacttatggggacgtgatcatagaaatattacacatataaacacttaccgaaaaaatttaaaatatttacccacatatcatctaccagacaacatatatgcaaccagcaatatagacgaagtattatctgacaacaatacatgtattatcttaactattcctacacaacaattacgcaccatatgtacacaaatacaacacaaacagcatatgtgtaaaaatactccaatattaatttgtagtaaaggtatcgaaattacatcactcaaatttcccagtgaaatagcagaagaaattttacaatataatccaatttttatactctctggtccaagttttgctaaagaaattgcagaacatcttccttgtagtatagtacttgctggtgataataaagaacttggtgaatcattgatagaaacaataagtaatgatgttctaaaaataatataccatcaagatattataggtgtacagattggagctgcattaaagaacataattgcaattgcatgtggaataatcgctggaaaaaatttaggtaataatgctgttgctactgttataactaaaggcatgaatgaaattaaaacactatatatagcaaaaaatcattcaatagatcttcatacattaattggtccatcatgtcttggagatctaatattaacatgtacaacagaacattcacgcaatatggcttttggactagaaataggaaaaggtagaaatataaatacattaatagatcacaacctaaagcttgttgaaggaaccagtactgtaaaaccactgatatcattagcaaaaaaacttaatgtagaactaccaatttgcatatctatttacaatttattacatgagaatatatcactagataaagccatatcaaacatattatcttagtctatcatcctttgataatttcaacacaatgataaataaacggtcaaaaatatagaatacaacgttcaccacatatataaaaagcagtcaaatacccatcgttaacatccaactaagtatgtaacatattacataacaaacttaagtataacttaaaatttcctagctaattatataaacaaattcctatctcctatctcaattaaccaattgttatcagattataaataagtcaaattaaacttttacttatttataaaataatatgtttgcacactttttcactacatcatgtatataatgaacataattttacataaaataactaataaaaagaaccaatacatatattaaattactaaaacacctatttactacgtaatattacatagatgtcctgacataataaattctatcatccttgacatatttcaaacataaaatatgataaactgtttttttatgcccattagttcttaataaaattgctaattacccaaaaaggttaaaatacacattcctgcaacaatatgaagaagatacaatacacgtcaaataaattaaacaaatttctaatataaccacctactctaaacataggaaccaatagtatatcatacctaagtaagatatccctacagcatgtatcaaataataaagaaaaatcgttatgtatatttatacaattacaaaatttccaacttcaacaacaaaactcaaagttctattataacaaattcaagaattatatatacttcaccaacacatagttatcaaattctataatacatcatatcctttttaaaaagatgacttcacaattcatacataaataattatacatgcaatgtttattcagaaacattacacaatccaagaattttcaatttacggtgtaatgctgaacgttccattccaacaaattcagcagttcgtgatacattacctccaaaacgagataactgagtttttaaatactgtctttcaaattcttcacgagctttacgtaatggtacagaaataactttagcacttaaaacatcattaataggcgaattagatactatatctactggtaaatcttttgctgtaatcatctccttaggagatttcataattaaaatccattctataacattacgtaattgtcttaagttacctggccattcatatgactgcattgctattaaagcttcatcacttaatacatgagtacacaaacctatttttttacagatgctattcataaaatacctacacaattccggtatatctgtacaatattctactaaagacggtactctaattggaaggacatttaatctataatataaatcctcacaaaacctaccagcttttacttcactttcaatatctttggaagaagacacaataattctcacatctatactaacaggaatcttactattttccctatatatttttccctcctgtaataatctgagtaatcttaattgcgtatcgtatcgtaaatctgttacttcatctataaataacgtaccatgatttgcttgctctataattccaatatgaggaggtactctatgagacaatatattattactttcctcactaccaaatatattaaccaagtaattattagctggtagcatagatgagtacatagatataaatggagtatcataccccttggattttttatgtattagcctagcaactacttcctttccaacacctggcgaaccagtaatgagtatacgactcgatgtagtagctgctttattaatcatacttctcaaattacgtataacaggggagttaccgactatttcataatcctcaaatgctgatttcaactcatcattttctctacgtaatctaccagactctatagctctctttacaactaactttaatcttccttctgtaaaaggcttttctatataatcataagcacccatatgcagagactttacagcagtggcaatattaccatgcccactaatcataataacaggcaaataaggatacctttctttaagcttttccagtacacttaatccatcaatatcagatcctcttaaccatatatccaataatacaacatcaggctctttttcataagccatcttgatcgcggataaaccatcaactgctaatttagtgacataattatcatcacttaatatatcttttattagatttctgatatcaacttcatcatcaacaactaatacttcagaaatatacaatctttccttggacatttcaaaatcctgtgccataattacctacaatactaatgaaatactgcaaacacaatgtactcctttaaagtacacctcataatactaccacacacactaatgatcaacaaccaatatattaaattcattattttttacttataaatttaaacatattataaaaattagttactattattactaatttatcaagcataggtttcctatatatccaaaaagaaaaataaaaacacttaataaaaaacttatcatcaactttgtaaacctatagtaactaattattaataaattatttcattatattaaccttccctggaacacgcatgtctatagttttccatactaaaaactcacttgaagctttatagatattcaataattcacgccaagcagcataagaatcattattaggtaattttatatttaaaccactagacaaaataatatcccaccaatgactatcaacatatgtgatagatgaaaccataccaccaacaagagtattatcattgactacttcacgtataaaatctagatgagttaacgcaccatctccatgtattgaagtcaaatcatcccgtatattacaattatcaacaataatatgtccaaaactatcaatgatagaatttttgttatcatgataccaatttgcaaaagcagaatattcttgaactgttatctgtaaagtatttggtaacagcctcttaactgaggcatttttgatccatggatgactagactctattttgtttctcaaatcagccaaaggcacaaaaaagatagaccttgcatcaacaagtttacgtatttcatcagaacttacatattcattaccatcaatcaatattttatcaacagtaaatccacaattgactaattgatc

SEQ ID NO: 53 - DNA, 371 bp, Ehrlichia ruminantium (formerly Cowdria ruminantium)
  misc_feature (1)..(371): Complement to SEQ ID NO52, nucleotides <1..371 Hypothetical integrase/recombinase Product = “19hworfli”
gatcctgctaatactttaggtaatgtttttttaagatgtggcctagataaataaaacactgcttgactgtctatatagtacatacgtgatagataacgaaaaaaatttctcaaagcagacagtgaccttgcattggtaactgcttcaactccacattgatatctagaagcaaaccattttctcaaatctccaattttaacatgcacaagattatttaatgtaacagaacaccctgtactcttatgcaagaactctataaacttatcaaggtctcttacataagaaacaactgtattcaaagaatacctcttctcttgttccatccatattttccaattatttacaatatcatagagttctttattcgacat

SEQ ID NO: 54 - DNA, 984 bp, Ehrlichia ruminantium (formerly Cowdria ruminantium)
  CDS (1)..(984): Corresponds to SEQ ID NO52, nucleotides 632.. 1615 Hypothetical glycerol 3-phosphate dehydrogenase Product = “19hworf2”
atgaaaatcagtattttaggtgcaggatcatttggcacagcaatagcaattgcactgtcagcacatggtatatcagttaacttatggggacgtgatcatagaaatattacacatataaacacttaccgaaaaaatttaaaatatttacccacatatcatctaccagacaacatatatgcaaccagcaatatagacgaagtattatctgacaacaatacatgtattatcttaactattcctacacaacaattacgcaccatatgtacacaaatacaacacaaacagcatatgtgtaaaaatactccaatattaatttgtagtaaaggtatcgaaattacatcactcaaatttcccagtgaaatagcagaagaaattttacaatataatccaatttttatactctctggtccaagttttgctaaagaaattgcagaacatcttccttgtagtatagtacttgctggtgataataaagaacttggtgaatcattgatagaaacaataagtaatgatgttctaaaaataatataccatcaagatattataggtgtacagattggagctgcattaaagaacataattgcaattgcatgtggaataatcgctggaaaaaatttaggtaataatgctgttgctactgttataactaaaggcatgaatgaaattaaaacactatatatagcaaaaaatcattcaatagatcttcatacattaattggtccatcatgtcttggagatctaatattaacatgtacaacagaacattcacgcaatatggcttttggactagaaataggaaaaggtagaaatataaatacattaatagatcacaacctaaagcttgttgaaggaaccagtactgtaaaaccactgatatcattagcaaaaaaacttaatgtagaactaccaatttgcatatctatttacaatttattacatgagaatatatcactagataaagccatatcaaacatattatcttag

SEQ ID NO: 55 - protein, 327 aa, Ehrlichia ruminantium (formerly Cowdria ruminantium)
MKISILGAGSFGTAIAIALSAHGISVNLWGRDHRNITHINTYRKNLKYLPTYHLPDNIYATSNIDEVLSDNNTCIILTIPTQQLRTICTQIQHKQHMCKNTPILICSKGIEITSLKFPSEIAEEILQYNPIFILSGPSFAKEIAEHLPCSIVLAGDNKELGESLIETISNDVLKIIYHQDIIGVQIGAALKNIIAIACGIIAGKNLGNNAVATVITKGMNEIKTLYIAKNHSIDLHTLIGPSCLGDLILTCTTEHSRNMAFGLEIGKGRNINTLIDHNLKLVEGTSTVKPLISLAKKLNVELPICISIYNLLHENISLDKAISNILS

SEQ ID NO: 56 - DNA, 1416 bp, Ehrlichia ruminantium (formerly Cowdria ruminantium)
  misc_feature (1)..(1416): Complement to SEQ ID NO52, nucleotides 2467.. 3882 Hypothetical nitrogen assimilation regulatory protein Product = “19hworf3”
ttattcagaaacattacacaatccaagaattttcaatttacggtgtaatgctgaacgttccattccaacaaattcagcagttcgtgatacattacctccaaaacgagataactgagtttttaaatactgtctttcaaattcttcacgagctttacgtaatggtacagaaataactttagcacttaaaacatcattaataggcgaattagatactatatctactggtaaatcttttgctgtaatcatctccttaggagatttcataattaaaatccattctataacattacgtaattgtcttaagttacctggccattcatatgactgcattgctattaaagcttcatcacttaatacatgagtacacaaacctatttttttacagatgctattcataaaatacctacacaattccggtatatctgtacaatattctactaaagacggtactctaattggaaggacatttaatctataatataaatcctcacaaaacctaccagcttttacttcactttcaatatctttggaagaagacacaataattctcacatctatactaacaggaatcttactattttccctatatatttttccctcctgtaataatctgagtaatcttaattgcgtatcgtatcgtaaatctgttacttcatctataaataacgtaccatgatttgcttgctctataattccaatatgaggaggtactctatgagacaatatattattactttcctcactaccaaatatattaaccaagtaattattagctggtagcatagatgagtacatagatataaatggagtatcataccccttggattttttatgtattagcctagcaactacttcctttccaacacctggcgaaccagtaatgagtatacgactcgatgtagtagctgctttattaatcatacttctcaaattacgtataacaggggagttaccgactatttcataatcctcaaatgctgatttcaactcatcattttctctacgtaatctaccagactctatagctctctttacaactaactttaatcttccttctgtaaaaggcttttctatataatcataagcacccatatgcagagactttacagcagtggcaatattaccatgcccactaatcataataacaggcaaataaggatacctttctttaagcttttccagtacacttaatccatcaatatcagatcctcttaaccatatatccaataatacaacatcaggctctttttcataagccatcttgatcgcggataaaccatcaactgctaatttagtgacataattatcatcacttaatatatcttttattagatttctgatatcaacttcatcatcaacaactaatacttcagaaatatacaatctttccttggacatttcaaaatcctgtgccat

SEQ ID NO: 57 - DNA, 597 bp, Ehrlichia ruminantium (formerly Cowdria ruminantium)
  misc_feature (1)..(597): Complement to SEQ ID NO52, nucleotides 4154.. >4750 Hypothetical cell division protein ftsQ Product = “19hworf4i”
ttatttcattatattaaccttccctggaacacgcatgtctatagttttccatactaaaaactcacttgaagctttatagatattcaataattcacgccaagcagcataagaatcattattaggtaattttatatttaaaccactagacaaaataatatcccaccaatgactatcaacatatgtgatagatgaaaccataccaccaacaagagtattatcattgactacttcacgtataaaatctagatgagttaacgcaccatctccatgtattgaagtcaaatcatcccgtatattacaattatcaacaataatatgtccaaaactatcaatgatagaatttttgttatcatgataccaatttgcaaaagcagaatattcttgaactgttatctgtaaagtatttggtaacagcctcttaactgaggcatttttgatccatggatgactagactctattttgtttctcaaatcagccaaaggcacaaaaaagatagaccttgcatcaacaagtttacgtatttcatcagaacttacatattcattaccatcaatcaatattttatcaacagtaaatccacaattgactaattgatc

SEQ ID NO: 58 - DNA, 4544 bp, Ehrlichia ruminantium (formerly Cowdria ruminantium)
gatccaattacacaaaagaaattgtctgatgctatcattaaagaagcaagagatttaaacttatataatgcaatcactgataatggagccggaggtctatcatcttctataggtgaaatgggaaataatggatttaaagtagaactaaataaagtattattaaaacataaaaacatgctaccttgggaaatttgggtatcagaatcacaagaaagaatgacattagctattcctccaagcaaattcccaatatttgaaaaaattatgaaaaagcatgatgttgaaatcagtattattggaacattcaataatacaaaaaaagcagtagtatcatataatgactccattattatggatatggatataaacttcttacataacggtataccaaaaactcatctaaaaaccataccatggtcaaacataatatcctcagtagtagacacattacataataaaccactagacactgagctaaatgaaatgatgcaaagaatgaatatatgtagtaaagaatttatctctacacaatatgatcatgaagtacagggaacatcagtcataaaacctatacaagggaaaggacgagtagatggagaagcaatagttattagaccaatactatcatcagaaaggggactagtaaaatcacatggactaggatcaagctatggagaaattagtacataccacatggctgcatgtgctatagatacagcaatacgtaattatatagcaatcgggggaaatttccatcacttagcattattagataatttctgttggtgtgactctacaaatccaaaaagattatggcaattaaaaaatgctgcccaagcatgttatgaatacgcaaaaattttcaaaacacctttcatttctggaaaagatagcatgttcaatgattttaaaggttataacaataaaggagaacctattaacatttctgctcctccttcacttttaatttctacagtaggaataatagaaaatattcacaatgccataacacttgatgtaaaaaatccaggagatttaatatacatattaggtgtgacatatgatgaacttggaaggtccgaataccaaaaatatagcggattaggaaataataatgttccacaagtacgtgctaaacatgcaaaaaaactatacaagttatatagcaatgcagttaatacaaatattatagcatctgcaattgcattaaacctaggggggctaattataggtttaataaaatcactaattggaggagaactgggagcaaaaattgacttatcactagtaccaacacataatattgaagataataacataaaagagaaagtaatcctattttcagaatcacaaagtagaattttagtaacaatagctccacataataaacaaaaatttgaaactatttttaaagacatagcacatgcaaacataggtataatcagtgatacaaataccctgattattaacaatatgcacattattaatttaaatacactaaaacacagttataaaaaattcagcaacatgaaaatacaagcatatgcagatgcagaatatatttagttagtaattaattttacttttattaccttaatagtcttagttgatatataaattttatatactatggatagatattatgcattattgaaaatatctataatgttaatccaaatgaactgaaacatagttataagaaattcaccaatatgaaaatacgagtaataaaactattgtttttattctactacattaataattgaaccttgatccaatcttggtatattacagcatcgtgattatcaaaaacatgtatacactaataaaattataaaaaactcaatattttaataatatatatgtatacataagaacaatatattttttacaactcaattattaagccatacagattatagtttcttgtttaataaataagtaaaaataatacaaaatctatttttctaatttatttaaaacgaatttatatactggacctactgtatactatataaggaatataaaaatacacttataatcagattagaaatttatcataatatttcaatatatttgttaatttcattatctaattgatatctatatataatgtcctctccttactaattataacaccttatttgaatactaaaatcatgtaatacttttctcaacttaaaaattgaaatgaccaaaaatctattctataattcatatcctttgtaataatttaatttaaactatagaaatggcttactcacctgacaatgatatagtagtactagcattaggtagtaattgtggcagtatgttattgaatattaaatctgctataaatatgttatctttatataacaaaacatattcttatatctataaaagtatggcactattaccagaaaattctagtagtgattgggatactccttttctgaatatggtagtatcaggttatacaaatctttcaccaaatcttatgttagaaagagttaaatacattgaaaaaaaaataggcaggtttaataatgaatactggtcacctagatgtatagatattgacattatcttatggggagataaagtcttagactcacaaactttatctattcctcataagcatatgcaagatagagattttgtacttgtaccactctgtgatattcacgcaagatttcctcatccagtatcaaagctatcaattgaagaaatagttctcaatctacatgagatcaatttaataaagcagtcatatattataactcaatatttatagatgacacttatagtattaatattgttatcattaagtacatatggtatatatatcaggaaatatttctcaatctacgtagtcaacctaataagtcaatcatatattatactcaatatttatagatgacacttatagcattaacagtgttatcattaagtacatataatggtatatatatcaggaaatacttctcaatctacgtaagtcaacctaataaagcagttatatattataactcagtatttatatataatatttagagtattaacagtactatcataagtatatgtatagtatagcaagttatattagattatatttaaaaatacataaccagatcataataacatgaatgaaaaacacaagatgatataaaaagttttttagttacaccatatcattggcaatccatatttattacttatcatattattcagaaattattactttccttcactgattcctgcaatatatataataaatttattttcacatcttatataaatttagaaaattccttgtaaatcaatatactagggttatagtgtcaatcgttaattaatactactccctacacaatgccgtataagtgcttttaaattatataaatactttaccccacaataacaaaataataacttttattttatacataaataagcataatcacataaatcaataaacacagtagtataccaaaactaaaattactctttactgtcaatatcaaacacattagcttataaattttcaccattccaaataacactagaacgtttcatttttataacctaactctacataaccaacatcctttaattaatatcaacattgatagatcactttttaataaatacaataaaattgtcggatattaatactataaataaaacttaatatctcttattttcataacttattctccctaaaacaatacgctataacacaaatatttaccctgatagattctaaatgcttataaacatgcgcctgattacactaacaaaatacagcgttcaatataatattagttacctaaactaacaaactaaagtagcatatttaaattagaaaattatttttaccataaattgctataatacatttataggttattatcctaatacaatagcaccttttatctatacttaactatctaatctaatatgccacagcagtactatttaacactaatttatagaagaaaattattttttatcacaaaatgctacacacttcctgtaagtggtaatttttccaccacaaccagtaaaacataaatcataatcactcctacataatctatcacaatcatttaccttacataaacttacaggtttcacaggaactacagattttataccagctccaaaagattttgaagtactactactttgtacttcatgcttaaaagatcctgaagaaaaactcatatcagttttaactcgttgctcagtatcacacacattaaattgcatatcacattcagtatcacaaaaagtaccatttttacatgatttcatacatttttgtctcttatcctcacatggattattactatcttctttatcatcattacctttactgatagtattgtttattatactaataaatggattacgacgagtttgtgcatcaatcattttctctgctaatcgacaatctgttaacttaacctgacattgcctatagcaatcattcttattgtaactacattgaagtatacatgctttgccaacttcattgtctggagcttcatagtttgatc

SEQ ID NO: 59 - DNA, 1566 bp, Ehrlichia ruminantium (formerly Cowdria ruminantium)
  CDS (1)..(1566): Corresponds to SEQ ID NO58, nucleotides <1.. 1566 Hypothetical phosphoribosylformylglycinamidine synthase Product = “21hworfli”
gatccaattacacaaaagaaattgtctgatgctatcattaaagaagcaagagatttaaacttatataatgcaatcactgataatggagccggaggtctatcatcttctataggtgaaatgggaaataatggatttaaagtagaactaaataaagtattattaaaacataaaaacatgctaccttgggaaatttgggtatcagaatcacaagaaagaatgacattagctattcctccaagcaaattcccaatatttgaaaaaattatgaaaaagcatgatgttgaaatcagtattattggaacattcaataatacaaaaaaagcagtagtatcatataatgactccattattatggatatggatataaacttcttacataacggtataccaaaaactcatctaaaaaccataccatggtcaaacataatatcctcagtagtagacacattacataataaaccactagacactgagctaaatgaaatgatgcaaagaatgaatatatgtagtaaagaatttatctctacacaatatgatcatgaagtacagggaacatcagtcataaaacctatacaagggaaaggacgagtagatggagaagcaatagttattagaccaatactatcatcagaaaggggactagtaaaatcacatggactaggatcaagctatggagaaattagtacataccacatggctgcatgtgctatagatacagcaatacgtaattatatagcaatcgggggaaatttccatcacttagcattattagataatttctgttggtgtgactctacaaatccaaaaagattatggcaattaaaaaatgctgcccaagcatgttatgaatacgcaaaaattttcaaaacacctttcatttctggaaaagatagcatgttcaatgattttaaaggttataacaataaaggagaacctattaacatttctgctcctccttcacttttaatttctacagtaggaataatagaaaatattcacaatgccataacacttgatgtaaaaaatccaggagatttaatatacatattaggtgtgacatatgatgaacttggaaggtccgaataccaaaaatatagcggattaggaaataataatgttccacaagtacgtgctaaacatgcaaaaaaactatacaagttatatagcaatgcagttaatacaaatattatagcatctgcaattgcattaaacctaggggggctaattataggtttaataaaatcactaattggaggagaactgggagcaaaaattgacttatcactagtaccaacacataatattgaagataataacataaaagagaaagtaatcctattttcagaatcacaaagtagaattttagtaacaatagctccacataataaacaaaaatttgaaactatttttaaagacatagcacatgcaaacataggtataatcagtgatacaaataccctgattattaacaatatgcacattattaatttaaatacactaaaacacagttataaaaaattcagcaacatgaaaatacaagcatatgcagatgcagaatatatttag

SEQ ID NO: 60 - protein, 521 aa, Ehrlichia ruminantium (formerly Cowdria ruminantium)
DPITQKKLSDAIIKEARDLNLYNAITDNGAGGLSSSIGEMGNNGFKVELNKVLLKHKNMLPWEIWVSESQERMTLAIPPSKFPIFEKIMKKHDVEISIIGTFNNTKKAVVSYNDSIIMDMDINFLHNGIPKTHLKTIPWSNIISSVVDTLHNKPLDTELNEMMQRMNICSKEFISTQYDHEVQGTSVIKPIQGKGRVDGEAIVIRPILSSERGLVKSHGLGSSYGEISTYHMAACAIDTAIRNYIAIGGNFHHLALLDNFCWCDSTNPKRLWQLKNAAQACYEYAKIFKTPFISGKDSMFNDFKGYNNKGEPINISAPPSLLISTVGIIENIHNAITLDVKNPGDLIYILGVTYDELGRSEYQKYSGLGNNNVPQVRAKHAKKLYKLYSNAVNTNIIASAIALNLGGLIIGLIKSLIGGELGAKIDLSLVPTHNIEDNNIKEKVILFSESQSRILVTIAPHNKQKFETIFKDIAHANIGIISDTNTLIINNMHIINLNTLKHSYKKFSNMKIQAYADAEYI

SEQ ID NO: 61 - DNA, 525 bp, Ehrlichia ruminantium (formerly Cowdria ruminantium)
  CDS (1)..(525): Corresponds to SEQ ID NO58, nucleotides 2252.. 2776 Hypothetical folic acid synthesis protein Product = “21hworf2”
atggcttactcacctgacaatgatatagtagtactagcattaggtagtaattgtggcagtatgttattgaatattaaatctgctataaatatgttatctttatataacaaaacatattcttatatctataaaagtatggcactattaccagaaaattctagtagtgattgggatactccttttctgaatatggtagtatcaggttatacaaatctttcaccaaatcttatgttagaaagagttaaatacattgaaaaaaaaataggcaggtttaataatgaatactggtcacctagatgtatagatattgacattatcttatggggagataaagtcttagactcacaaactttatctattcctcataagcatatgcaagatagagattttgtacttgtaccactctgtgatattcacgcaagatttcctcatccagtatcaaagctatcaattgaagaaatagttctcaatctacatgagatcaatttaataaagcagtcatatattataactcaatatttatag

SEQ ID NO: 62 - protein, 174 aa, Ehrlichia ruminantium (formerly Cowdria ruminantium)
MAYSPDNDIVVLALGSNCGSMLLNIKSAINMLSLYNKTYSYIYKSMALLPENSSSDWDTPFLNMVVSGYTNLSPNLMLERVKYIEKKIGRFNNEYWSPRCIDIDIILWGDKVLDSQTLSIPHKHMQDRDFVLVPLCDIHARFPHPVSKLSIEEIVLNLHEINLIKQSYIITQYL

SEQ ID NO: 63 - DNA, 560 bp, Ehrlichia ruminantium (formerly Cowdria ruminantium)
  misc_feature (1)..(560): Complement to SEQ ID NO58, nucleotides 3985.. >4544 Product = “21hworf3i”
ttattttttatcacaaaatgctacacacttcctgtaagtggtaatttttccaccacaaccagtaaaacataaatcataatcactcctacataatctatcacaatcatttaccttacataaacttacaggtttcacaggaactacagattttataccagctccaaaagattttgaagtactactactttgtacttcatgcttaaaagatcctgaagaaaaactcatatcagttttaactcgttgctcagtatcacacacattaaattgcatatcacattcagtatcacaaaaagtaccatttttacatgatttcatacatttttgtctcttatcctcacatggattattactatcttctttatcatcattacctttactgatagtattgtttattatactaataaatggattacgacgagtttgtgcatcaatcattttctctgctaatcgacaatctgttaacttaacctgacattgcctatagcaatcattcttattgtaactacattgaagtatacatgctttgccaacttcattgtctggagcttcatagtttgatc

SEQ ID NO: 64 - DNA, 4483 bp, Ehrlichia ruminantium (formerly Cowdria ruminantium)
gatccatgctaaaatcattaacataagaaccactattattatttgatatataatttacaacatctatatattttatattttgttcatgtaaaaaagatgcactataagaatctttctcagctagaatttcactaagaatatctgcaccagttatttcacttataccagaactatgagcatgtataatagctctatgaactaacctttcaaaaataatagtaggcttaacttctgagatacctttatcaattagagtaggtatttcatatcttaaaaaatttataatagtaatttttagcttatcaatagatacacgaaatgcataaaaaacacgtctagcatctatatcatccgttaacgccaataaaagatgttctaaagtagcatattcatgatgaaaatcaaacgcaattgataatgccttatgcaaactatcttcaagattctttgataacactaatgtccctaatctatattttcagacactctatcactgaaaaattaaaaataagttgcaaatgataatacatcaatttcataatcaatactgtgcaataaatttttaatattgtacttataaaaaacatatgttacacaatgatgcttaaactaactacttatatatgtttgaaaacgatatattcaagtttttcacactactactattagaaatcattttaggtatagataatgtgatttttatatcacttgccgttataaaagtaccagataccttacgcaacaaagtaagatatataggactagcattagcattaataatgcgactcgttgcattacagacagcatcgatattattgtcactaaataaaccagtaatattcctagcacaacttcatttatcacccaataacttatttatgatatttggaggagtattcttaatatatcacagcatatgtgaaatattggatgatatttcaaaaaaagctcatgataagaatcttcataacttaaaatcaaacccttacttagtaatactacagataatattaatagacttagtattctcaatagattcaatactcactgctataggaattacatataacatttttataatccaactagtatttataatatccataatacttacaatcttattttcaaagcatatcatagaagctattacaaaatacagtaacatcaaaactatagctgtcatgtttgtcttaatattaggtatcatactagtactagatggaatacatattaaaatatcccataattatttatattttacctttatcttttctagcctcgttgaaataataaatattataaaaaagtcaagcaatagcctaatacagtaaaattaataagcataataggtaatttatttaattcattatacaatgatatctctaaaaatttatacacaaattgtaaagagacacatataacataaattaaaatatgttatatataccgtaatcattcacaggattatatttaatcacaatagatattactacgtacaacaattagctctaatattttactttaaatattacaattaatattgttaccaatataattatattactaaaattaaaattttgttttgtttgtaattttgattttaagtatttttattagatgtttaaagagtattgacaatattacttatttactttaaaaaaaagggttttgacaatttttagtttctaagtagacatcaatatgtctaaaacagaaatggaatataaatctttttttatacaaagaatactacaactatagtggtaagttcagtttacattgctaaagattgttatttttacaattaaatgcctttaattatatatattaccgtatatggttattattattttacagtatacaaattctttacacataaaaattttaaagttttttgttattatttattaataaactaatagaagtacaatatattctgtatttaacttcttttaaatttgatattctaataaattgataaaagtatattatacttaaactttgtgatacatatcctttgcctaattataaaaaatgattttttattgaaatagttaatatgttattcaaaatatttgaataacatgtaagaatgattgtatattaaagatatacaatcattagttcataaagtatattcgtacctttaatggttaataatataatgtatttacatattagatatcatttataataacatacaaaaatgcttcaataataataaatctacacaatacatactactgtatttattaaatgaacatatataacaaattactgttataacttaacataatagcaataatattaaccaacactccacagaaaaacttaactatacacagttactgcttgattactattgattactattttgttacaatacacataacattttactaattaactgataactctagcgttgcgaaattatcataaatatcattatttaatataacaagaataacacataataatattaacaatgtacactagaaataataactaacaagatataaaagaaattatttagattctaataattggtttactcttactaaattatcaaaatgataatcaattttaaaatataatttcggtacatatctaagatctacatatgaaaatatagcctttcttattaaaaatgaaacatcgtttagttcttttacaagattttctttatctggatgatcatcggaaattacaacaaatacagtagcattttttacatctttactcacttctactttagatacattaactatactacaacctattgaataaatatcatgtatcaatactctcgatattgctctgcttaatactgaagcaacctttaaatttctaaaactctcagatttataaatcattgtataactctaatttcttctactatctcaaatatattcataacatcactttctggatatatctcttttgaataatcaagtaaaataccacattcaaagccagcagtaacttcctttacatcatctttaaatcgacgtaagactttaatcttaccctcatgcataatattattattacgaactaacttaactaatgcacctttttttaccaaaccacttgttacataacacccaagtacactaccgttattacctacagaaaacacttttctcacagacaaagtacctatctgtacctcttgtttcaatggcctcaacataccagttagtatcttcttaatatcatctattatatcgtatataacaaaataatgctttatttcgatatttttctgttttgccaattcttttacttgcgtatccgttttaacattaaatgctaaaattattgaatttgatgtttccgctaataaaacatctgactttgtaatattccctacacctttatatagaatattaactcgtatatctttatgagtaattttaccaattgaataacatatagcttctatagaacccataacatcacactttaagataacgttcaactcatcaaccatatcataaagcaatatattactcttatcgattgctggctgtttactcaattccacatttagtaaatcttgcctataattaattaattcacgtgcttgtttttcagaatctacaacaataaaactagtaccaaaatttgggacattatttaaaccaaataccttaattggcattgaaggaatagcaactttttcactcccaccatctgcattaaacatactacgcaccctaccataagcttgattacctgcaacaataatatcaccaactttcaaggtacctttttgtactattaacgtagcaactactccacaattcttatcaacttttgattcaattactgtaccagatgccctagtattatatacagcttttaactctaacaaatctgcaatcaacaatatacttgattttaactgatctaagtttattttctcctttgctgatacaggaacaacaataacatcacctcctaaactttctgctaccactccatgctgtaataaagcattagtaattctatctaaatcagcatcatgtttatcaattttattaaccgcaactatcatagcaacattagctgcctttacatgattaatagattcaatagtttgtggcataataccatcatcagcggccactactagcactactatatcagtaacattagtaccatgagctctcatagcagcaaatgcttcatgtcctggtgtatcaataaaagttatcttcttatctccattcaatgtgatctggtacgcacctatatgttgtgttatccctttaaattccccatcaacaacgtttgattcacgtatagcatcaagcaatgaagtttttccatgatcaacatgtcccataacagtaacaactggtgctctaggaatcaattccatattattaccatcagaatataaatcattttctaatttagcattgtctaccaacttaaatgtatgattgaacgcttctactataatagaagcttgatc

SEQ ID NO: 65 - DNA, 708 bp, Ehrlichia ruminantium (formerly Cowdria ruminantium)
  CDS (1)..(708): Corresponds to SEQ ID NO64, nucleotides 624.. 1331 Hypothetical transmembrane protein Product = “23hworf1”
atgtttgaaaacgatatattcaagtttttcacactactactattagaaatcattttaggtatagataatgtgatttttatatcacttgccgttataaaagtaccagataccttacgcaacaaagtaagatatataggactagcattagcattaataatgcgactcgttgcattacagacagcatcgatattattgtcactaaataaaccagtaatattcctagcacaacttcatttatcacccaataacttatttatgatatttggaggagtattcttaatatatcacagcatatgtgaaatattggatgatatttcaaaaaaagctcatgataagaatcttcataacttaaaatcaaacccttacttagtaatactacagataatattaatagacttagtattctcaatagattcaatactcactgctataggaattacatataacatttttataatccaactagtatttataatatccataatacttacaatcttattttcaaagcatatcatagaagctattacaaaatacagtaacatcaaaactatagctgtcatgtttgtcttaatattaggtatcatactagtactagatggaatacatattaaaatatcccataattatttatattttacctttatcttttctagcctcgttgaaataataaatattataaaaaagtcaagcaatagcctaatacagtaa

SEQ ID NO: 66 - protein, 235 aa, Ehrlichia ruminantium (formerly Cowdria ruminantium)
MFENDIFKFFTLLLLEIILGIDNVIFISLAVIKVPDTLRNKVRYIGLALALIMRLVALQTASILLSLNKPVIFLAQLHLSPNNLFMIFGGVFLIYHSICEILDDISKKAHDKNLHNLKSNPYLVILQIILIDLVFSIDSILTAIGITYNIFIIQLVFIISIILTILFSKHIIEAITKYSNIKTIAVMFVLILGIILVLDGIHIKISHNYLYFTFIFSSLVEIINIIKKSSNSLIQ

SEQ ID NO: 67 - DNA, 348 bp, Ehrlichia ruminantium (formerly Cowdria ruminantium)
  misc_feature (1)..(348): Complement to SEQ ID NO64, nucleotides 2526.. 2873 Hypothetical ribosome-binding factor A Product = “23hworf2”
ttatttagattctaataattggtttactcttactaaattatcaaaatgataatcaattttaaaatataatttcggtacatatctaagatctacatatgaaaatatagcctttcttattaaaaatgaaacatcgtttagttcttttacaagattttctttatctggatgatcatcggaaattacaacaaatacagtagcattttttacatctttactcacttctactttagatacattaactatactacaacctattgaataaatatcatgtatcaatactctcgatattgctctgcttaatactgaagcaacctttaaatttctaaaactctcagatttataaatcat

SEQ ID NO: 68 - DNA, 1614 bp, Ehrlichia ruminantium (formerly Cowdria ruminantium)
  misc_feature (1)..(1614): Complement to SEQ ID NO64, nucleotides 2870.. >4483 Hypothetical translation initiation factor IF-2 Product = “23hworf3i”
tcattgtataactctaatttcttctactatctcaaatatattcataacatcactttctggatatatctcttttgaataatcaagtaaaataccacattcaaagccagcagtaacttcctttacatcatctttaaatcgacgtaagactttaatcttaccctcatgcataatattattattacgaactaacttaactaatgcacctttttttaccaaaccacttgttacataacacccaagtacactaccgttattacctacagaaaacacttttctcacagacaaagtacctatctgtacctcttgtttcaatggcctcaacataccagttagtatcttcttaatatcatctattatatcgtatataacaaaataatgctttatttcgatatttttctgttttgccaattcttttacttgcgtatccgttttaacattaaatgctaaaattattgaatttgatgtttccgctaataaaacatctgactttgtaatattccctacacctttatatagaatattaactcgtatatctttatgagtaattttaccaattgaataacatatagcttctatagaacccataacatcacactttaagataacgttcaactcatcaaccatatcataaagcaatatattactcttatcgattgctggctgtttactcaattccacatttagtaaatcttgcctataattaattaattcacgtgcttgtttttcagaatctacaacaataaaactagtaccaaaatttgggacattatttaaaccaaataccttaattggcattgaaggaatagcaactttttcactcccaccatctgcattaaacatactacgcaccctaccataagcttgattacctgcaacaataatatcaccaactttcaaggtacctttttgtactattaacgtagcaactactccacaattcttatcaacttttgattcaattactgtaccagatgccctagtattatatacagcttttaactctaacaaatctgcaatcaacaatatacttgattttaactgatctaagtttattttctcctttgctgatacaggaacaacaataacatcacctcctaaactttctgctaccactccatgctgtaataaagcattagtaattctatctaaatcagcatcatgtttatcaattttattaaccgcaactatcatagcaacattagctgcctttacatgattaatagattcaatagtttgtggcataataccatcatcagcggccactactagcactactatatcagtaacattagtaccatgagctctcatagcagcaaatgcttcatgtcctggtgtatcaataaaagttatcttcttatctccattcaatgtgatctggtacgcacctatatgttgtgttatccctttaaattccccatcaacaacgtttgattcacgtatagcatcaagcaatgaagtttttccatgatcaacatgtcccataacagtaacaactggtgctctaggaatcaattccatattattaccatcagaatataaatcattttctaatttagcattgtctaccaacttaaatgtatgattgaacgcttctactataatagaagcttgatc

SEQ ID NO: 69 - DNA, 3829 bp, Ehrlichia ruminantium (formerly Cowdria ruminantium)
gatccagaaaattcagtgctattttcacagtcttttattccagcacatacagagttactatggatattcagttgcattacttcaacaggtcaactaaatagaatgactcaatttaaagaaaaaagccgcaataaagtttctacagcttctttaggattgtacagctatcctgtattaatggcagctgatatattactttaccaagcaaatatagtacctgtaggcattgatcaaaaacaacacttagaattagcacgagacattgctcaagcttttaacacaaaatacaatacgcaatactttcaactgccagaaccattaattgtacaggaatcagcaaaaattatgagtttaagagacggtaaaaagaaaatgagtaaatctgatgtatcagattattcacgaattaatttagaagatagtaacgacttaattgctcaaaaaattaacaaagcaaccactgactctattgtaggttttgactttacaagtttaaacaataggcctgcagtaaagaatcttgttaatatttatgctacactttcaaatattagtatagaacaaacatgtactaacattgcaagcttcactactaaacaatttaaagaagaactaacagaattaattattaataacattgcaccaatacgacaaaaattaagagagttattagaagacatagaatatttacgaagcatattaatgacaggaaataacaaggctgcatctattgcacataagcacataatagaaattaaaaagattgcaggatattggtaataattatacaaaattcattaatactcaaagtcatatcctttggttattattgtatgtgtcatggtgtttaaaaacataaaatagtttttattaccaatatgtaaagatcaaggaaattattacaatatattaatatcaacagtctcagtatgttgagagattcatatttatttaattaaactataatcttcttgactatcatctttatatattaggccattttatataaaaaaaaagaaaagaaatcctactcattaatatctaaatattaaaagagctactacaaaataactaccataatacatctatagcaaaataaagaatccatagcatcaaaatatctatactaaattcactatccatatctagtccgcatctataatactataaaattaacactgtataacaaaatatgtagtgttaatgcctataaaattaacaatattactagaaaattaaatacccaattataatactaccaaagatatccactaaataaaagtacaataataataaacaaaaagagacatagaggaatagttatattttatatcagctactaactgttataaacatatagcttaatatatatttactcaagtccataaaatacacattctcacaagagttagtacacagtaaagagaaaaaaaagttagcacttgaagagttcacttaccaatatactattcagtttcattaaaaaaattacacaattttttttctaaatataattcagaatttactattctatatagtgattctattcacttaagcattatctatatacatagtatatcacaaatctcactttaatatcctttttacacactcatcaaatccaatactcataataaaataagttatttattcaaaatactattgaatattaacgaaaatctataggacaatataacattagatgttattaaccatttttataataaacagtatacactgttgtattactttaacttcaattatagaaaatgaaaaatagtatagaatttaaagttattattaatgctctataatcctatttataccctagtgtaaaatctaaataatatttttcttacttatcaataaaaacaataaatattaccacataacctaagcatactcttcataaacttaagtaacaatatctcattatatttatttttcaaaaataaactataagcaattattaccatctaagcttatctaaatataatttatctatactataccattataaatctgattactataaagattgaactatagtcatccaaaggtttatacttgcttaattttactttacacaaaacaacaattaaaaattatatataataaaaaatttactattataaaaggctaacaaataatcaactttacgtacatgagtaaattctttctgattatgctattttaataataaaaaatactatattttgtgagaaaaatatagtaataatatgctgtaattaacacacgaaaggatttacctcctgtatttataggagataaatccttgtacagataccacaattaaataaaacaattaattcatcttaatattatttatatggttttcattagatgccagtaaatactctttcaccactacgaccaccaaatgtaaatccttttgctacttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacagctgctccttctgggctatttgttacagttgtatcaacacctgagatcaccttatcatagcacacatttaatggatgaagattaagagaaaaattagaaccttgttgtaaaaactctgaaaaaggttccattaaatttactacaaaagaagcttgtaagctgtggtttaatacatcaagtgcaatatgtttaccagtaacaccatgaaaatctgaaagtacgtgaccattactcgtaaacataacatgatattcgccatgatgattttcatgaccttcttcatgctcatgaggatgatagccaatctccattgtaatatcaccatttgaaacagagaattgattattactatagatacttaaatcatttccaaaatcaatattgtcaattcttgttgttaaatgaagcatacaatcttctgctgttgaatgaaccatacagtaacctattagtacaatgcatatttatattatatattttagtgtgttaattttgttttaagtacaactttgtgtagtaaataagtcacactacttttcaatctctacaattacgaagatacagatgtaaattcgctattttgagaagccgtatcagtaacagatactaaattagcacttacacaatcaacattatgattgtggcaatcttctgttaatggatgaagattaagagaaaaattagaaccttgttgtaaaaactctgaaaaaggttccattaaatttactacaaaagaagcttgtaagctgtgatttaatacatcaagtgcaatatgttgaccagtaacaccatgaaaatctgaaagtacgtgaccattatttataaacataacatgatattccccatgatc

SEQ ID NO: 70 - DNA, 780 bp, Ehrlichia ruminantium (formerly Cowdria ruminantium)
  CDS (1)..(780): Corresponds to SEQ ID NO69, nucleotides <1.. 780 Hypothetical tryptophanyl-tRNA ligase Product = “26hworf1i”
gatccagaaaattcagtgctattttcacagtcttttattccagcacatacagagttactatggatattcagttgcattacttcaacaggtcaactaaatagaatgactcaatttaaagaaaaaagccgcaataaagtttctacagcttctttaggattgtacagctatcctgtattaatggcagctgatatattactttaccaagcaaatatagtacctgtaggcattgatcaaaaacaacacttagaattagcacgagacattgctcaagcttttaacacaaaatacaatacgcaatactttcaactgccagaaccattaattgtacaggaatcagcaaaaattatgagtttaagagacggtaaaaagaaaatgagtaaatctgatgtatcagattattcacgaattaatttagaagatagtaacgacttaattgctcaaaaaattaacaaagcaaccactgactctattgtaggttttgactttacaagtttaaacaataggcctgcagtaaagaatcttgttaatatttatgctacactttcaaatattagtatagaacaaacatgtactaacattgcaagcttcactactaaacaatttaaagaagaactaacagaattaattattaataacattgcaccaatacgacaaaaattaagagagttattagaagacatagaatatttacgaagcatattaatgacaggaaataacaaggctgcatctattgcacataagcacataatagaaattaaaaagattgcaggatattggtaa

SEQ ID NO: 71 - protein, 259 aa, Ehrlichia ruminantium (formerly Cowdria ruminantium)
DPENSVLFSQSFIPAHTELLWIFSCITSTGQLNRMTQFKEKSRNKVSTASLGLYSYPVLMAADILLYQANIVPVGIDQKQHLELARDIAQAFNTKYNTQYFQLPEPLIVQESAKIMSLRDGKKKMSKSDVSDYSRINLEDSNDLIAQKINKATTDSIVGFDFTSLNNRPAVKNLVNIYATLSNISIEQTCTNIASFTTKQFKEELTELIINNIAPIRQKLRELLEDIEYLRSILMTGNNKAASIAHKHIIEIKKIAGYW

SEQ ID NO: 72 - DNA, 1056 bp, Ehrlichia ruminantium (formerly Cowdria ruminantium)
  misc_feature (1)..(1056): Complement to SEQ ID NO69, nucleotides 2361.. 3416 Similar to cell surface mucin, protein contains 9-mer tandem repe at Product = “26hworf2”
ttagatgccagtaaatactctttcaccactacgaccaccaaatgtaaatccttttgctacttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacaactgatccttctgggctacttgttacagctgctccttctgggctatttgttacagttgtatcaacacctgagatcaccttatcatagcacacatttaatggatgaagattaagagaaaaattagaaccttgttgtaaaaactctgaaaaaggttccattaaatttactacaaaagaagcttgtaagctgtggtttaatacatcaagtgcaatatgtttaccagtaacaccatgaaaatctgaaagtacgtgaccattactcgtaaacataacatgatattcgccatgatgattttcatgaccttcttcatgctcatgaggatgatagccaatctccattgtaatatcaccatttgaaacagagaattgattattactatagatacttaaatcatttccaaaatcaatattgtcaattcttgttgttaaatgaagcatacaatcttctgctgttgaatgaaccat

SEQ ID NO: 73 - DNA, 300 bp, Ehrlichia ruminantium (formerly Cowdria ruminantium)
  misc_feature (1)..(300): Complement to SEQ ID NO69, nucleotides 3530.. >3829 Similar to 26hworf2 Product = “26hworf3i”
ttacgaagatacagatgtaaattcgctattttgagaagccgtatcagtaacagatactaaattagcacttacacaatcaacattatgattgtggcaatcttctgttaatggatgaagattaagagaaaaattagaaccttgttgtaaaaactctgaaaaaggttccattaaatttactacaaaagaagcttgtaagctgtgatttaatacatcaagtgcaatatgttgaccagtaacaccatgaaaatctgaaagtacgtgaccattatttataaacataacatgatattccccatgatc

SEQ ID NO: 74 - DNA, 4460 bp, Ehrlichia ruminantium (formerly Cowdria ruminantium)
gatcaattattagttgcttctgtagttgattgtatggaatattttgctaacttagtcaataagcgtattactagtaaagtgaaacgttatatttctatattaagtacttctatatatgacgtgtctgttgttgagttatttaatcggcgtaacaatataatagatagaatgttgtgctaaaaggtatttttatatttgtgtaaaaggattttattaatagggttcctaattaacattggtgaattaaaacatatttttaataaattctttatatgtgtagtacatagtgaaatagtaaatcttgtgttgttataaattcatactttttcactttcaatttaataacgtcattttctggataaatagttatgaaactttctttgtatatagttagtactataggtgtgattatattgttactttgcttgatgttaattttgtattgtatcgatattgcatatgctaatattaaaaactgtgttttcaataatactgataaaactaaaaatgctgtgaatttatctattgaaaacagggttaaaaactctgttttatgtggtctaaaaaaagaatttagaagtacattaagaaatttttgtgattataacaatgttaactctgtagaagcaaaatctgctcaatatggtagtctgatggtaaaagctggttctaaatacatccaagatttaatatctgaaatagatgaccgaattgttaatcagtatattactgggagggtattatcactagaagtattaataatgcaatttgaggatacaatatatactatatgtaatgaggaaactatacagtgcgaactacaaagagtgctatatgtacgtttgcttttaaataatattttaaagttgacaaaaagtatatgtgaacaaagtgatattgaattaatggaaatatatggaatgaaatttgaatatgctttatcttttattcatagtggttttacttatataatgaaaaatatatgtacattaagtggtaatgtttattgtaataatcaaaaacagttgtgtactgatgatgttacttttactactatatcattatatgatataaaccattgtattagtcattagataaatttctaagctttatttgtatttgttatgtggaatgttcaaatattaggttaattttattcacttagataagtagtgtctattggtataaattaacttgtgctttttattctataaattgtaatagtgtatgtctaatgcgttaattcttaactatcttggtcagtgttgaattattttatttgctaatttttactgatgtgaaaagtaaatattgatatgtaagttatgtaatattatttaattactaatttcagttatgttgcattagtatgacataactgtatatttaaaattatgtattagtataattttattcaacagttttgttaaactaagatgtaatttagtttgttgtaagtgtagtatttcaatttttaatttttgatatattaatgttagctaggtaatatataatttgtatatttgatatacaaaatattagtactattgattatatacatgatcaaatttgttataattgtaagggaactaaatgagagtttcatttatgtagagaatatgattagtgttttattgtagtcaatatttcttgacatttttactagttatattttttattgtttgtgatacgttaatacaagatattagaaaatatatatagactactaattagcttatcaaaaaataaagttttatatattaaatctagtattaaagaaataaagaattataacctgatatgttaagttatgtgataaggtaacggatttaacaagagctgttttttttaacttttatgttatattaatgtattatttatatgtacatattatggatcaattggtgtatataaattaggatgatagatctcaagtatctttttatattttgttaatttatattagtttgatcttcatgctaacatcagtactagttattttagagaagtagtattaaagtacaggtttgtatagtttatttttaatttttagctgttcatattgagttaacataaatgtttaagtatgttatggcttatggaattattaaagagtattaagcttagaattttcaattttattgtttattaagttagtgtaggtactagtttggtgtcaatgtattgtagtgaagtaacgtcaggaattacaaatctccagaatttatctgttgccttactaatgcctattctgggggtgcaaatataatcatctatattgagatttgtattacaaatgcagaaactatgatttgctgtcatgtctatattgttatgttcttttgttatgtgtagggttttacatatttttcctggtccattaacttttgtatgtggtgtatttttagataataagattatacttcgaattaatattgctgcagggaaaccttcaggttctgtaacaacgttgaggcagtgatacattccatagattaaataaacataagagaatccagggttaccaaacattacagcagtgcgctttgtatatccgtggaaagaatgtgctgcttgatcatcttgtcctatatatgcttctgtttctgttataatccctttgtgttgattaaaaagtaacatcttgcctagtaagctgcttgcaacatcaagtgatttttgtttataaaatgacttctttaatatgttgtacatgtattgtctttatcactactatgggactatttacaactaattatattagtgatgtataatttttgtcaattaatggaaatgagctatggatagtgtgaagaaggggtagatattttgttaatataaaaaagctatgtgcattataatagaatacgaaatgtattttacaaaattacatttattattggtgatatctaagtataggtttaagaatatgcatagcttttcaatattaaagtaagatactataggtacaattagttagcgtataaaatattaatgctatagaaggataaattgattaaggaatatttgtagtatttaagtaacgtgactatccatatttagttatattataatggtaataattatgaaagtattgtttaatgttatctaaattataatgtttttaagttttcaagtacagttagtaatttgttggaaacatataatattcataagttatttttagtggctagttatatgatatgtgtactttgtaagagaactttagtaacattatttaattagggtaaattgatttattagcattataaagatttttataaattgtacaactattcctacattaattacctatagggttattgatttttcatattattgtgtgatatactcccatgtgttactgattaatggtgtgttatgccgtatagagattttgctataacttggttagctatattgatagcgtgtatcatcacgatatgtgtactaatacatgtgctatgtaggtatgcgtttcctgatctcaaaacacgtctagaacgggaaagaaaagcacaggcaaaaatggataagttacttgctaaacaaaacgagtcattagttaataataaacaagaagaaaagagtgaaaaagagcctgacatattgtcagaaggtgatactcagccattaggatgccattgttcaaattcagataaattgaatgatgagtctgtagagttgctagaggaacaacaggatcaactacagagtgaacagctaccacaacttgtgtctagccctactgtagttgaacaagatgagataagtcaagttgagtctacaatggaagggttacatcctactggttcaccgtgttgtcgaagacgggctctaacttctctagttagtgatgttatcattgagcaacagggtaatagccaaggtaaggagtagttttaacaggtgggttattttagtaaaggagaatcagttgttgacagttggagatctatctgttgtcaaagatagaaaatgtgttagaaaagaattacttatagaagttatataattttttttagtatctaattaagtaggattgtagatatatggtgttgttattatacaatattgttaagtctcatgaaaatgatgctatcaaattgttaatgctatcataaggtctattaaatgaaaaatctgtataacgttttatattctttttagttatgaatttatgtaatatatgtatttgaatactctaaattccatttagttaagacaaattgacttatgagaatgataagtatttagttaatgattttgctgtgatatgtattaagtgagtagcgttagacttgtctttttcattatttttgtatataatgtagtgtattagttaaataatggtctgtattatgcattctgattattacactttagctattataggttcagtaggtttagcactaatcatacttttgttatgtgttagtcaacttatcaaatatgcgatc

SEQ ID NO: 75 - DNA, 726 bp, Ehrlichia ruminantium (formerly Cowdria ruminantium)
  CDS (1)..(726): Corresponds to SEQ ID NO74, nucleotides 369.. 1094 Hypothetical outer membrane protein Product = “27hworf1”
atgaaactttctttgtatatagttagtactataggtgtgattatattgttactttgcttgatgttaattttgtattgtatcgatattgcatatgctaatattaaaaactgtgttttcaataatactgataaaactaaaaatgctgtgaatttatctattgaaaacagggttaaaaactctgttttatgtggtctaaaaaaagaatttagaagtacattaagaaatttttgtgattataacaatgttaactctgtagaagcaaaatctgctcaatatggtagtctgatggtaaaagctggttctaaatacatccaagatttaatatctgaaatagatgaccgaattgttaatcagtatattactgggagggtattatcactagaagtattaataatgcaatttgaggatacaatatatactatatgtaatgaggaaactatacagtgcgaactacaaagagtgctatatgtacgtttgcttttaaataatattttaaagttgacaaaaagtatatgtgaacaaagtgatattgaattaatggaaatatatggaatgaaatttgaatatgctttatcttttattcatagtggttttacttatataatgaaaaatatatgtacattaagtggtaatgtttattgtaataatcaaaaacagttgtgtactgatgatgttacttttactactatatcattatatgatataaaccattgtattagtcattag

SEQ ID NO: 76 - protein, 241 aa, Ehrlichia ruminantium (formerly Cowdria ruminantium)
MKLSLYIVSTIGVIILLLCLMLILYCIDIAYANIKNCVFNNTDKTKNAVNLSIENRVKNSVLCGLKKEFRSTLRNFCDYNNVNSVEAKSAQYGSLMVKAGSKYIQDLISEIDDRIVNQYITGRVLSLEVLIMQFEDTIYTICNEETIQCELQRVLYVRLLLNNILKLTKSICEQSDIELMEIYGMKFEYALSFIHSGFTYIMKNICTLSGNVYCNNQKQLCTDDVTFTTISLYDINHCISH

SEQ ID NO: 77 - DNA, 567 bp, Ehrlichia ruminantium (formerly Cowdria ruminantium)
  misc_feature (1)..(567): Complement to SEQ ID NO74, nucleotides 2149.. 2715 Hypothetical DNA-3-methyladenine glycosidase Product = “27hworf2”
ttaagttagtgtaggtactagtttggtgtcaatgtattgtagtgaagtaacgtcaggaattacaaatctccagaatttatctgttgccttactaatgcctattctgggggtgcaaatataatcatctatattgagatttgtattacaaatgcagaaactatgatttgctgtcatgtctatattgttatgttcttttgttatgtgtagggttttacatatttttcctggtccattaacttttgtatgtggtgtatttttagataataagattatacttcgaattaatattgctgcagggaaaccttcaggttctgtaacaacgttgaggcagtgatacattccatagattaaataaacataagagaatccagggttaccaaacattacagcagtgcgctttgtatatccgtggaaagaatgtgctgcttgatcatcttgtcctatatatgcttctgtttctgttataatccctttgtgttgattaaaaagtaacatcttgcctagtaagctgcttgcaacatcaagtgatttttgtttataaaatgacttctttaatatgttgtacat

SEQ ID NO: 78 - DNA, 240 bp, Ehrlichia ruminantium (formerly Cowdria ruminantium)
  misc_feature (1)..(240): Complement to SEQ ID NO74, nucleotides 3369.. 3608 Product = “27hworf4”
ttaatggtgtgttatgccgtatagagattttgctataacttggttagctatattgatagcgtgtatcatcacgatatgtgtactaatacatgtgctatgtaggtatgcgtttcctgatctcaaaacacgtctagaacgggaaagaaaagcacaggcaaaaatggataagttacttgctaaacaaaacgagtcattagttaataataaacaagaagaaaagagtgaaaaagagcctgacat

SEQ ID NO: 79 - DNA, 519 bp, Ehrlichia ruminantium (formerly Cowdria ruminantium)
  CDS (1)..(519): Corresponds to SEQ ID NO74, nucleotides 3382.. 3900 Hypothetical lipoprotein Product = “27hworf3”
atgccgtatagagattttgctataacttggttagctatattgatagcgtgtatcatcacgatatgtgtactaatacatgtgctatgtaggtatgcgtttcctgatctcaaaacacgtctagaacgggaaagaaaagcacaggcaaaaatggataagttacttgctaaacaaaacgagtcattagttaataataaacaagaagaaaagagtgaaaaagagcctgacatattgtcagaaggtgatactcagccattaggatgccattgttcaaattcagataaattgaatgatgagtctgtagagttgctagaggaacaacaggatcaactacagagtgaacagctaccacaacttgtgtctagccctactgtagttgaacaagatgagataagtcaagttgagtctacaatggaagggttacatcctactggttcaccgtgttgtcgaagacgggctctaacttctctagttagtgatgttatcattgagcaacagggtaatagccaaggtaaggagtag

SEQ ID NO: 80 - protein, 172 aa, Ehrlichia ruminantium (formerly Cowdria ruminantium)
MPYRDFAITWLAILIACIITICVLIHVLCRYAFPDLKTRLERERKAQAKMDKLLAKQNESLVNNKQEEKSEKEPDILSEGDTQPLGCHCSNSDKLNDESVELLEEQQDQLQSEQLPQLVSSPTVVEQDEISQVESTMEGLHPTGSPCCRRRALTSLVSDVIIEQQGNSQGKE

SEQ ID NO: 81 - DNA, 560 bp, Ehrlichia ruminantium (formerly Cowdria ruminantium)
  misc_feature (560)..(560): n = a, c, g, or t
gatccataatctcatcaataacccctgtaattgaggtattagtagaaatattagctatatcacaccaagcattttcaataatttctttaaaatcagaagtatttaccatattcacatacctcaacttaacaacacagatttattattataaacactattaaaaaaataacaagatacacactatgaatcagataaccttggtattctaatatacgtatgatataatgatactaaaccataagtttatattatttaaagacataatgacatatctttaaatgctaatatgtataatcttaaagtccttaagatacatacatttatagacatatctgtaataatgcactatataatgttaagatgtatagtcaataagtttgtgtttaatgagaatagaatgcaaaaattgcaaagcagtttatagaatagacaatagcaaaattcccattaatggtaaaaaagttaaagttaaatgcacaaactgtaatactacatggatgcacataccaactcaagataaagcaatacctgaagaagaaaaacaattagtaataggatcn

SEQ ID NO: 82 - DNA, 174 bp, Ehrlichia ruminantium (formerly Cowdria ruminantium)
  CDS (1)..(174): Corresponds to SEQ ID NO81, nucleotides 387.. >559 Product = “1gdorf1i”
atgagaatagaatgcaaaaattgcaaagcagtttatagaatagacaatagcaaaattcccattaatggtaaaaaagttaaagttaaatgcacaaactgtaatactacatggatgcacataccaactcaagataaagcaatacctgaagaagaaaaacaattagtaataggatcn

SEQ ID NO: 83 - protein, 58 aa, Ehrlichia ruminantium (formerly Cowdria ruminantium)
  misc_feature (58)..(58): The ′Xaa′ at location 58 stands for Ser.
MRIECKNCKAVYRIDNSKIPINGKKVKVKCTNCNTTWMHIPTQDKAIPEEEKQLVIGX

SEQ ID NO: 84 - DNA, 2008 bp, Ehrlichia ruminantium (formerly Cowdria ruminantium)
gatcaaaaagggataccaaaagagcaatactatataaaattagtatctgttaattcaacatatcctgactcaattaataacttactcatatttagtagtattattgaaagctatgaaggcactgtgaacaaatttcgttgtggagatactgtaagtataaagtacgatatacgtgaactaaacggtaatacgatactacaagatcaagaattaaaatttactattggaaaaaatgaagtccctcttgcaatagagctaggtgtaattaatatgagacaagacatggcaagacatattattgcaccattagaacttttgactaattttgacaaacctgacaacttttgatgaatacaaaataaaactaattgatattacctacattaatcaaccacaacctatacaaaaaaacgcaaagcccagccaatcttaaggtgattatttcttatatctgtatagccacataaaaaagctaaaaataacgttattatatcaaataaattacaatcaacaatactacactaatattatagaaatctactaatatattgatatagtaaaataatacacatttacacaatcaatacttaaattcataataacttgtgtcaactttataaaaccagctatttcataaaaataacacaaaactataaaacaccaaatagcttactcacgctaaatgtttctataaataaatcaacttgttattgtaataatataaaaactcaccaattttaataaaacaaatatatactaatcttttatttcctaatttatcttaataagattcaatatccttatactaatacaaatcttaactcatatattaccaccccacactaacaaatccatagaacttgctaagtgtatattatataatattaatttaacatgttaatgactgaaaaagtattaacttaaagatctatttaataaaatttaacctcttctataaccttaactatcatcatttttaagtaactgaagtatttaagacatttaacaattatatatcatataaaaatctttaatgtactagcaattgatgaattatgccctcataatatatgcaagcataaaatgcctattttaacaaaactttatctattctataaccttaactattaccatttttaagtaactgaggtatttaagacatttaataattatataccatataaaaatctgcttaatgtactagcaattgatgaattattccctcataatatatgcaagcataaaatgcctaatttaacaaaactttatctattctataaccttaactattactattttcaagtaactgaagtatttaagatatttgacaattatatatcatattaaaatctgcttaatgtactagcaattgatggattattctctcataatatatgtaagcataaaatgcctaatttaataaaactttatctattctataacttaactattactattttcaagtaactgaagtatttaagatatttgacaattatatatcatatcaaaatctgcttaatgtactagcaattgatggattattctctcataatatatgtaagcataaaatgcctaatttaataaaactttatctattctataaccttaactattactattttcaagtaactgaagtatttaagatatttgacaattatatatcatataaaaatctttaatgtactagcaattgacaactatgccctgataatctatgtagcataaaatacctcatttaacaaaactttatctattctataaccttaactattaccatttttaagtaactgaagtatttaagatatttgacaattatatatcatataaaaatctttaatgtactagcaattgacaactattccctgataatctatgtagcataaaatacctacaggaatctttattaatagtaatttacttatctattgaacataactttaagtattactagtcataatattataaagacacatttaatattaaacattgtagacattttaacatgatc

SEQ ID NO: 85 - DNA, 348 bp, Ehrlichia ruminantium (formerly Cowdria ruminantium)
  CDS (1)..(348): Corresponds to SEQ ID NO84, nucleotides <1.. 348 Product = “2gdorf1i”
gatcaaaaagggataccaaaagagcaatactatataaaattagtatctgttaattcaacatatcctgactcaattaataacttactcatatttagtagtattattgaaagctatgaaggcactgtgaacaaatttcgttgtggagatactgtaagtataaagtacgatatacgtgaactaaacggtaatacgatactacaagatcaagaattaaaatttactattggaaaaaatgaagtccctcttgcaatagagctaggtgtaattaatatgagacaagacatggcaagacatattattgcaccattagaacttttgactaattttgacaaacctgacaacttttga

SEQ ID NO: 86 - protein, 115 aa, Ehrlichia ruminantium (formerly Cowdria ruminantium)
DQKGIPKEQYYIKLVSVNSTYPDSINNLLIFSSIIESYEGTVNKFRCGDTVSIKYDIRELNGNTILQDQELKFTIGKNEVPLAIELGVINMRQDMARHIIAPLELLTNFDKPDNF

SEQ ID NO: 87 - DNA, 3829 bp, Ehrlichia ruminantium (formerly Cowdria ruminantium)
gatctacaaatgaaaagtgtatggttaactttacaataatggattggtgaaagtaaatttgacaaattaatcaataccttaaaatgagttgagtggaaaagtataaataatgtaggtgttttttttgtttataattgaattaaagtgcttaaagatatttatatagttattacttagattatttaattaaattaagctagtgaacaaagaatttgtattatatatattttaggtatctattatctttacagtttaaataagaaagcaagggtttattaattatatagtgaatgaaatatattattactgagtatatcatgtttattatatacattattcactattcacaaattcagtgtatgtgataaacctaaataattatacaaatattagggggggggggggtatattttttcgtaagagcttacattaatgtaattattaatagtattttttgtttttaaaagatgtaaattctactgttgtttgttactgcataacaatattcagtgataaacaaggatttacaataaatacacttgcctttttaagaatattttagtagttttgagaatcatatgatacttgatgatgagtatacctaatgagtagttcgttacctgttttacaggttattatacctttactttctgcagtaatatgtgctttattaaaaaatagtacattggtgaaaattatatcatctattgttgtagtagtatctttttccattgcgttggtattgttctctcaggtttattctgctgatgtaatcaaatacagcctaggggggtgggtcgttccttatggcatagaacttaaagtgaacatatttagtgctactatgcttgttttagtaaattttattgctgtaatgagtatattgtatggcatatatcctaacatcagagaaataggtgttaacaagataccaagtttctattctgtatttttgctatgtttaggtggctttttggggatattagtatcaaatgatgtttttaatatctatgtttttcttgagatttcgtctatttcttcttatattttggttgcaatgggaaaagataaagctgctttaatagcagcatttgattatttagtaattggtacaattggggcaactttttatttaataggtataggctttttgtatgctattactggtacattgaatattggagatttgtttctaataattcatgataatttgctggtaacaaatagagttacacagattgcaatgttatttattatggtaggtttgtttataaagacagcgctattcccatttcataaatggttaatacaggcttatagttttgctccttcttttatttctgtgtttttttccggtacttctactaaagttatgatatatctaattataaagatgatatatgacgtttttaaagctgattttgtttttgtgactttaccttttaatattgtttttatgtgttttgctgtgttgtcaatagtttgtggatctttacttgcaatttttactagcaatattaaaaagatatttgcttattcaagtattgcacatttaggatatattgtatttgcagttagtttaaatactaattatggtttggttgcagctatagcttatattattagtcatagcttggttaagtcagcattatttatgattgtaggtagcattgattatagttgtggtaacagacatctgaaagattgtgcaaacatgtgggaaaccatgccaaaaattacattgccatttattatattatgtttaagtttaattggtatgccagttacttcagggtttattgctaaatggtatattgttgatgcagttataaagtctaatttttgggttggtatttttgtgttgcttataggttctgggttatctatagtgtatgtttggaaaatagttgaagcagtgtgtcttcgttcacctgataataaggtagttatgtcgtcgtttgaaacaccaaatgttatggtattatgtatttggataatggtaattgcttcgattattgtgggaatatatccaattcctttaacattgatttctaataaaatagcgacgttgctattatattgagatatctatttgaattttattattactaaatggtttatttagatggtgagttacactcaagtaataataaaaaatgtcaattaactacccattcctaggtaatttatacctaactattaactaatcattcttatgaggaaaatgtagtaaacattactaccttacataataactatgtaaaaatctaaagaaataccaagatagtacctcctcttaatataaagaagttatctacttctactttgttctttactttcaaatacttgagatactgagtcaatagtaaaagttgtactgtgtgcaacatgatgttctggagtataattcctatcaaatggtttatccatactacctatattttcttgagtacttacagatgattgatctttaggttgaagctttttcttaagttttttattgtacttaaagaaataagtactacctatcactaatagaagtattagtaacgctattgactttgggttactaactatatataataataagttataaaaatcatttagcataacattatcttaaataaccataacacaactatcattatattccaaaaatgcatataagcatagtaaataattaatgctatagcatgttttttgttttgcctgtaaagttaagttgtaatcattaaataagtttattttcactttgtaatatattactgtatttgcttagttttcgtataattctaccacttacttcaatcagccttatatttactggtaataatgcaattcttccttaatattaatagttaatataaataaattaatacttaattataacataagtttaagaatattactagtaatgtattgctgttgtttgatttttatgtctattatcagtttagcatattatgctgttagaaatatattttaataacactattgagtaaatgaaactattatctatgtaaaaaaattatgttgatgcttaaatctaatgtgtatcttataattgagcttttattgatggtaattacttaaattttatgtaaactcaataatgtcaaatatttatattatctttatttagttgttcatcctattagttagttataatatgcttgttgtcaaatttgtatttaacaattgatattgactacttaataagtgaatctattattttagcaataattgcattgaaacttaaatctgaatgtgtatgttctattaaaccttcacttacgatgacttcttgatctgatccaagttcgatatcaatgtgcttttttcctaacatattgctgtttaatatagatgctgaactatctgaaggtagtaagatatttttctgtatgcacattgttactataggagtatagctttcattcaatgatattgaagttactgtacctatttttactcctgatattgttacttcatctcctatgtccaacccatctacatttgagaaaaatgctttaactgtataacaattacgcaaggtatttttatatggtaatttgttaaatgctattatcccaatagatattgctcctgctaacactaggaatcctataaaaatttcaataatatttgatctatgcataaaataccgatgttaatatgttttgtgttatttaaaagacttttgaggatggtaggcgtacttagttccggttaaatttggtacatgtagatttttttctgcaagtgttggtaattgattatctgtataatgtaaccatatatgccataatgcaggaacttttgttggatc

SEQ ID NO: 88 - DNA, 1479 bp, Ehrlichia ruminantium (formerly Cowdria ruminantium)
  CDS (1)..(1479): Corresponds to SEQ ID NO87, nucleotides 605.. 2083 Hypothetical NADH dehydrogenase (ubiquinone) Product = “3gdorf1”
atgagtagttcgttacctgttttacaggttattatacctttactttctgcagtaatatgtgctttattaaaaaatagtacattggtgaaaattatatcatctattgttgtagtagtatctttttccattgcgttggtattgttctctcaggtttattctgctgatgtaatcaaatacagcctaggggggtgggtcgttccttatggcatagaacttaaagtgaacatatttagtgctactatgcttgttttagtaaattttattgctgtaatgagtatattgtatggcatatatcctaacatcagagaaataggtgttaacaagataccaagtttctattctgtatttttgctatgtttaggtggctttttggggatattagtatcaaatgatgtttttaatatctatgtttttcttgagatttcgtctatttcttcttatattttggttgcaatgggaaaagataaagctgctttaatagcagcatttgattatttagtaattggtacaattggggcaactttttatttaataggtataggctttttgtatgctattactggtacattgaatattggagatttgtttctaataattcatgataatttgctggtaacaaatagagttacacagattgcaatgttatttattatggtaggtttgtttataaagacagcgctattcccatttcataaatggttaatacaggcttatagttttgctccttcttttatttctgtgtttttttccggtacttctactaaagttatgatatatctaattataaagatgatatatgacgtttttaaagctgattttgtttttgtgactttaccttttaatattgtttttatgtgttttgctgtgttgtcaatagtttgtggatctttacttgcaatttttactagcaatattaaaaagatatttgcttattcaagtattgcacatttaggatatattgtatttgcagttagtttaaatactaattatggtttggttgcagctatagcttatattattagtcatagcttggttaagtcagcattatttatgattgtaggtagcattgattatagttgtggtaacagacatctgaaagattgtgcaaacatgtgggaaaccatgccaaaaattacattgccatttattatattatgtttaagtttaattggtatgccagttacttcagggtttattgctaaatggtatattgttgatgcagttataaagtctaatttttgggttggtatttttgtgttgcttataggttctgggttatctatagtgtatgtttggaaaatagttgaagcagtgtgtcttcgttcacctgataataaggtagttatgtcgtcgtttgaaacaccaaatgttatggtattatgtatttggataatggtaattgcttcgattattgtgggaatatatccaattcctttaacattgatttctaataaaatagcgacgttgctattatattga

SEQ ID NO: 89 - protein, 492 aa, Ehrlichia ruminantium (formerly Cowdria ruminantium)
MSSSLPVLQVIIPLLSAVICALLKNSTLVKIISSIVVVVSFSIALVLFSQVYSADVIKYSLGGWVVPYGIELKVNIFSATMLVLVNFIAVMSILYGIYPNIREIGVNKIPSFYSVFLLCLGGFLGILVSNDVFNIYVFLEISSISSYILVAMGKDKAALIAAFDYLVIGTIGATFYLIGIGFLYAITGTLNIGDLFLIIHDNLLVTNRVTQIAMLFIMVGLFIKTALFPFHKWLIQAYSFAPSFISVFFSGTSTKVMIYLIIKMIYDVFKADFVFVTLPFNIVFMCFAVLSIVCGSLLAIFTSNIKKIFAYSSIAHLGYIVFAVSLNTNYGLVAAIAYIISHSLVKSALFMIVGSIDYSCGNRHLKDCANMWETMPKITLPFIILCLSLIGMPVTSGFIAKWYIVDAVIKSNFWVGIFVLLIGSGLSIVYVWKIVEAVCLRSPDNKVVMSSFETPNVMVLCIWIMVIASIIVGIYPIPLTLISNKIATLLLY

SEQ ID NO: 90 - DNA, 300 bp, Ehrlichia ruminantium (formerly Cowdria ruminantium)
  misc_feature (1)..(300): Complement to SEQ ID NO87, nucleotides 2311.. 2610 Product = “3gdorf2”
ttatctacttctactttgttctttactttcaaatacttgagatactgagtcaatagtaaaagttgtactgtgtgcaacatgatgttctggagtataattcctatcaaatggtttatccatactacctatattttcttgagtacttacagatgattgatctttaggttgaagctttttcttaagttttttattgtacttaaagaaataagtactacctatcactaatagaagtattagtaacgctattgactttgggttactaactatatataataataagttataaaaatcatttagcat

SEQ ID NO: 91 - DNA, 450 bp, Ehrlichia ruminantium (formerly Cowdria ruminantium)
  misc_feature (1)..(450): Complement to SEQ ID NO87, nucleotides 3209.. 3658 Hypothetical outer membrane protein, related to proposed ABC transporter Product = “3gdorf3”
ctacttaataagtgaatctattattttagcaataattgcattgaaacttaaatctgaatgtgtatgttctattaaaccttcacttacgatgacttcttgatctgatccaagttcgatatcaatgtgcttttttcctaacatattgctgtttaatatagatgctgaactatctgaaggtagtaagatatttttctgtatgcacattgttactataggagtatagctttcattcaatgatattgaagttactgtacctatttttactcctgatattgttacttcatctcctatgtccaacccatctacatttgagaaaaatgctttaactgtataacaattacgcaaggtatttttatatggtaatttgttaaatgctattatcccaatagatattgctcctgctaacactaggaatcctataaaaatttcaataatatttgatctatgcat

SEQ ID NO: 92 - DNA, 226 bp, Ehrlichia ruminantium (formerly Cowdria ruminantium)
gatcaagctttaaatcatttccaatcattgataccttagtaagaacatcaggaccattacctataatagtagcacctttaataggagatgtaactttcccatcttctattaagtagctttcagaagctgaaaaaacaaatttaccagatgtaatatctacctgaccaccagcaaaattcacagcataaattcctttcttcacactagcaatgatttcatttggatc

SEQ ID NO: 93 - DNA, 226 bp, Ehrlichia ruminantium (formerly Cowdria ruminantium); identical to SEQ ID NO: 92
  misc_feature (1)..(226): Complement to SEQ ID NO92, nucleotides <1.. >226 Hypothetical tldD protein Product = “4gdorf1i”

SEQ ID NO: 94 - DNA, 160 bp, Ehrlichia ruminantium (formerly Cowdria ruminantium)
gatctgcacgagttcttcgtccgtggtgaagttgcagtggacatgcaggatgtccgggccgagcagcccggcgtggccgagctgggcgacgccgtcgggcgtcgacagcgtgcccatatgcgccgtgatcggcaggccgtgggcgcgggcgaactcgatc

SEQ ID NO: 95 - DNA, 299 bp, Ehrlichia ruminantium (formerly Cowdria ruminantium)
gatctatggaaaacatccttgtgttgctgcactacaaaatataaatagaaaatgtcatgagcttttagttacagaaaatttcattaaacataacaatggaatacaaaaaattagagagcttagtaaacaaaaaaatatttacccaaaacaagtaaatatcaatactatcaactcagtactaccacctaatagtaaccaccaaggcattgctttacaagtttcaatagtagatacagtaagcatagaagacgtattatctaatattcctacagagatttcaacaataatacttttagatc

SEQ ID NO: 96 - DNA, 297 bp, Ehrlichia ruminantium (formerly Cowdria ruminantium)
  CDS (1)..(297): Corresponds to SEQ ID NO95, nucleotides <1.. >297 Product = “6gdorf1i”
atctatggaaaacatccttgtgttgctgcactacaaaatataaatagaaaatgtcatgagcttttagttacagaaaatttcattaaacataacaatggaatacaaaaaattagagagcttagtaaacaaaaaaatatttacccaaaacaagtaaatatcaatactatcaactcagtactaccacctaatagtaaccaccaaggcattgctttacaagtttcaatagtagatacagtaagcatagaagacgtattatctaatattcctacagagatttcaacaataatacttttagat

SEQ ID NO: 97 - protein, 99 aa, Ehrlichia ruminantium (formerly Cowdria ruminantium)
IYGKHPCVAALQNINRKCHELLVTENFIKHNNGIQKIRELSKQKNIYPKQVNINTINSVLPPNSNHQGIALQVSIVDTVSIEDVLSNIPTEISTIILLD

SEQ ID NO: 98 - DNA, 2104 bp, Ehrlichia ruminantium (formerly Cowdria ruminantium)
gatccgtatgctaaaaggtctaaatatagacaattaggaattaattgaagagatatattatcaatatttgtaatatttggaatagttgctatggtaactgtttgtcaaatacatagtgtgcattttaccagaatgatgaataatgacatttcataaagtttaataaataaggatttataatgaatgctaaagaaaaaaatattagagaagaaattttaaatcttcaaaaaaagattgctgaatgagacaatgcttattataatttggataatcctattgtaactgatgaaatttatgatactgagtttattagacttcaaaaattagaaaaacaatatagtcatttgctaacttatgaagaagttaaaaactctccaacgcaaaaaattgatgcaaagtctttatcaatatttgataaagtaattcataaaaaaccaatgctttctttaaacaaagcatattcaattgaagaaattaagaaatttattaaaaagattgaaaaatatactaatgatttttcattttttattgaacctaaaattgatggtctttctatttcattaacttatgaaaatggaaaactaattagaggtgtaactagaggagatggaataacaggagaagatgttacaaaaaatattttacaaattaatgatatccctaaagaaatagaatacaaacacaaaatcgaattaagaggaaaaatatatttatctatttctagatttaatgaattaaatgaagaaaatttaaaaaataatttaccgcctttagctaacccaagaaatgcagcagccggaactttaagacaattagattctaatattgtttctcaaagagggttgtcatcttttatatattttgtagtcgatgctcccagtcataatatttggacaatggaagatgccttttgttttcttaagaaaaataattttcatgttgtaaaagattataaactagctaaaaatattaatcaaattgaagaatatataaataattttccagaacttaaaaaaacatttgattttgaagcagatggtgtagttattaaattaaatgaaattaaatgatgaaataaaattggacaaactcaaaagtttccacattatgcgattgcttttaaatttgaaccaaacattgaaattacaacaattaaaaagatatttataactattggaagaactggcctagtcacttataatggccaagttaaaactgtcgaaatttctggttccaaaataaactttgcaacattaaataattttaattacgttaaagaactaaatttaaatgttggcgatgaagtttatattaaaaaggctggtgaaattattccttgcattataggactagttaatccaaaaggtaaacctgattattttaaaagaatagaaacttgcccatattgtaattctaaattaatagaaagtgaaacattcttagaagaatattgtgaaaattacaattgtccagaaattataaaaaaacaactaattcatttttcttcaaaagaatgtatgaatttcttttcaatgggcgaaaaaatagtagaaaaattatatgaaaataaattaattcttagtccactagatttttataatttaaaaaataataaaaatgaactaacacaattagaaaaattaggaactaaatctataatgaaaattttagattcaattgaagattcaaaaaaattaggacttgacaaatttatttttgctttatctataaaacacataggacaaaaagttgcaagttttataacttctaaagttcaaaaactttctgagtttctaacttttgattttgattctttaattcaatataatgaaattggtccaaaaattattgattcagttaaaaaatgactatcagctgaaaataataaaaaattaattaatgactttcttaatagaggaatgaatttcgaacatatttcaaatataaaaagcaaattattagatggaattaatattgttattacaggaacattatctaagcctagaaattattttgaagaattaataaaagcaaataacggaaatatagtaaatagtgtatctaaaaaaacttcttatgttttatgcggaaaaaatcctggatc

SEQ ID NO: 99 - protein, 642 aa, Ehrlichia ruminantium (formerly Cowdria ruminantium)
  MISC_FEATURE (1)..(642): Corresponds to SEQ ID NO98, nucleotides 180.. >2104 Hypothetical DNA ligase Product = “7gdorf1i”
MNAKEKNIREEILNLQKKIAEWDNAYYNLDNPIVTDEIYDTEFIRLQKLEKQYSHLLTYEEVKNSPTQKIDAKSLSIFDKVIHKKPMLSLNKAYSIEEIKKFIKKIEKYTNDFSFFIEPKIDGLSISLTYENGKLIRGVTRGDGITGEDVTKNILQINDIPKEIEYKHKIELRGKIYLSISRFNELNEENLKNNLPPLANPRNAAAGTLRQLDSNIVSQRGLSSFIYFVVDAPSHNIWTMEDAFCFLKKNNFHVVKDYKLAKNINQIEEYINNFPELKKTFDFEADGVVIKLNEIKWWNKIGQTQKFPHYAIAFKFEPNIEITTIKKIFITIGRTGLVTYNGQVKTVEISGSKINFATLNNFNYVKELNLNVGDEVYIKKAGEIIPCIIGLVNPKGKPDYFKRIETCPYCNSKLIESETFLEEYCENYNCPEIIKKQLIHFSSKECMNFFSMGEKIVEKLYENKLILSPLDFYNLKNNKNELTQLEKLGTKSIMKILDSIEDSKKLGLDKFIFALSIKHIGQKVASFITSKVQKLSEFLTFDFDSLIQYNEIGPKIIDSVKKWLSAENNKKLINDFLNRGMNFEHISNIKSKLLDGINIVITGTLSKPRNYFEELIKANNGNIVNSVSKKTSYVLCGKNPGS

SEQ ID NO: 100 - DNA, 4055 bp, Ehrlichia ruminantium (formerly Cowdria ruminantium)
gatctattggtagttcattcacatctggaataataatctcttcattatctcttttcgaataccataccggaaacgggattccaaaatacctttgtcgcgatatgcaccagtcccagtttaacccatctatccacatttctatttgcttacgcatagactgtggataccaattaatcttacggacctgttctaatagctcatcttttatctctacaaccttaataaaccattggttacttaacaatatttcaataggcattcctgatcgctcagcacatttcacattgtgtaatatttcctctttttttatcagtaaattacatttacttaaggtttcaagtaccagctttcttgcttctactattgatactccatgtaatttaccagataaagtatctgtctctgcaatgttatgtttaagatcaagagtacctgatttacttattataatctgcgtatttaaattatgtttattccaccaatatacatctaattcatcaccaaatgtacaacacattacaagaccagtacccttatctatttttacttgttcatctgataaaatcggtactttattcccaaatataggtactatagcatactgaccttgaagatgctgatatcttatatccaatggattaaaaaataaagcaacacaagctggcattaattctggacgcgtcgttgcaatatttattagctctccagcctccgtagaaaaggctatcgtactcataaacgatgacatttccttttcctcaacctcaactctcgctatcgccgttctatcagcacaatcccaaaatataggctgtaacttcctatatattttacccatattatatagtgctataaatgacatttgagataacttttgaatctcttcacttatagtatgatattccagatcccaatcataactaataccaagagattgaaacaatattttaaattccattctgaattttgcagatacttcattacataatgccttaaattctttacgatcaatatctgtcgcacgtactttttttatcttttcaactaaacgttctgttggcaatccattgtcatcaaatcctattggatatagtacatcttttcctaacatacgttgatatcttgcaataaaatccgtatgacagtagctaaaaacatgtcctatatgtagttgtcctgatattgttggaggaggagtatcaataataaactgtttatcttgtaaattcttccatttatataattttattttatcccaataggtattaagtttttcttctgtatctttgaacttgtatttattactaaaaagagactgcattatcaatgatttatttgctttctgctttaagtatagaaaattgctataattgaaacaatgataattttacaaattgtattacattaagaataacatcatattatccatagtgatatacaatgtattttcagtagtatagaaacagtttactattgtatcattattcatatttaactaagatattaataatacttatacaaaggaatttgagataataaaagtctcattagttgtatttataacataagtcatagtatgtcattttacatttcacatatgaaacatttttgctaagtaatatcaatatacaattctttaatggtcttatataaaaactcattataacattcgttatattaaataaaaaacctataattgggatataaaatcatattacacaataacaaaacctttataagttgtatcatgtacaactcatgcaaagcttgtttagtctataatacttatactaaataacataacactactattcaattgtttcacatcagcatatatgatatgcatctactagaattagaagatatatcagtaacatcttataagaacaaaaactctacaaattacattatttaaaattaataaacaaattttttagtatagtcataactcaaaaatactctagattcataatattacacttattaactgtttcatagcagaactatatttataattaaaatataaagacaacacttaaaaacaacaaaaatctcacaattgtattatgacaaacaaaacactttcttcttaattatcacttaaactatgcactgatactagatatacaacattgtagaattaccttatatcttttacaaagataatcttagttttacgcatcttgtccattattgaaatattccgtagtttaaaatatatatttgtcttgtattatgcaattttttccttagcacattattcagctattattttaacttgagaccttatcaaattaagttcattactacaaatatatgacttgtttttttattctaactctccctgtcattatttcctttcttatcaggctactctttttaccttgtaaatgataatattaattaattatttagttatctgttcacatcattagatagcttgaaacatacatttgtactatattacatattcctctgaactcattactcaatcattatctatgatttaaaatcttattattaatcgacttaaatgatcatcatctagcaagagattaatatatactgtttttttatttcaaaccttctaagctttcattatttttctcttagaacataactttacactaatacgcactacaacatcagcaatctaaaatatgctagctaaattacacacaaaaatttcataaaaagttcatgttaccctcatttaactcatattaaataatgttaagccgtattttgttatatactacttacatgattatgataatgttactagtcttaattaattataaaaaggcagattttaaataataaacctatcacctttcacttacaatttataatacaccaaaatattcgaattactaattaatatcaacaactcaagtttaatgctaattttatcgtatactaattgtatttagagtaatactacccacatataaaaaaatacacacatataaaattataaactagagagaaaaataccacaataaatcaaaagtcacacaaccaattattcactaagattatataactctgccaactatatcaacaaaagcctaactactgataaacactatatctatatataaaattataaatctagattaaggaaagtgtaaatcatcaattaaagatcaaattactcattaatacttatcacacaaaattagattcaattatgtaattcatacctacaactcaaagctcatctaataaataaaaatttaactacaactaaaaactccctcccaccaataaaaccataaaacctatatagacaaaatactaataacaaaccaaaagctacataactgagttattcattaacattagctaccaaagtttaatcttgattcattataatatctgcctaccatacttataaaaactcaactacagttaaaaactaatctctacggaaacatagtttatacataacttaaaagagaaaataaggtaaaataatatcaataaattttaataataagctactaccgatcaaaaacttcaactgcagcagccaatgcagccatcttagcaaccatactccaacagaatattttaccttcttgctctctaaaacttacatctacaaaatcacaacctacagaatttaggttagagaatttatctttaacagtattatacctatataaataacacgaaggagtatcacctatgaaatatactaatggttctgcagtataaccattaaacaactcttcagtcattataccttcttgtattattacacgctcaactattttcctatccttaattcttttcatcttattgcgattttttttatttaacttcaatatgtcatcaccacaatatgctacaataattcccattccatatgttccattatctgcttttacaaacacatacggttgttctgtaatactgtataattgaaatttattacgtatttcttgaatcataacatcaactttatcagcaatgtgttctactccataattacttaaaaaacagatgttatcacaactagaaaataatgtagaaatcaaccaaggatc

SEQ ID NO: 101 - DNA, 603 bp, Ehrlichia ruminantium (formerly Cowdria ruminantium)
  CDS (1)..(603): Corresponds to SEQ ID NO100, nucleotides <1.. 605 Product = “3hworf2i”
tctattggtagttcattcacatctggaataataatctcttcattatctcttttcgaataccataccggaaacgggattccaaaatacctttgtcgcgatatgcaccagtcccagtttaacccatctatccacatttctatttgcttacgcatagactgtggataccaattaatcttacggacctgttctaatagctcatcttttatctctacaaccttaataaaccattggttacttaacaatatttcaataggcattcctgatcgctcagcacatttcacattgtgtaatatttcctctttttttatcagtaaattacatttacttaaggtttcaagtaccagctttcttgcttctactattgatactccatgtaatttaccagataaagtatctgtctctgcaatgttatgtttaagatcaagagtacctgatttacttattataatctgcgtatttaaattatgtttattccaccaatatacatctaattcatcaccaaatgtacaacacattacaagaccagtacccttatctatttttacttgttcatctgataaaatcggtactttattcccaaatataggtactatagcatactga

SEQ ID NO: 102 - protein, 200 aa, Ehrlichia ruminantium (formerly Cowdria ruminantium)
SIGSSFTSGIIISSLSLFEYHTGNGIPKYLCRDMHQSQFNPSIHISICLRIDCGYQLILRTCSNSSSFISTTLINHWLLNNISIGIPDRSAHFTLCNISSFFISKLHLLKVSSTSFLASTIDTPCNLPDKVSVSAMLCLRSRVPDLLIIICVFKLCLFHQYTSNSSPNVQHITRPVPLSIFTCSSDKIGTLFPNIGTIAY

SEQ ID NO: 103 - DNA, 1321 bp, Ehrlichia ruminantium (formerly Cowdria ruminantium)
  misc_feature (1)..(1321): n = a, c, g, or t
ngatctattggtagttcattcacatctggaataataatctcttcattatctcttttcgaataccataccggaaacgggattccaaaatacctttgtcgcgatatgcaccagtcccagtttaacccatctatccacatttctatttgcttacgcatagactgtggataccaattaatcttacggacctgttctaatagctcatcttttatctctacaaccttaataaaccattggttacttaacaatatttcaataggcattcctgatcgctcagcacatttcacattgtgtaatatttcctctttttttatcagtaaattacatttacttaaggtttcaagtaccagctttcttgcttctactattgatactccatgtaatttaccagataaagtatctgtctctgcaatgttatgtttaagatcaagagtacctgatttacttattataatctgcgtatttaaattatgtttattccaccaatatacatctaattcatcaccaaatgtacaacacattacaagaccagtacccttatctatttttacttgttcatctgataaaatcggtactttattcccaaatataggtactatagcatactgaccttgaagatgctgatatcttatatccaatggattaaaaaataaagcaacacaagctggcattaattctggacgcgtcgttgcaatatttattagctctccagcctccgtagaaaaggctatcgtactcataaacgatgacatttccttttcctcaacctcaactctcgctatcgccgttctatcagcacaatcccaaaatataggctgtaacttcctatatattttacccatattatatagtgctataaatgacatttgagataacttttgaatctcttcacttatagtatgatattccagatcccaatcataactaataccaagagattgaaacaatattttaaattccattctgaattttgcagatacttcattacataatgccttaaattctttacgatcaatatctgtcgcacgtactttttttatcttttcaactaaacgttctgttggcaatccattgtcatcaaatcctattggatatagtacatcttttcctaacatacgttgatatcttgcaataaaatccgtatgacagtagctaaaaacatgtcctatatgtagttgtcctgatattgttggaggaggagtatcaataataaactgtttatcttgtaaattcttccatttatataattttattttatcccaataggtattaagtttttcttctgtatctttgaacttgtatttattactaaaaagagactgca

SEQ ID NO: 104 - DNA, 549 bp, Ehrlichia ruminantium (formerly Cowdria ruminantium)
  misc_feature (1)..(549): Complement to SEQ ID NO100, nucleotides 3508.. >4055 Hypothetical glutamate-cysteine ligase Product = “3hworf3i”
ctaccgatcaaaaacttcaactgcagcagccaatgcagccatcttagcaaccatactccaacagaatattttaccttcttgctctctaaaacttacatctacaaaatcacaacctacagaatttaggttagagaatttatctttaacagtattatacctatataaataacacgaaggagtatcacctatgaaatatactaatggttctgcagtataaccattaaacaactcttcagtcattataccttcttgtattattacacgctcaactattttcctatccttaattcttttcatcttattgcgattttttttatttaacttcaatatgtcatcaccacaatatgctacaataattcccattccatatgttccattatctgcttttacaaacacatacggttgttctgtaatactgtataattgaaatttattacgtatttcttgaatcataacatcaactttatcagcaatgtgttctactccataattacttaaaaaacagatgttatcacaactagaaaataatgtagaaatcaaccaaggatc

SEQ ID NO: 105 - DNA, 4122 bp, Ehrlichia ruminantium (formerly Cowdria ruminantium)
gatcttattatccatgttcccaccgtactgaggatgaggatgaaaaattaaagcaattggagctcctaccgttttattatggtgatactttccttcaatttttcctactgcaccattaaaaaatatctctctcatgaattaacttccttaaaaattgagtaaaccttcacctcatcaataatacaaaaaagtttaacttaataaagtatttttaacaagtgtattattaattacaccttcaaaagcgctatagttttgctttcatttttgtaaaaaaaattaaagttgcaatttaaatacatatttaacaataaatatttttacttatctcatttaagaaaaaacctacttgacacaaggtatattaatgatacattatcaagttgttgataatatcaaaatgatctattcaattaaaaatacttaaaaaggcccataattaacgtgtggatatttatatgttgatcacagctaggctacgttatgctataatgtttatggtaaaattggctcatggtctttgcacgcaacaaaataagttgcaaccagtaagaatgtcatatattgcaagtaatcaatctttatctgaaggatatcttgaacaggtaattgttcaattaaagaaaaaagggcttattaatgctacaaaaggtccaggtggtggttattcactaagtattgctcctcatttaattacacttagtcttatacttgaatcaataggcgaaaatattaaaattacaagatgcgaaaataacagtccaggttgtctatcgaataataatagatgtgtaactcacaaattatgggatgatataggaaattatataaaagattatttaaataatatttcactagaggatatagtaaataataattttaggtcaaacatagcactacataaaaacgaggaaccctatatatatgctgattacaattcaacatctacgatactacctgaagtaaaatatcaattgaataatttatcctatataaaattatataatccatcttcaatacataaactaggtcaaaaaacaaaaagtataatagaagaaacaagaaacatagctattaagcaactaaatgcacaatattatgatgtagtttttacatcctctggtacagaagcaaacaatttagtcatcaatagtacatcagactataaacatttaatttcttctacagaacatctatctattataaaatgtgctactaatgcagaattaatacctgttgattctaatggaataatatgtctaaatgcattgagtagtcttttacataagtttaaagatgacaaaatactagtatcagtaatgacagcaaacaatgaaactggtgctattcaaccaataaaaaaaatcgtagaactatcacataaatttggagcattagtacacacagatgccatacaagcgtgtggaaaaatccatatagatattgaagatttaggagttgatttgttaacaatatcatcacataaacttggcagcattgctggagcaggagtgttattctttaatagcaaaaagataaatataaaacctatgataattggtggacatcaagagaaaggattaagagcaggaactgaaaatgttttagcgatatacttattatctatatcacttagtaatttatataaatctataacaaagatgcttcttgttgaaaaattaagaaataaattggaaaatgaaatattatctttagttcctaacgctcaaatatttagtagaaatgtcgagagactaccaaatactagttgtatttcaatgccaaatgtaaatagtgagattcaagtaataagctttgacataaaaaatattgcagtaggtaacgggtcagcatgttccacaggagtagtagaaccctctcatgtgttatctgcaatgggggtaaatcaggagattgcaaataattcaataaggattagcttaagtcctgacactacagatgagcatataagaactatagtaaactgttggtacgaaatatatacacataatcaagtgcataaatgaaattaggtgtaaaatgatgcaaaaattagaagattactatatcattaacgatatcaaaaataggttataacatggaaaaaataaaagatatacaacgacatattaatctacctatatttcttgattatcaatccacaactaaaacggatagtagagtcttagatgctatgattccatattttcaagagttttctaatcctcattcacgtagtcattgttttggatggaaagctgagtcggctgttgaattagcacgagaaagaattgcatctttaataaatgctgaaagtaaagaagtaatattcacttctggtgctacagaatcaaataatttggcaattaaaggtgtagcaaacttttataaaagtaaaggaaatcacattattacagtacgtacagagcataaatgtgttttagattcatgtcgccatttagaaacagaaggatttgacgttacttacttagatgtacaaaaaaatggtattttagatcttaatttattaaaatcagctataactgataagactatattggtatcggttatgatggtaaacaatgaaattggtgtcattcaaccaatagaggaaataggaaaaatttgtcataatcatggagtattctttcatactgatgcagctcaagcatttggtaaaataccaatagatgtaaacaaaatgaatatagatttgcttagtatatcaggacataaaatatatgctccaatgggaataggggcattatatgtacgtaaacgtcaaccaagaatacgacttactcctataataaatggtggtggacaagaacgtggtatgagatctggaaccatacctactccattagcagtaggtttaggggaagcagcatatatagctcaggaagtaatggaagatgaagccattaggataaaagaattgcgtgatattttatatagtgaaataaaaaaacatttaccctatgtaatactgaatggtgattatgaacaacgtatagcaggaaatttaaatttaagttttccatatgtagaaggagaatctattattatggcgattaacaacttagcagtaagttcaggatctgcttgtacatctgcttcactagaaccatcttatgttttacgtgcgttaaatatagataaggatttagaacactcatctattagatttggaataggtaggtttactacaaaagcagaaattttatatgcagcggatcttattgtaaatagcataaagaggttgcgtgagatgagtcctttatgggaaatggtacaagaaggtataaatcttaatgaaattaagtgggatgtgcattaatttttattgtatttgataattatgaggtatttatatgagttacagtgagtctcttctagaacattataagaatcctaaaaatgttggtactttacctaaagaggattacaatgtaggtactggcttagtgggagctcctagctgtggtgatgtaatgaagttacagattaaagtagatgataatggaaaaattatagatgcaaaatttaaaacttttggatgtggtgctgcaattgcagctagttcactagctactgagttaattaaaggtaaaacagtagatgaggcacatgagttgaaaaatacagtattggcaaaagaattaagtttacctccagtgaaaatacattgttcattacttgcagaagatgctgtaaaagcagctataaatgactatcatatgaaacaagcaaacaaaaaaaatgctactaaagatcctaatgaataatacagcaataatatgcaatcaggtttatctatggataaagaagtgttgataccacataataatcaagagaatacatgtgattcacagataaagtattttataaacagtagtttcacagataagtctcctattatcataacggaaaatgctattaaaaaaattaaggaactcattgataagaaaaaagattctgtcattgggattagaataatggtagcgcaaaaaggatgttttggttttaagtataatatagaatatgcatatgatatcaaaatgttagatgtacaaattcaagtaaaatatcaaaatcaaaattttataattttgattgatc

SEQ ID NO: 106 - DNA, 1569 bp, Ehrlichia ruminantium (formerly Cowdria ruminantium)
  CDS (1)..(1569): Corresponds to SEQ ID NO105, nucleotides 459..2027 Hypothetical iron-sulfur co-factor synthesis Product = “11hworf1”
atgttgatcacagctaggctacgttatgctataatgtttatggtaaaattggctcatggtctttgcacgcaacaaaataagttgcaaccagtaagaatgtcatatattgcaagtaatcaatctttatctgaaggatatcttgaacaggtaattgttcaattaaagaaaaaagggcttattaatgctacaaaaggtccaggtggtggttattcactaagtattgctcctcatttaattacacttagtcttatacttgaatcaataggcgaaaatattaaaattacaagatgcgaaaataacagtccaggttgtctatcgaataataatagatgtgtaactcacaaattatgggatgatataggaaattatataaaagattatttaaataatatttcactagaggatatagtaaataataattttaggtcaaacatagcactacataaaaacgaggaaccctatatatatgctgattacaattcaacatctacgatactacctgaagtaaaatatcaattgaataatttatcctatataaaattatataatccatcttcaatacataaactaggtcaaaaaacaaaaagtataatagaagaaacaagaaacatagctattaagcaactaaatgcacaatattatgatgtagtttttacatcctctggtacagaagcaaacaatttagtcatcaatagtacatcagactataaacatttaatttcttctacagaacatctatctattataaaatgtgctactaatgcagaattaatacctgttgattctaatggaataatatgtctaaatgcattgagtagtcttttacataagtttaaagatgacaaaatactagtatcagtaatgacagcaaacaatgaaactggtgctattcaaccaataaaaaaaatcgtagaactatcacataaatttggagcattagtacacacagatgccatacaagcgtgtggaaaaatccatatagatattgaagatttaggagttgatttgttaacaatatcatcacataaacttggcagcattgctggagcaggagtgttattctttaatagcaaaaagataaatataaaacctatgataattggtggacatcaagagaaaggattaagagcaggaactgaaaatgttttagcgatatacttattatctatatcacttagtaatttatataaatctataacaaagatgcttcttgttgaaaaattaagaaataaattggaaaatgaaatattatctttagttcctaacgctcaaatatttagtagaaatgtcgagagactaccaaatactagttgtatttcaatgccaaatgtaaatagtgagattcaagtaataagctttgacataaaaaatattgcagtaggtaacgggtcagcatgttccacaggagtagtagaaccctctcatgtgttatctgcaatgggggtaaatcaggagattgcaaataattcaataaggattagcttaagtcctgacactacagatgagcatataagaactatagtaaactgttggtacgaaatatatacacataatcaagtgcataaatga

SEQ ID NO: 107 - protein, 522 aa, Ehrlichia ruminantium (formerly Cowdria ruminantium)
MLITARLRYAIMFMVKLAHGLCTQQNKLQPVRMSYIASNQSLSEGYLEQVIVQLKKKGLINATKGPGGGYSLSIAPHLITLSLILESIGENIKITRCENNSPGCLSNNNRCVTHKLWDDIGNYIKDYLNNISLEDIVNNNFRSNIALHKNEEPYIYADYNSTSTILPEVKYQLNNLSYIKLYNPSSIHKLGQKTKSIIEETRNIAIKQLNAQYYDVVFTSSGTEANNLVINSTSDYKHLISSTEHLSIIKCATNAELIPVDSNGIICLNALSSLLHKFKDDKILVSVMTANNETGAIQPIKKIVELSHKFGALVHTDAIQACGKIHIDIEDLGVDLLTISSHKLGSIAGAGVLFFNSKKINIKPMIIGGHQEKGLRAGTENVLAIYLLSISLSNLYKSITKMLLVEKLRNKLENEILSLVPNAQIFSRNVERLPNTSCISMPNVNSEIQVISFDIKNIAVGNGSACSTGVVEPSHVLSAMGVNQEIANNSIRISLSPDTTDEHIRTIVNCWYEIYTHNQVHK

SEQ ID NO: 108 - DNA, 1242 bp, Ehrlichia ruminantium (formerly Cowdria ruminantium)
  CDS (1)..(1242): Corresponds to SEQ ID NO105, nucleotides 2099..3340 Hypothetical iron-sulfur co-factor synthesis Product = “11hworf2”
atggaaaaaataaaagatatacaacgacatattaatctacctatatttcttgattatcaatccacaactaaaacggatagtagagtcttagatgctatgattccatattttcaagagttttctaatcctcattcacgtagtcattgttttggatggaaagctgagtcggctgttgaattagcacgagaaagaattgcatctttaataaatgctgaaagtaaagaagtaatattcacttctggtgctacagaatcaaataatttggcaattaaaggtgtagcaaacttttataaaagtaaaggaaatcacattattacagtacgtacagagcataaatgtgttttagattcatgtcgccatttagaaacagaaggatttgacgttacttacttagatgtacaaaaaaatggtattttagatcttaatttattaaaatcagctataactgataagactatattggtatcggttatgatggtaaacaatgaaattggtgtcattcaaccaatagaggaaataggaaaaatttgtcataatcatggagtattctttcatactgatgcagctcaagcatttggtaaaataccaatagatgtaaacaaaatgaatatagatttgcttagtatatcaggacataaaatatatgctccaatgggaataggggcattatatgtacgtaaacgtcaaccaagaatacgacttactcctataataaatggtggtggacaagaacgtggtatgagatctggaaccatacctactccattagcagtaggtttaggggaagcagcatatatagctcaggaagtaatggaagatgaagccattaggataaaagaattgcgtgatattttatatagtgaaataaaaaaacatttaccctatgtaatactgaatggtgattatgaacaacgtatagcaggaaatttaaatttaagttttccatatgtagaaggagaatctattattatggcgattaacaacttagcagtaagttcaggatctgcttgtacatctgcttcactagaaccatcttatgttttacgtgcgttaaatatagataaggatttagaacactcatctattagatttggaataggtaggtttactacaaaagcagaaattttatatgcagcggatcttattgtaaatagcataaagaggttgcgtgagatgagtcctttatgggaaatggtacaagaaggtataaatcttaatgaaattaagtgggatgtgcattaa

SEQ ID NO: 109 - protein, 413 aa, Ehrlichia ruminantium (formerly Cowdria ruminantium)
MEKIKDIQRHINLPIFLDYQSTTKTDSRVLDAMIPYFQEFSNPHSRSHCFGWKAESAVELARERIASLINAESKEVIFTSGATESNNLAIKGVANFYKSKGNHIITVRTEHKCVLDSCRHLETEGFDVTYLDVQKNGILDLNLLKSAITDKTILVSVMMVNNEIGVIQPIEEIGKICHNHGVFFHTDAAQAFGKIPIDVNKMNIDLLSISGHKIYAPMGIGALYVRKRQPRIRLTPIINGGGQERGMRSGTIPTPLAVGLGEAAYIAQEVMEDEAIRIKELRDILYSEIKKHLPYVILNGDYEQRIAGNLNLSFPYVEGESIIMAINNLAVSSGSACTSASLEPSYVLRALNIDKDLEHSSIRFGIGRFTTKAEILYAADLIVNSIKRLREMSPLWEMVQEGINLNEIKWDVH

SEQ ID NO: 110 - DNA, 414 bp, Ehrlichia ruminantium (formerly Cowdria ruminantium)
  CDS (1)..(414): Corresponds to SEQ ID NO105, nucleotides 3375- 3788 NIFU-like protein Product  =“11hworf3”
atgagttacagtgagtctcttctagaacattataagaatcctaaaaatgttggtactttacctaaagaggattacaatgtaggtactggcttagtgggagctcctagctgtggtgatgtaatgaagttacagattaaagtagatgataatggaaaaattatagatgcaaaatttaaaacttttggatgtggtgctgcaattgcagctagttcactagctactgagttaattaaaggtaaaacagtagatgaggcacatgagttgaaaaatacagtattggcaaaagaattaagtttacctccagtgaaaatacattgttcattacttgcagaagatgctgtaaaagcagctataaatgactatcatatgaaacaagcaaacaaaaaaaatgctactaaagatcctaatgaataa

SEQ ID NO: 111 - protein, 137 aa, Ehrlichia ruminantium (formerly Cowdria ruminantium)
MSYSESLLEHYKNPKNVGTLPKEDYNVGTGLVGAPSCGDVMKLQIKVDDNGKIIDAKFKTFGCGAAIAASSLATELIKGKTVDEAHELKNTVLAKELSLPPVKIHCSLLAEDAVKAAINDYHMKQANKKNATKDPNE

SEQ ID NO: 112 - protein, 15 aa, Unknown
  Hypothetical sequence
PTLVTLSVCHFGIEL

SEQ ID NO: 113 - protein, 13 aa, Unknown
  Hypothetical sequence
LVTLSVCHFGIEL

SEQ ID NO: 114 - DNA, 18 bp, Unknown
  Primer
cggggtaccgaattcctc

SEQ ID NO: 115 - DNA, 18 bp, Unknown
  Primer
gcatgctcctctagactc

SEQ ID NO: 116 - protein, 9 aa, Ehrlichia ruminantium (formerly Cowdria ruminantium)
VTSSPEGSV

SEQ ID NO: 117 - protein, 9 aa, Unknown
  Synthetic peptide
VTSSPEGSV